(19) United States
(12) Patent Application Publication
(10) Pub. No.: US 2024/0140991 A1
(43) Pub. Date: May 2, 2024

(54) ANTIBIOTIC NATURAL PRODUCT ANALOGUES

(71) Applicants: THE QUEEN'S UNIVERSITY OF BELFAST, Belfast, Antrim (GB); UNIVERSITEIT LEIDEN, EZ Leiden (NL)

(72) Inventors: Stephen COCHRANE, Belfast (GB); Ross BALLANTINE, Belfast (GB); Nathaniel MARTIN, Leiden (NL); Karol AL AYED, Leiden (NL); Michael HOEKSTRA, Leiden (NL); Sofia Denise Zamarbide LOSADA, Leiden (NL)

(21) Appl. No.: 18/263,335

(22) PCT Filed: Nov. 30, 2021
(86) PCT No.: PCT/GB2021/053123

§ 371 Date: Jul. 27, 2023

(30) Foreign Application Priority Data
Jan. 28, 2021 (GB) 2101165.5

(51) Int. Cl. C07K 7/64; A61P 31/04; C07K 7/08

(52) U.S. Cl. CPC C07K 7/64; A61P 31/04; C07K 7/08; A61K 38/00

(57) ABSTRACT

A compound of formula (I) or formula (II) including tautomeric or stereochemically isomeric forms thereof, wherein: R¹ represents C1-20 alkyl, C2-20 alkenyl, C6-20 aryl, or C4-20 heterocyclyl, each optionally substituted with one or more Y groups, R^{2a} to R^{2l} each independently represents H, C1-10 alkyl, C2-10 alkenyl, C6-12 aryl, or C4-12 heterocyclyl, each optionally substituted with one or more Y groups, R³ represents H, C1-10 alkyl, C2-10 alkenyl, C6-12 aryl, or C4-12 heterocyclyl, each optionally substituted with one or more Y groups, X represents NH, S or O, each Y independently represents cyano, halogen, N₃, —C(O)R^{Z}, —C(0)0R^{z}, —0C(0)R^{z}, —C(0)NH^{Z}, —NHC(O)R^{z}, —NHC(0)NHR^{Z}, —NHC(NH)NHR^{Z}, —NCH(0)0R^{z}, -0C(0)NHR^{z}, -0S(0)₂R^{Z}, —S(0)₂NHR^{Z}, —NHS(0)₂R^{Z}, —SR^{Z}, —NR^{Z}Z or —OR^{z} and each R^{z} independently represents H, C1-10 alkyl, C2-10 alkenyl, C6-10 aryl, or C4-10 heterocyclyl; or an N-oxide thereof or a pharmaceutically acceptable salt thereof or a pharmaceutically acceptable solvate thereof; and wherein formula (I) excludes laterocidine, wherein formula (I) excludes relacidine A, and wherein formula (I) excludes relacidine B. A pharmaceutical composition comprising the compound. The compounds and pharmaceutical compositions are useful as antibiotics.

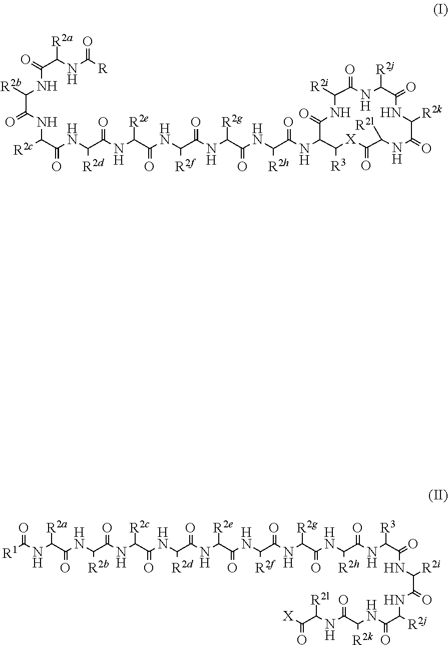

### CROSS-REFERENCE TO RELATED APPLICATION

The present application claims priority to PCT/GB2021/053123, filed Nov. 30, 2021, titled “Antibiotic Natural Product Analogues, which claims priority to GB 2101165.5, filed Jan. 28, 2021, both of which are incorporate by reference herein in their entireties.

### REFERENCE TO AN ELECTRONIC SEQUENCE LISTING

The Sequence Listing associated with this application is provided in text format in lieu of a paper copy, and is hereby incorporated by reference into the specification.

The name of the text file containing the Sequence Listing is Sequence Listing_031221.txt. The text file is 7 KB, was created on Jul. 2, 2023, and is being submitted electronically via Patent Center.

### FIELD OF THE INVENTION

The present invention relates to compounds that are synthetic analogues of natural products. The present invention also relates to pharmaceutical compositions comprising such compounds and the use of such compounds or pharmaceutical compositions as a medicament, in particular as an antibiotic.

### BACKGROUND OF THE INVENTION

Combatting antibiotic resistance is one of the most significant challenges facing our generation. A 2014 report predicted that by 2050 antibiotic resistance will cause 300 million premature deaths and cost the global economy over $100 trillion. The World Health Organization lists as critical priority pathogens the Gram-negative bacteria carbapenem-resistant Enterobacteriaceae (e.g. Escherichia coli), Pseudomonas aeruginosa, Acinetobacter baumannii, and Klebsiella pneumoniae. Such Gram-negative bacteria can be significantly more difficult to target than Gram-positive bacteria because the outer membrane of Gram-negative bacteria blocks many antibiotics.

As such, new antibiotics, in particular those that target Gram-negative bacteria, are desperately needed.

### SUMMARY OF THE INVENTION

The present invention provides, according to a first aspect, a compound of formula (I) or formula (II):

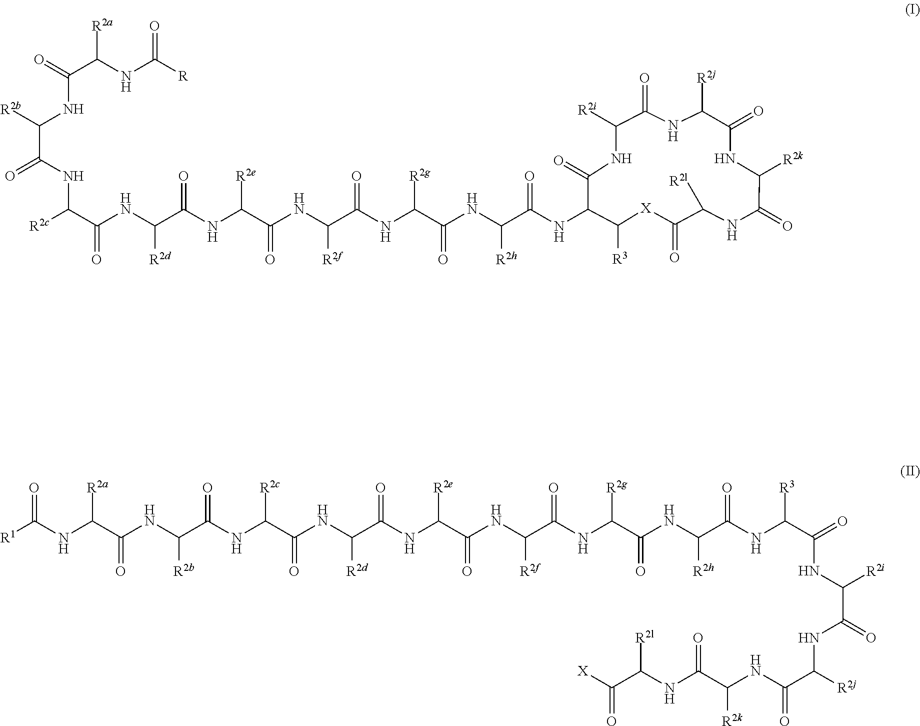

including tautomeric or stereochemically isometric forms thereof, wherein:
R¹ represents C1-20 alkyl, C2-20 alkenyl, C6-20 aryl, or C4-20 heterocyclyl, each optionally substituted with one of more Y groups,
R^{2a} to R^{2l} each independently represents H, C1-10 alkyl, C2-10 alkenyl, C6-12 aryl, or C4-12 heterocyclyl, each optionally substituted with one or more Y groups,
R³ represents H C1-10 alkyl, C2-10 alkenyl, C6-12 aryl, C4-12 heterocyclyl, each optionally substituted with one or more Y groups,
X represents NH, S or O,
each Y independently represents cyano, halogen, N₃, —C(O)R^{Z}, —C(O)OR^{Z}, —OC(O)R^{Z}, —C(O)NHR^{Z}, —NHC(O)R^{Z}, —NHC(O)NHR^{Z}, —NHC(NH)NHR^{Z}, —NHC(O)OR^{Z}, —OC(O)NHR^{Z}, —OS(O)₂R^{Z}, —S(O)₂NHR^{Z}, —NHS(O)₂R^{Z}, —SR^{Z}, —NR^{Z}₂ or —OR^{Z}, and
each R^{Z} independelty represents H, C1-10 alkyl, C2-10 alkenyl, C6-10 aryl, or C4-10 heterocyclyl;
or an N-oxide thereof or a pharmaceutically acceptable salt thereof or a pharmaceutically acceptable solvate thereof;
   wherein formula (I) excludes laterocidine:

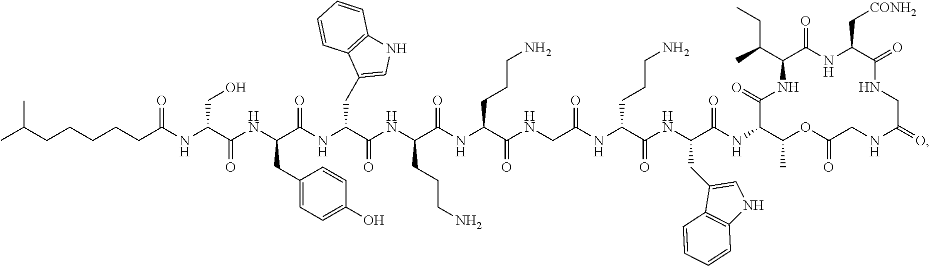 [SMILES: CC[C@H](C)[C@@H]1NC(=O)[C@@H](NC(=O)[C@H](Cc2c[nH]c3ccccc23)NC(=O)[C@@H](CCCN)NC(=O)CNC(=O)[C@H](CCCN)NC(=O)[C@@H](CCCN)NC(=O)[C@@H](Cc2c[nH]c3ccccc23)NC(=O)[C@@H](Cc2ccc(O)cc2)NC(=O)[C@@H](CO)NC(=O)CCCCCC(C)C)[C@@H](C)OC(=O)CNC(=O)CNC(=O)[C@H](CC(N)=O)NC1=O]

wherein formula (I) excludes relacidine A:

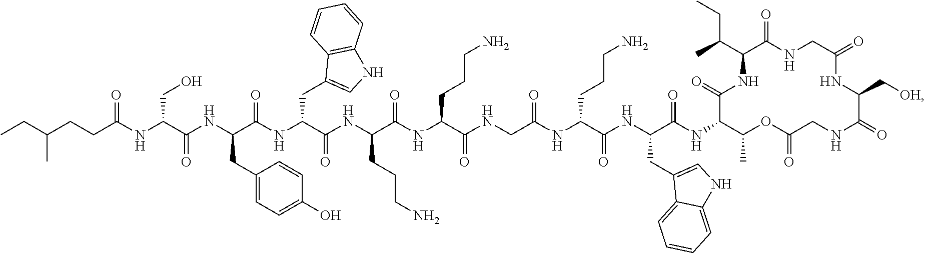 [SMILES: CCC(C)CCC(=O)N[C@H](CO)C(=O)N[C@H](Cc1ccc(O)cc1)C(=O)N[C@H](Cc1c[nH]c2ccccc12)C(=O)N[C@H](CCCN)C(=O)N[C@@H](CCCN)C(=O)NCC(=O)N[C@H](CCCN)C(=O)N[C@@H](Cc1c[nH]c2ccccc12)C(=O)N[C@@H]1C(=O)N[C@@H]([C@@H](C)CC)C(=O)NCC(=O)N[C@@H](CO)C(=O)NCC(=O)O[C@@H]1C]

and wherein formula (I) excludes relacidine B:

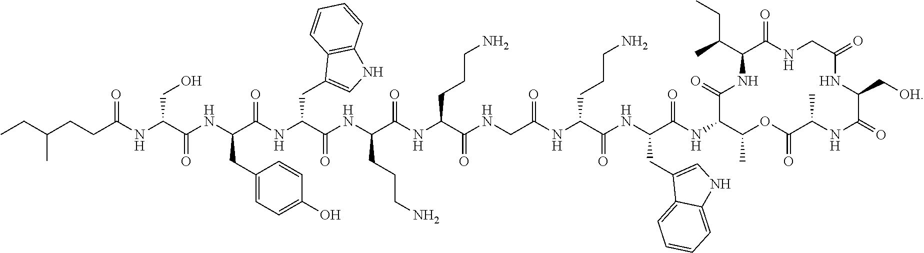 [SMILES: CCC(C)CCC(=O)N[C@H](CO)C(=O)N[C@H](Cc1ccc(O)cc1)C(=O)N[C@H](Cc1c[nH]c2ccccc12)C(=O)N[C@H](CCCN)C(=O)N[C@@H](CCCN)C(=O)NCC(=O)N[C@H](CCCN)C(=O)N[C@@H](Cc1c[nH]c2ccccc12)C(=O)N[C@@H]1C(=O)N[C@@H]([C@@H](C)CC)C(=O)NCC(=O)N[C@@H](CO)C(=O)N[C@@H](C)C(=O)O[C@@H]1C]

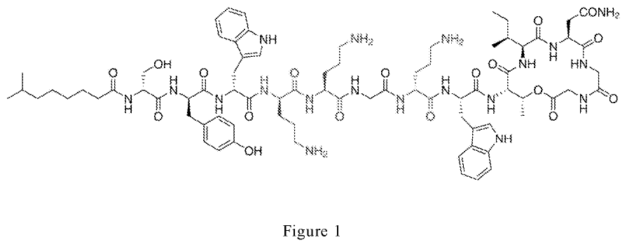

The isolation of the natural product laterocidine and initial investigations into its biological activity were reported by Li et al. (Nat. Commun. 2018, 9, 3273). FIG. 1 shows the chemical structure of laterocidine. It was found that laterocidine exhibits bactericidal activity against several multi-drug resistant Gram-negative bacteria, including Pseudomonas aeruginosa and colistin-resistant Escherichia coli. Laterocidine was found to rival or improve on the antibacterial efficacy of existing antibiotics such as polymyxin B and colistin against certain strains of bacteria. Li et al. also reported that laterocidine, and its relative brevicidine, showed little propensity to induce resistance and had low toxicity towards mammalian cells.

However, laterocidine was only isolated in low amounts (sub-milligram-per-litre yields) from bacterial fermentation. Such isolation methods are not suitable for large-scale production, or for in vivo efficacy studies.

Li et al. (Environ Microbiol. 2020 December; 22(12): 5125-5136) describes the characterisation of the natural products relacidine A and relacidine B. The most likely stereochemical isomer of these natural products is excluded from the first aspect.

It is therefore desirable to provide a synthetic route to analogues of laterocidine and relacidine, such that these compounds can be produced on a large-scale.

It is also desirable to provide new analogues of laterocidine and relacidine to optimise desirable characteristics such as efficacy and/or stability.

The present application enables the synthesis of analogues of laterocidine and relacidine, therefore meeting these needs. Specifically, the ability to synthesise derivatives of naturally occurring laterocidine, as detailed herein, enables the large-scale production of such compounds, and therefore makes the use of such compounds for the treatment of patients viable.

Compounds of formula (I) retain the macrocyclic motif of laterocidine, whereas compounds of formula (II) are “linear” analogues where this macrocyclic motif has been opened. These compounds are synthetic analogues of laterocidine.

The synthetic techniques used to prepare analogues of laterocidine, as described herein, enable the adaption of the structure to provide alternative related compounds and, for example, optimise activity.

Yet, as described in the Examples section, the synthesis of the compounds of the present invention was not facile. Initial studies were hampered by failure. Surprising challenges faced the inventors in the synthesis of these compounds.

However, the inventors have overcome these problems and have, therefore, enabled the synthesis of the compounds of the present invention.

This provides new and useful compounds for the benefit of humankind.

The present inventors have also shown that compounds according to the invention target bacteria, especially Gram-negative bacteria such as E. coli. Klebsiella pneumoniae, Acinetobacter baumannii, and P. aeruginosa. Therefore, compounds according to the invention may provide particularly useful treatments for an infection caused by one or more of these bacteria. Specifically, these compounds may be used in the treatment and/or prevention of bacterial infections. As such, these compounds may be used as antibiotics.

Importantly, compounds of the present invention retain activity against multi-drug resistant strains of bacteria, such as mcr-positive polymyxin resistant strains of bacteria.

The activity of the compounds of the present invention against Gram-negative bacteria is particularly notable owing to the difficulty to target such bacteria with existing antibiotics.

It is also particularly notable that the inventors have shown that compounds of the present invention are well-tolerated and effective at treating antibiotic infection in an in vivo murine thigh-infection model.

The compounds of the present invention have also been shown have selectivity against Gram-negative bacteria such as E. coli, K. pneumoniae, A. baumannii, and P. aeruginosa as opposed to Gram-positive bacteria. This may allow for a targeted treatment against Gram-negative bacteria, i.e. which does not simultaneously target Gram-positive bacteria that may be associated with a normal or healthy condition of the human body.

Therefore, the present invention addresses the need for new antibiotics by providing novel non-natural peptides for the treatment or prevention of bacterial infection, in particular those caused by a range of Gram-negative bacteria such as E. coli, K. pneumoniae, A. baumannii, and P. aeruginosa.

Benefits of the compounds of the present invention include one or more of: good efficacy against a range of bacteria, especially Gram-negative bacteria, including strains of E. coli, K. pneumoniae. A. baumannii, and/or P. aeruginosa; and/or selective activity against Gram-negative bacteria as compared to Gram-positive bacteria that may be associated with good health.

Furthermore, it has been determined that the stability of these compounds can be improved where X is NH, as opposed to being O in the natural product.

The antibiotic activity of analogues of the related natural product brevicidine (also reported by Li et al., Nat. Commun. 2018, 9, 3273) generally decreases when this substitution of X being O to X being NH is made. However, surprisingly, the antibiotic activity of laterocidine analogues is substantially retained when this alteration is made.

Li et al. (Nat. Commun. 2018, 9, 3273) have previously suggested that the macrocycle unit is necessary for strong antibacterial activity of these peptides.

However, the present inventors have surprisingly determined that the macrocycle is not strictly necessary to achieve the antibacterial properties. In particular, the present inventors have shown that linear analogues of laterocidine and other related compounds retain antibacterial activity of the structure that includes the macrocyclic motif.

Compounds of the present invention have been found to be non-hemolytic.

Compounds of the present invention have been shown to have low toxicity, or be non-toxic, to HepG2 cells.

In particular, compounds such as laterocidamide display a particularly favourable balance of antibacterial activity, stability, low cell toxicity, and synthetic accessibility.

Replacement of the naturally occurring ester motif with an amide motif has been found to be particularly well tolerated by the larger 5-amino acid macrocycles found in laterocidine and relacdine.

A combination of these beneficial characteristics can make compounds of the present invention highly advantageous candidates for antibiotic use.

According to a second aspect, the invention provides a pharmaceutical composition comprising the compound of the first aspect and a pharmaceutically acceptable carrier or diluent.

According to a third aspect, the invention provides a compound according to the first aspect or a pharmaceutical composition according to the second aspect for use in therapy.

According to a fourth aspect, the invention provides a compound according to the first aspect or a pharmaceutical composition according to the second aspect for use as an antibiotic. In particular, the use may be as an antibiotic against Gram-negative bacteria such as E. coli, K. pneumoniae, A. baumannii, and/or P. aeruginosa. The use may be in the treatment or prevention of bacterial infections, in particular those caused by Gram-negative bacteria such as E. coli, K. pneumoniae, A. baumannii, and/or P. aeruginosa, or strains thereof.

According to a fifth aspect, the invention provides a method of making a pharmaceutical composition according to the second aspect, the method comprising the step of mixing a compound according to the first aspect with a pharmaceutically acceptable carrier or diluent.

According to a sixth aspect, the invention provides a method of treating individuals suffering from bacterial infection, the method comprising administering an effective amount of a compound according to the first aspect or a pharmaceutical composition according to the second aspect.

### DETAILED DESCRIPTION

The prefix “Cx-y” (where x and y are integers) as used herein refers to the number of carbon atoms in a given group. For example, a C1-6 alkyl group contains from 1 to 6 carbon atoms, and a C3-6 alkyl group contains from 3 to 6 carbon atoms. The term “C(O)” refers to a carbonyl group, i.e. a carbon atom double bonded to an oxygen atom.

The term “alkyl” refers to linear and branched saturated hydrocarbon groups. Examples of such groups include methyl, ethyl, n-propyl, isopropyl, n-butyl, isobutyl, sec-butyl, tert-butyl, n-pentyl, isopentyl, neopentyl or hexyl and the like. The term “alkyl” can include “cycloalkyl” groups, i.e. cyclic hydrocarbon groups. Examples of such groups include cyclopropyl, cyclobutyl, cyclopentyl, or cyclohexyl and the like.

The term “alkenyl” as used herein refers to a linear or branched hydrocarbon group containing one or more carbon-carbon double bond. Examples of such groups include vinyl, allyl, prenyl, isoprenyl and the like.

The term “aryl” as used herein refers to carbocyclic aromatic groups including phenyl, naphthyl, indenyl, and tetrahydronaphthyl groups. An aryl group may also include, within the number of carbon atoms defined, alkyl or alkenyl groups as defined above. For example, toluene could be considered to be a C7 aryl group.

The term “heterocyclyl” as used herein shall, unless the context indicates otherwise, include both aromatic (i.e. heteroaryl) and non-aromatic ring systems. Thus, for example, the term “heterocyclyl group” includes within its scope aromatic, non-aromatic, unsaturated, partially saturated and fully saturated heterocyclyl ring systems.

In general, unless the context indicates otherwise, such groups may be monocyclic or bicyclic and may contain, for example, 4 to 10 ring members, more usually 5 to 10 ring members. A heterocyclyl group may also include, within the number of carbon atoms defined, alkyl or alkenyl groups as defined above. For example, methyl pyridine could be considered to be a C6 heteroaryl group.

Examples of monocyclic groups are groups containing 4, 5, 6, 7 and 8 ring members, more usually 4 to 7, and preferably 5, 6 or 7 ring members, more preferably 5 or 6 ring members. Examples of bicyclic groups are those containing 8, 9 and 10 ring members. Typical examples of saturated heterocyclic groups include aziridines, oxiranes, pyrrolidines, piperidines, piperazines and decahydroisoquinolines.

The heterocyclyl groups can be heteroaryl groups. Such groups may have from 5 to 10 ring members. The term “heteroaryl” is used herein to denote a heterocyclyl group having aromatic character. The term “heteroaryl” embraces polycyclic (e.g. bicyclic) ring systems wherein one or more rings are non-aromatic, provided that at least one ring is aromatic. In such polycyclic systems, the group may be attached by the aromatic ring, or by a non-aromatic ring.

The heteroaryl group can be, for example, a five membered or six membered monocyclic ring or a bicyclic structure formed from fused five and six membered rings or two fused six membered rings. Each ring may contain up to five heteroatoms typically selected from nitrogen, sulphur and oxygen. Typically the heteroaryl ring will contain up to 4 heteroatoms, more typically up to 3 heteroatoms, more usually up to 2, for example a single heteroatom. In one embodiment, the heteroaryl ring contains at least one ring nitrogen atom. Examples of heteroaryl groups include but are not limited to indole, pyrrole, furan, thiophene, imidazole, furazan, oxazole, oxadiazole, oxatriazole, isoxazole, thiazole, thiadiazole, isothiazole, pyrazole, triazole and tetrazole, pyridine, pyrazine, pyridazine, pyrimidine and triazine groups.

In the compound of formula (I) and (II):
R¹ represents C1-20 alkyl, C2-20 alkenyl, C6-20 aryl, or C4-20 heterocyclyl, each optionally substituted with one or more Y groups,
R^{2a} to R^{2l} each independently represents H, C1-10 alkyl, C2-10 alkenyl, C6-12 aryl, or C4-12 heterocyclyl, each optionally substituted with one or more Y groups,
R³ represents H, C1-10 alkyl, C2-10 alkenyl, C6-12 aryl, or C4-12 heterocyclyl, each optionally substituted with one or more Y groups,
X represents NH, S or O,
each Y independently represents cyano, halogen, N₃, —C(O)R^{Z}, —C(O)OR^{Z}, —OC(O)R^{Z}, —C(O)NHR^{Z}, —NHC(O)R^{Z}, —NHC(O)NHR^{Z}, —NHC(NH)NHR^{Z}, —NHC(O)OR^{Z}, —OC(O)NHR^{Z}, —OS(O)₂R^{Z}, —S(O)₂NHR^{z}, —NHS(O)₂R^{Z}, —SR^{Z}, —NR^{Z}₂ or —OR^{z}, and
each R^{Z} independently represents H, C1-10 alkyl, C2-10 alkenyl, C6-10 aryl, or C4-10 heterocyclyl.

Therefore, the groups of R¹, R² and/or R³ may be substituted by one or more Y groups. For example, these groups may each or all be substituted by two or more, or three or more Y groups. The groups of R¹, R² and/or R³ may be substituted by four or fewer Y groups, such as three or fewer Y groups, preferably only one or two Y groups, more preferably only one Y group.

R¹ may represent C4-20 alkyl, C4-20 alkenyl, C6-20 aryl, or C4-20 heterocyclyl, each optionally substituted with one or more Y groups. R¹ preferably represents C6-20 alkyl, C6-20 alkenyl, C6-20 aryl, or C6-20 heterocyclyl, each optionally substituted with one or more Y groups. For example, R¹ may represent C8-20 alkyl, C8-20 alkenyl, C8-20 aryl, or C8-20 heterocyclyl, each optionally substituted with one or more Y groups. R¹ may represent C1-16 alkyl, C2-16 alkenyl, C6-16 aryl, or C4-16 heterocyclyl, each optionally substituted with one or more Y groups. For example, R¹ may represent C1-14 alkyl, C2-14 alkenyl, C6-14 aryl, or C4-14 heterocyclyl, each optionally substituted with one or more Y groups. Preferably R¹ represents C1-12 alkyl, C2-12 alkenyl, C6-12 aryl, or C4-12 heterocyclyl, each optionally substituted with one or more Y groups.

R¹ may represent C4-16 alkyl, C4-16 alkenyl, C6-16 aryl, or C4-16 heterocyclyl, each optionally substituted with one or more Y groups. Preferably R¹ represents C6-12 alkyl, C6-12 alkenyl, C6-12 aryl, or C6-12 heterocyclyl, each optionally substituted with one or more Y groups. For example, R¹ may represent C8-12 alkyl, C8-12 alkenyl, C8-12 aryl, or C8-12 heterocyclyl, each optionally substituted with one or more Y groups.

R¹ may represent C1-20 alkyl, C2-20 alkenyl, or C6-20 aryl, each optionally substituted with one or more Y groups. For example, R¹ may represent C6-12 alkyl, C6-12 alkenyl, or C6-12 aryl, each optionally substituted with one or more Y groups. Preferably R¹ represents C1-20 alkyl, or C2-20 alkenyl, each optionally substituted with one or more Y groups. For example, R¹ may represent C6-12 alkyl, or C6-12 alkenyl, each optionally substituted with one or more Y groups. More preferably, R¹ represents C1-20 alkyl, such as C6-12 alkyl, optionally substituted with one or more Y groups.

R¹ is preferably substituted with from 0 to 3 Y groups, for example from 0 to 2 Y groups. More preferably R¹ is substituted with 0 or 1 Y group. Most preferably R¹ is unsubstituted, for example R¹ may represent unsubstituted C1-20 alkyl, unsubstituted C2-20 alkenyl, or unsubstituted C6-20 aryl, for example unsubstituted C6-12 alkyl, unsubstituted C6-12 alkenyl, or unsubstituted C6-12 aryl. For example, R¹ may represent a 6-methylheptyl group:

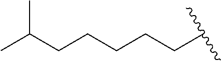 [SMILES: *CCCCCC(C)C]

Any Y groups of R¹ are preferably each independently selected from the list consisting of cyano, halogen, —C(O)OR^{Z}, —C(O)NHR^{Z}, —S(O)₂NHR^{z}, —NHS(O)₂R^{Z}, —NR^{Z}₂ or —OR^{z}.

More preferably, any Y groups of R¹ are each independently selected from the list consisting of —NR^{Z}₂ and —OR^{z}. Most preferably any Y groups of R¹ are —OR^{z}. In each such case, preferably, R^{Z} is H. Y may be —SR^{Z}.

It will be noted that compounds of the present disclosure include a plurality of R² groups, R^{2a} to R^{2l}. Each of these R² groups may represent different groups, or some R² groups may represent the same functional group(s), or all R² groups may represent the same functional group. Preferably some R² groups have common functional groups. R^{2a} to R^{2l} may each independently represent H, C1-6 alkyl, C2-6 alkenyl, C6-12 aryl, C4-12 heterocyclyl, each optionally substituted with one or more Y groups.

R^{2a} to R^{2l} is each preferably substituted with from 0 to 3 Y groups, for example from 0 to 2 Y groups. More preferably each R^{2a} to R^{2l} is each independently substituted with 0 or 1 Y group. Any Y groups may be each independently selected from the list consisting of cyano, halogen (e.g. F, C1 or Br), N₃, —C(O)OR^{Z}, —C(O)NHR^{Z}, —S(O)₂NHR^{z}, —NHS(O)₂R^{Z}, —NR^{Z}₂ or —OR^{z}. Y may be —NHC(NH)NHR^{Z} or —SR^{Z}. Preferably any Y groups are each independently selected from the list consisting of N₃, —C(O)OR^{Z}, OC(O)R^{Z}, —C(O)NHR^{Z}, —NHC(O)R^{Z}, —NHC(O)NHR^{Z}, —NHC(O)OR^{Z}, OC(O)NHR^{Z}, —NR^{Z}₂ or —OR^{z}. More preferably, any Y groups are each independently selected from the list consisting of N₃, —C(O)NHR^{Z}, —NHC(O)OR^{Z}, —NR^{Z}₂ or —OR^{z}. Also more preferable is for Y to be —NHC(NH)NHR^{Z} or —SR^{Z}. In each such case, preferably, R^{Z} is H. For example, preferably any Y groups are each independently selected from the list consisting of N₃, —C(O)NH₂, —NHC(O)O(C1-6 alkyl or C2-6 alkenyl, e.g. allyl), —C(O)OH, —NH₂ and —OH. Y may be —NHC(NH)NH₂ or —SH.

From one to six of the groups R^{2a} to R^{2l} may represent C1-10 alkyl, C2-10 alkenyl, C6-12 aryl, or C4-12 heterocyclyl, each substituted with from one to three Y groups represented by NR^{Z}₂, such as NH₂. For example, from one to six of the groups R^{2a} to R^{2l} may represent C1-6 alkyl, C2-6 alkenyl, C6-8 aryl, or C6-8 heterocyclyl, each substituted with from one to three Y groups represented by NR^{Z}₂, such as NH₂. Preferably from two to four, most preferably three, of the groups R²² to R^{2l} represent C1-10 alkyl, C2-10 alkenyl, C6-12 aryl, or C4-12 heterocyclyl, each substituted with from one to three NR^{Z}₂, such as NH₂. For example, preferably from two to four, most preferably three, of the groups R²² to R^{2l} represent C1-6 alkyl, C2-6 alkenyl, C6-8 aryl, or C4-8 heterocyclyl, each substituted with from one to three NR^{Z}₂, such as NH₂. Preferably the one to six, such as two to four, R² groups that represent NR^{Z}₂, such as NH₂, are those from R^{2b} to R²ⁱ, such as from R^{2c} to R^{2h}, in the structures of the compounds of the present disclosure. One, two or, preferably, all three of R^{2d}, R^{2e} and R^{2g} may represent such functional groups.

R^{2a} may represent C1-6 alkyl or C2-6 alkenyl, each optionally substituted with one or more Y groups. R^{2a} preferably represents C1-4 alkyl or C2-4 alkenyl, more preferably only C1-4 alkyl, each optionally substituted with one or more Y groups. Most preferably R^{2a} represents C1 or C2 alkyl, each optionally substituted with one or more Y groups. It will be appreciated that in one embodiment R^{2a} is not substituted. R^{2a} may be substituted with a Y group represented by —C(O)OR^{Z}, —C(O)NHR^{Z}, —S(O)₂NHR^{Z}, —NR^{Z}₂ or —OR^{Z}. R^{2a} is preferably substituted with a Y group represented by —C(O)OH, —C(O)NH₂, —S(O)₂NH₂, —NH₂ or —OH. R²² is preferably substituted with a Y group represented by —NH₂ or —OH, most preferably —OH. Y of R²² may be —SR^{Z}. Most preferably, for formula (I) and/or formula (II), R²² represents the following group:

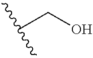 [SMILES: *CO]

R^{2b} may represent C4-10 alkyl, C4-10 alkenyl, C6-12 aryl, or C4-12 heterocyclyl, optionally substituted with one or more Y groups. Preferably R^{2b} represents C6-12 aryl, or C4-12 heterocyclyl, optionally substituted with one or more Y groups. More preferably R^{2b} represents C6-12 aryl optionally substituted with one or more Y groups, such as C6-8 aryl optionally substituted with one or more Y groups. Yet more preferably R^{2b} represents benzyl (i.e. —CH₂Ph), and is preferably substituted with one or more Y groups.

R^{2b} may be substituted with from 0 to 2 Y groups, preferably 1 Y group. Preferably R^{2b} represents C6-12 aryl or C4-12 heterocyclyl optionally substituted with one Y group, such as C6-8 aryl or C4-8 heterocyclyl optionally substituted with one Y group. R^{2b} may be substituted with one or more Y group(s) independently selected from the list consisting of —C(O)OR^{Z}, —C(O)NHR^{Z}, —S(O)₂NHR^{Z}, —NR^{Z}₂ and —OR^{Z}; preferably —NR^{Z}₂ and —OR^{Z}; most preferably —OR^{Z}. Y for R^{2b} may be —SR^{Z}. For R^{2b} preferably R^{Z} is H. As such, R^{2b} may be substituted with one or more Y group(s) independently selected from the list consisting of —C(O)OH, —C(O)NH₂, —S(O)₂NH₂, —NH₂ and —OH; preferably —NH₂ and —OH; more preferably —OH. More preferably R^{2b} represents benzyl substituted with one Y group at the para-position of the phenyl ring. Most preferably R^{2b} represents benzyl substituted with one —OH group, for example at the para-position of the phenyl ring:

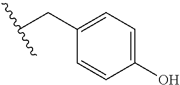 [SMILES: *Cc1ccc(O)cc1]

R^{2c} and/or R^{2h} may represent C4-10 alkyl, C4-10 alkenyl, C6-12 aryl, or C4-12 heterocyclyl, each optionally substituted with one or more Y groups. For example, R^{2c} and/or R^{2h} may represent C6-12 aryl, or C4-12 heterocyclyl, each optionally substituted with one or more Y groups. Preferably R^{2c} and/or R^{2h} represents C6-12 aryl, or C5-12 heteroaryl, each optionally substituted with one or more Y groups. Preferably R^{2c} and/or R^{2h} are unsubstituted. More preferably R^{2c} and/or R^{2h} represent a C6-10 heteroaryl group that contains from one to three nitrogen atoms, for example one or two nitrogen atoms, such as an indole. Most preferably R^{2c} and/or R^{2h} represent:

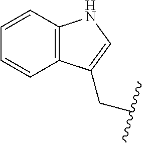 [SMILES: *Cc1c[nH]c2ccccc12]

From one to three of the groups R²² to R^{2l} may represent C4-10 alkyl, C4-10 alkenyl, C6-12 aryl, or C4-12 heterocyclyl, each optionally substituted with one Y group. For example, from one to three, preferably two, of the groups R^{2a} to R^{2l} may represent C6-12 aryl, or C4-12 heterocyclyl, each optionally substituted with one Y group. Preferably one to three, more preferably two, of the groups R^{2a} to R^{2l} represent a C6-10 heteroaryl group that contains from one to three nitrogen atoms, for example one or two nitrogen atoms, such as an indole, e.g:

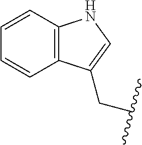 [SMILES: *Cc1c[nH]c2ccccc12]

R^{2d}, R^{2e} and/or R^{2g} may independently represent C1-10 alkyl, C2-10 alkenyl, C6-12 aryl, or C4-12 heterocyclyl, each substituted with from one to three Y groups represented by NR^{Z}₂, such as NH₂. Preferably two or, most preferably, three of the groups R^{2d}, R^{2e} and R^{2g} independently represent C1-10 alkyl, C2-10 alkenyl, C6-12 aryl, or C4-12 heterocyclyl, each substituted with from one to three Y groups represented by NR^{Z}₂, such as NH₂. More preferably R^{2d}, R^{2e} and/or R^{2g} (preferably two, and more preferably all three of these groups) independently represent C1-10 alkyl or C2-10 alkenyl, each substituted with from one to three Y groups represented by NR^{Z}₂, such as NH₂. For example R^{2d}, R^{2e} and/or R^{2g} may independently represent C1-6 alkyl or C2-6 alkenyl, each substituted with from one to three Y groups represented by NR^{Z}₂, such as NH₂. Preferably the number of Y groups represented by NR^{Z}₂, such as NH₂, is one or two, more preferably one. For example, R^{2d}, R^{2e} and/or R^{2g} (preferably two, and more preferably three of these groups) may represent C1-10 alkyl, C2-10 alkenyl, C6-12 aryl, or C4-12 heterocyclyl, each substituted with one or two, preferably one, Y groups represented by NR^{Z}₂, such as NH₂. Most preferably R^{2d}, R^{2e} and/or R^{2g} represents:

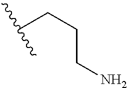 [SMILES: *CCCN]

R^{2f} and/or R^{2l} may represent H, C1-8 alkyl, C2-8 alkenyl, C6-8 aryl, or C4-8 heterocyclyl, each optionally substituted with one or more Y groups. It will be appreciated that in one embodiment R^{2f} and/or R^{2l} are not substituted. For example, R^{2f} and/or R^{2l} may represent H, C1-8 alkyl or C2-8 alkenyl each optionally substituted with one or more Y groups. Preferably R^{2f} and/or R^{2l} represents H, C1-8 alkyl or C2-8 alkenyl. For example, R^{2f} and/or R^{2l} may represent H, C1-4 alkyl or C2-4 alkenyl. Most preferably R^{2f} and/or R^{2h} represents H.

R²ⁱ may represent H, C1-8 alkyl, C2-8 alkenyl, C6-8 aryl, or C4-8 heterocyclyl, each optionally substituted with one or more Y groups. For example, R²ⁱ may represent H, C1-8 alkyl or C2-8 alkenyl each optionally substituted with one or more Y groups. Preferably R²ⁱ represents branched C1-8 alkyl or branched C2-8 alkenyl each optionally substituted with one or more Y groups. Preferably R²ⁱ represents H, C1-8 alkyl or C2-8 alkenyl. For example, R²ⁱ may represent H, C1-6 alkyl or C2-6 alkenyl. More preferably R²ⁱ represents C3, C4 or C5 alkyl or C3, C4 or C5 alkenyl. Preferably R²ⁱ represents branched C3, C4 or C5 alkyl or branched C3, C4 or C5 alkenyl. Most preferably R²ⁱ represents a 1-methylpropyl group:

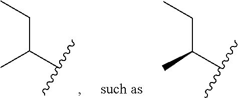

R^{2j} may represent H, C1-8 alkyl, C2-8 alkenyl, C6-8 aryl, or C4-8 heterocyclyl, each optionally substituted with one or more Y groups. R^{2j} preferably represents H, C1-4 alkyl or C2-4 alkenyl, more preferably H or C1-4 alkyl, each optionally substituted with one or more Y groups. Most preferably R^{2j} represents H, or C1 or C2 alkyl optionally substituted with one or more Y groups. R^{2j} may be substituted with one or more, preferably one, Y group represented by —C(O)OR^{Z}, —C(O)NHR^{Z}, —S(O)₂NHR^{Z}, —NR^{Z}₂ or —OR^{Z}. Y may be —SR^{Z}. R^{2j} may be substituted with one or more, preferably one, Y group preferably represented by —C(O)OH, —C(O)NH₂, —S(O)₂NH₂, —NH₂ or —OH. In one embodiment R^{2j} is not H.

R^{2j} of formulae (I) and/or (II) may represent C1-8 alkyl, C2-8 alkenyl, C6-8 aryl, or C4-8 heterocyclyl, each substituted with one Y group represented by —C(O)OR^{Z}, —C(O)NHR^{Z}, —S(O)₂NHR^{Z}, —NR^{Z}₂ or —OR^{Z}. For example, for formulae (I) and/or (II), R^{2j} may represent C1-8 alkyl or C2-8 alkenyl (e.g. C1-4 alkyl or C2-4 alkenyl) each substituted with one Y group represented by —C(O)OR^{Z}, —C(O)NHR^{Z}, —S(O)₂NHR^{Z}, —NR^{Z}₂ or —OR^{Z}. Y may be —SR^{Z}. For formulae (I) and/or (II), preferably, R^{2j} is substituted with one Y group represented by —C(O)OH, —C(O)NH₂, —S(O)₂NH₂, or —NHS(O)₂H. Preferably, for formulae (I) and/or (II), R^{2j} represents C1-4 alkyl, C2-4 alkenyl, each substituted with one Y group represented by —C(O)NHR^{Z}, such as —C(O)NH₂. For formulae (I) and/or (II), R^{2j} is preferably substituted with one Y group represented by —C(O)NH₂. Most preferably R^{2j} of formulae (I) and/or (II) represents the group:

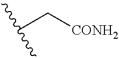 [SMILES: *CC(N)=O]

R^{2k} may represent H, C1-6 alkyl or C2-6 alkenyl, each optionally substituted with one or more Y group. It will be appreciated that in one embodiment R^{2k} is not substituted. R^{2k} preferably represents H, C1-4 alkyl or C2-4 alkenyl, more preferably H or C1-4 alkyl, each optionally substituted with one or more Y group. Most preferably R^{2k} represents H, C1 or C2 alkyl optionally substituted with one or more Y group. R^{2k} may be substituted with one Y group. For R^{2k}, Y may be represented by —C(O)OR^{Z}, —C(O)NHR^{Z}, —S(O)₂NHR^{Z}, —NR^{Z}₂ or —OR^{Z}. Y may be —SR^{Z}. R^{2k} is preferably substituted with a Y group represented by —C(O)OH, —C(O)NH₂, —S(O)₂NH₂, —NH₂ or —OH. R^{2k} may represent H, C1-8 alkyl, C2-8 alkenyl, C6-8 aryl, or C4-8 heterocyclyl, each optionally substituted with one or more Y groups. For example, R^{2k} may represent H, C1-8 alkyl or C2-8 alkenyl each optionally substituted with one or more Y groups. Preferably, R^{2k} represents H, C1-8 alkyl or C2-8 alkenyl, for example H, C1-4 alkyl or C2-4 alkenyl, or H or C1-2 alkyl. Most preferably, R^{2k} represents H.

R³ has been shown to exhibit particularly high tolerance to substitution with other functionality. This group may represent H, C1-8 alkyl, C2-8 alkenyl, C6-8 aryl, or C4-8 heterocyclyl, each optionally substituted with one or more Y groups, for example H, C1-8 alkyl, or C2-8 alkenyl, each optionally substituted with one Y group.

R³ may be substituted with one Y group. In embodiment R³ represents H, C1-8 alkyl or C2-8 alkenyl substituted with one Y group.

For R³, Y may represent cyano, halogen, N₃, —C(O)R^{Z}, —C(O)OR^{Z}, —OC(O)R^{Z}, —C(O)NHR^{Z}, —NHC(O)R^{Z}, —NHC(O)NHR^{Z}, —NHC(O)OR^{Z}, —OC(O)NHR^{Z}, OS(O)₂R^{Z}, —S(O)₂NHR^{Z}, —NHS(O)₂R^{Z}, —NR^{Z}₂ or —OR^{Z}. Y may be —NHC(NH)NHR^{Z}, or —SR^{Z}. Preferably, for R³, Y represents cyano, N₃, —C(O)OR^{Z}, —OC(O)R^{Z}, —C(O)NHR^{Z}, —NHC(O)R^{Z}, —NHC(O)NHR^{Z}, —NHC(O)OR^{Z}, —OC(O)NHR^{Z}, —NR^{Z}₂ or —OR^{Z}. Also, preferably Y is —NHC(NH)NHR^{Z}, or —SR^{Z}. More preferably, for R³, Y represents N₃, —C(O)OR^{Z}, C(O)NHR^{Z}, —NHC(O)OR^{Z}, —NR^{Z}₂, or —OR^{Z}. Y may more preferably be —NHC(NH)NHR^{Z}, or —SR^{Z}. For example, R³ may be substituted by one Y group representing N₃, —C(O)OH, C(O)NH₂, —NHC(O)OR(C1-4 alkyl or C2-4 alkenyl), —NH₂ or —OH. More preferably Y is-NHC(NH)NH₂, or —SH. It is more preferred that R³ represents one of the following groups:

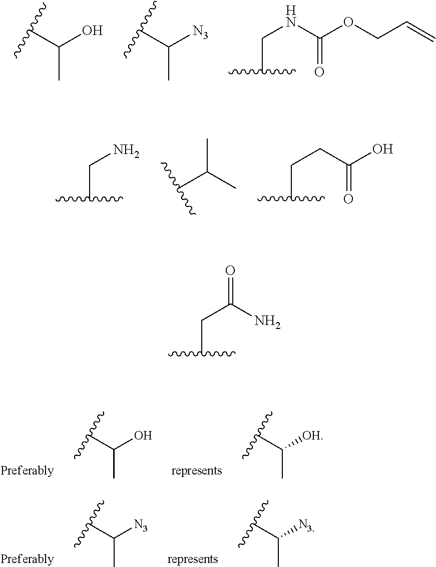

It is also more preferred that R³ represents one of the following groups: Bn, —(CH₂)₂CO₂(Allyl), —CH₂OH, —CH₂(CH₃)₂, —(CH₂)₂CONH₂,

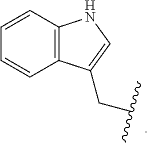 [SMILES: *Cc1c[nH]c2ccccc12]

In another embodiment R³ represents H, C1-8 alkyl or C2-8 alkenyl, such as H, C1-4 alkyl or C2-4 alkenyl, more preferably H or C1-3 alkyl, yet more preferably H or C1 alkyl (i.e. methyl).

Each R^{Z} may independently represent H, C1-8 alkyl, C2-8 alkenyl, C6-8 aryl, or C4-8 heterocyclyl, for example H, C1-6 alkyl, C2-6 alkenyl, C6-8 aryl, or C4-6 heterocyclyl. Preferably each R^{Z} independently represents H, C1-10 alkyl or C2-10 alkenyl, such as H, C1-8 alkyl or C2-8 alkenyl. For example, each R^{Z} may independently represent H, C1-6 alkyl or C2-6 alkenyl, or H, C1-4 alkyl or C2-4 alkenyl.

X preferably represents NH or O. It is more preferred that X represents NH as this functionality can form, with an adjacent carbonyl group, part of amide group that provides a bond that is typically more stable to hydrolysis than that of an ester or a thioester, as are present where X is either O or S respectively. Preferably XR^{Z} represents NH₂, SH or OH, more preferably NH₂ or OH, most preferably NH₂.

The compounds of the present disclosure include an N-oxide or a pharmaceutically acceptable salt or a pharmaceutically acceptable solvate thereof. The synthesis of and use of N-oxides, pharmaceutically acceptable salts and pharmaceutically acceptable solvates will be well understood by the skilled person.

It will be understood that, for the most part, Y groups have been grouped together for conciseness and that, if required, one or more instances of Y may be limited independently of other Y groups. In this case the independent limited groups will be known as Y¹, Y² and so on.

In one preferred embodiment of the compounds of the present disclosure:
R¹ represents C6-12 alkyl, C6-12 alkenyl or C6-12 aryl, each optionally substituted with one —OH group,
R^{2a} to R^{2l} each independently represents H, C1-6 alkyl, C2-6 alkenyl, C6-12 aryl, C4-12 heterocyclyl, each optionally substituted with one Y group,
R³ represents H, C1-6 alkyl, C2-6 alkenyl, C6-12 aryl, C4-12 heterocyclyl, each optionally substituted with one Y group,
Each R^{Z} represents H, C1-6 alkyl, C2-6 alkenyl, C6-8 aryl, or C4-6 heterocyclyl, and
X represents NH or O.

In another preferred embodiment of the compounds of the present disclosure:
R^{2g} represents C1-10 alkyl, C2-10 alkenyl, C6-12 aryl, or C4-12 heterocyclyl, substituted with from one to three Y groups represented by NR^{Z}₂, such as NH₂,
R^{2h} represents C4-10 alkyl, C4-10 alkenyl, C6-12 aryl, or C4-12 heterocyclyl, optionally substituted with one or more Y groups, and
the other groups are as defined in the statement of invention relating to each aspect.

In a more preferred embodiment of the compounds of the present disclosure:
R^{2g} represents C1-10 alkyl or C2-10 alkenyl substituted with from one to three Y groups represented by NR^{Z}₂, such as NH₂,
R^{2h} represents C6-12 aryl or C4-12 heterocyclyl, optionally substituted with one or more Y groups, and
the other groups are as defined in the statement of invention relating to each aspect.

In another preferred embodiment of the compounds of the present disclosure:
R^{2d}, R^{2e} and R^{2g} each independently represent C1-10 alkyl, C2-10 alkenyl, C6-12 aryl, or C4-12 heterocyclyl, substituted with from one to three Y groups represented by NR^{Z}₂, such as NH₂,
R^{2h} represents C4-10 alkyl, C4-10 alkenyl, C6-12 aryl, or C4-12 heterocyclyl, optionally substituted with one or more Y groups, and
the other groups are as defined in the statement of invention relating to each aspect.

In another more preferred embodiment of the compounds of the present disclosure:
R^{2d}, R^{2e} and R^{2g} each independently represent C1-10 alkyl or C2-10 alkenyl substituted with from one to three Y groups represented by NR^{Z}₂, such as NH₂,
R^{2h} represents C6-12 aryl or C4-12 heterocyclyl, optionally substituted with one or more Y groups, and
the other groups are as defined in the statement of invention relating to each aspect.

In another preferred embodiment of the compounds of the present disclosure:
R^{2b} represents C4-10 alkyl, C4-10 alkenyl, C6-12 aryl, or C4-12 heterocyclyl, each optionally substituted with one or more Y groups,
R^{2d}, R^{2e} and R^{2g} each independently represent C1-10 alkyl, C2-10 alkenyl, C6-12 aryl, or C4-12 heterocyclyl, substituted with from one to three Y groups represented by NR^{Z}₂, such as NH₂,
R^{2c} and R^{2h} each independently represent C4-10 alkyl, C4-10 alkenyl, C6-12 aryl, or C4-12 heterocyclyl, optionally substituted with one or more Y groups,
R²ⁱ represents C3, C4 or C5 alkyl or C3, C4 or C5 alkenyl,
R^{2j} represents C1-8 alkyl, C2-8 alkenyl, C6-8 aryl, or C4-8 heterocyclyl, each substituted with one or more Y group, and
the other groups are as defined in the statement of invention relating to each aspect.

In another more preferred embodiment of the compounds of the present disclosure:
R^{2b} represents C6-12 aryl, or C4-12 heterocyclyl, substituted with one or more Y groups,
R^{2d}, R^{2e} and R^{2g} each independently represent C1-10 alkyl or C2-10 alkenyl substituted with from one to three Y groups represented by NR^{Z}₂, such as NH₂,
R^{2c} and R^{2h} each independently represent C6-12 aryl or C4-12 heterocyclyl, optionally substituted with one or more Y groups,
R²ⁱ represents C3, C4 or C5 alkyl or C3, C4 or C5 alkenyl,
R^{2j} represents C1-8 alkyl, C2-8 alkenyl, C6-8 aryl, or C4-8 heterocyclyl, each substituted with one Y group represented by —C(O)OR^{Z}, —C(O)NHR^{Z}, —S(O)₂NHR^{Z}, —NR^{Z}₂ or —OR^{Z}, and
the other groups are as defined in the statement of invention relating to each aspect.

In another more preferred embodiment of the compounds of the present disclosure:
R^{2b} represents C6-12 aryl, or C4-12 heterocyclyl, substituted with one or more Y groups,
R^{2d}, R^{2e} and R^{2g} each independently represent C1-10 alkyl or C2-10 alkenyl substituted with from one to three Y groups represented by NR^{Z}₂, such as NH₂,
R^{2c} and R^{2h} each independently represent C6-12 aryl or C4-12 heterocyclyl, optionally substituted with one or more Y groups,
R²ⁱ represents C3, C4 or C5 alkyl or C3, C4 or C5 alkenyl,
R^{2j} represents C1-8 alkyl, C2-8 alkenyl, C6-8 aryl, or C4-8 heterocyclyl, each substituted with one Y group represented by —C(O)OR^{Z}, —C(O)NHR^{Z}, —S(O)₂NHR^{Z}, —NR^{Z}₂ or —OR^{Z},
R³ represents H, C1-6 alkyl, C2-6 alkenyl, C6-12 aryl, C6-12 heterocyclyl, optionally substituted with one Y group, and
the other groups are as defined in the statement of invention relating to each aspect.

In another more preferred embodiment of the compounds of the present disclosure:
R¹ represents C6-12 alkyl, C6-12 alkenyl or C6-12 aryl, each optionally substituted with one Y group represented by —NR^{Z}₂ or —OR^{Z} or optionally —SR^{Z},
R^{2a} represents C1-4 alkyl or C2-4 alkenyl, each optionally substituted with one Y group represented by —C(O)OR^{Z}, —C(O)NHR^{Z}, —S(O)₂NHR^{Z}, —NR^{Z}₂ or —OR^{Z}, or optionally —NHC(NH)NHR^{Z} or —SR^{Z},
R^{2b} represents C6-10 aryl, or C4-10 heterocyclyl, each optionally substituted with one Y group represented by —NR^{Z}₂ or —OR^{Z} or optionally —SR^{z}.
R^{2c} represents C6-12 aryl, or C4-12 heteroaryl, each optionally substituted with one Y group,
R^{2d} represents C1-6 alkyl or C2-6 alkenyl, each optionally substituted with one Y group represented by —NR^{Z}₂ or —OR^{z} or optionally —SR^{z},
R^{2e} represents C1-6 alkyl or C2-6 alkenyl, each optionally substituted with one Y group represented by —NR^{Z}₂ or —OR^{z} or optionally —SR^{z},
R^{2f} represents H, C1-4 alkyl or C2-4 alkenyl,
R^{2g} represents C1-6 alkyl or C2-6 alkenyl, each optionally substituted with one Y group represented by —NR^{Z}₂ or —OR^{z} or optionally —SR^{z},
R^{2h} represents C6-12 aryl, or C4-12 heteroaryl, each optionally substituted with one Y group,
R²ⁱ represents H, C1-6 alkyl or C2-6 alkenyl,
R^{2j} represents H, C1-4 alkyl or C2-4 alkenyl, each optionally substituted with one Y group represented by —C(O)OR^{Z}, —C(O)NHR^{Z}, —S(O)₂NHR^{z}, —NR^{Z}₂ or —OR^{Z} or optionally —NHC(NH)NHR^{Z}, or —SR^{Z},
Any R^{2k} represents H, C1-4 alkyl or C2-4 alkenyl, each optionally substituted with one Y group represented by —NR^{Z}₂ or —OR^{z} or optionally —SR^{z},
Any R^{2l} represents H, C1-4 alkyl or C2-4 alkenyl,
R³ represents H, C1-6 alkyl, C2-6 alkenyl, C6-12 aryl, C6-12 heterocyclyl, optionally substituted with one Y group,
Each R^{Z} independently represents H, C1-6 alkyl or C2-6 alkenyl, and
X represents NH or O.

In a yet more preferred embodiment of the compounds of the present disclosure:
R¹ represents C6-12 alkyl, C6-12 alkenyl or C6-12 aryl,
R^{2a} represents C1-4 alkyl or C2-4 alkenyl, each preferably substituted with one Y group represented by —C(O)OH, —C(O)NH₂, —S(O)₂NH₂, —NH₂ or —OH or optionally —NHC(NH)NH₂ or —SH,
R^{2b} represents C6-10 aryl, or C4-10 heterocyclyl, preferably substituted with one Y group represented by —NH₂ or —OH or optionally —SH.
R^{2c} represents C6-12 aryl, or C4-12 heteroaryl,
R^{2d} represents C1-6 alkyl or C2-6 alkenyl, each preferably substituted with one Y group represented by —NH₂ or —OH or optionally —SH,
R^{2e} represents C1-6 alkyl or C2-6 alkenyl, each preferably substituted with one Y group represented by —NH₂ or —OH or optionally —SH,
R^{2f} represents H or C1-2 alkyl,
R^{2g} represents C1-6 alkyl or C2-6 alkenyl, each preferably substituted with one Y group represented by —NH₂ or —OH or optionally —SH,
R^{2h} represents C6-12 aryl, or C4-12 heteroaryl,
R²ⁱ represents branched C3-5 alkyl or branched C3-5 alkenyl,
R^{2j} represents H, C1-4 alkyl or C2-4 alkenyl, each optionally substituted with one Y group represented by —C(O)OH or —C(O)NH₂,
Any R^{2k} represents H, C1-4 alkyl or C2-4 alkenyl, each optionally substituted with one Y group represented by —NH₂ or —OH or optionally —SH,
Any R^{2l} represents H or C1-2 alkyl,
R³ represents H, C1-4 alkyl, C2-4 alkenyl, optionally substituted with one Y group represented by N₃, —C(O)OR^{Z}, —OC(O)R^{Z}, —C(O)NHR^{Z}, —NHC(O)R^{Z}, —NHC(O)NHR^{Z}, —NHC(O)OR^{Z}, OC(O)NHR^{Z}, —NR^{Z}₂ or —OR^{z}, or optionally —NHC(NH)NHR^{Z} or —SR^{Z},
Each R^{Z} independently represents H, C1-4 alkyl or C2-4 alkenyl, and
X represents NH or O, preferably NH.

In another preferred embodiment of the compounds of the present disclosure, especially of formulae (I) and/or (II):
R¹ represents C6-12 alkyl,
R^{2a} represents C1-4 alkyl substituted with one Y group represented by —NH₂ or —OH or optionally —SH,
R^{2b} represents C6-10 aryl substituted with one Y group represented by —NH₂ or —OH or optionally —SH,
R^{2c} represents C6-12 aryl, or C4-12 heteroaryl,
R^{2d} represents C1-6 alkyl substituted with one Y group represented by —NH₂,
R^{2e} represents C1-6 alkyl substituted with one Y group represented by —NH₂,
R^{2f} represents H or C1-2 alkyl,
R^{2g} represents C1-6 alkyl substituted with one Y group represented by —NH₂,
R^{2h} represents C6-12 aryl, or C4-12 heteroaryl,
R²ⁱ represents branched C3-5 alkyl or branched C3-5 alkenyl,
R^{2j} represents H, C1-4 alkyl substituted with one Y group represented by —C(O)NH₂,
Any R^{2k} represents H or C1-2 alkyl,
Any R^{2l} represents H or C1-2 alkyl,
R³ represents H, C1-4 alkyl, C2-4 alkenyl, optionally substituted with one Y group represented by N₃, —C(O)OR^{Z}, —OC(O)R^{Z}, —C(O)NHR^{Z}, —NHC(O)R^{Z}, —NHC(O)NHR^{Z}, —NHC(O)OR^{Z}, OC(O)NHR^{Z}, —NR^{Z}₂ or —OR^{Z}, or optionally —NHC(NH)NHR^{Z} or —SR^{Z},
Each R^{Z} independently represents H, C1-4 alkyl or C2-4 alkenyl, and
X represents NH.

The compounds of the present disclosure are not limited to any particular stereoisomer. The compounds include tautomeric or stereochemically isomeric forms thereof. However, in one embodiment, the stereochemical configuration of the amino acid backbone and/or the isoleucine group R²ⁱ of laterocidine, as shown in FIG. 1, is used for compounds of Formulae (I) and/or (II).

Preferably formula (I) represents the following structure:

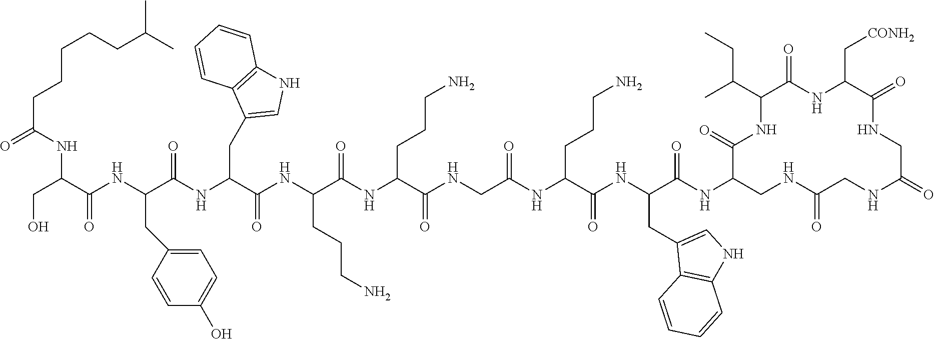 [SMILES: CCC(C)C1NC(=O)C(NC(=O)C(Cc2c[nH]c3ccccc23)NC(=O)C(CCCN)NC(=O)CNC(=O)C(CCCN)NC(=O)C(CCCN)NC(=O)C(Cc2c[nH]c3ccccc23)NC(=O)C(Cc2ccc(O)cc2)NC(=O)C(CO)NC(=O)CCCCCC(C)C)CNC(=O)CNC(=O)CNC(=O)C(CC(N)=O)NC1=O]

More preferably formula (I) represents laterocidamide, which has the following structure:

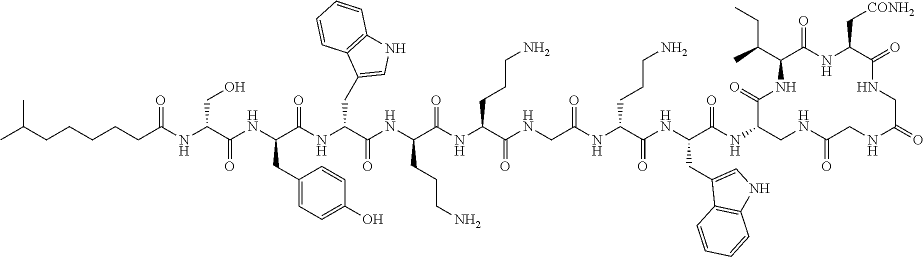 [SMILES: CC[C@H](C)[C@@H]1NC(=O)[C@@H](NC(=O)[C@H](Cc2c[nH]c3ccccc23)NC(=O)[C@@H](CCCN)NC(=O)CNC(=O)[C@H](CCCN)NC(=O)[C@@H](CCCN)NC(=O)[C@@H](Cc2c[nH]c3ccccc23)NC(=O)[C@@H](Cc2ccc(O)cc2)NC(=O)[C@@H](CO)NC(=O)CCCCCC(C)C)CNC(=O)CNC(=O)CNC(=O)[C@H](CC(N)=O)NC1=O]

Preferably formula (II) represents the structure:

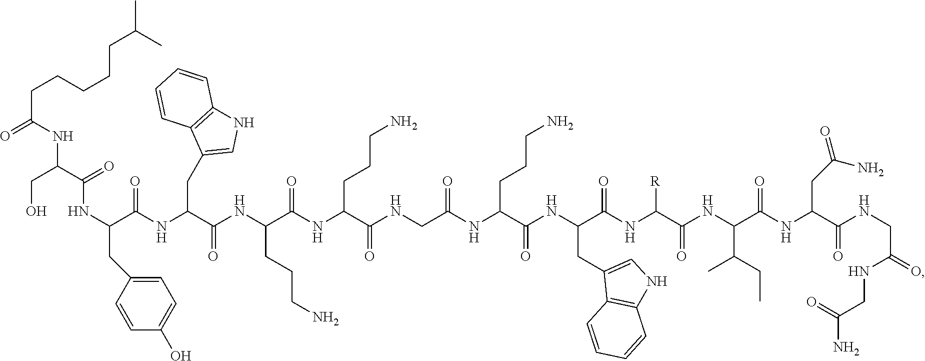

for example

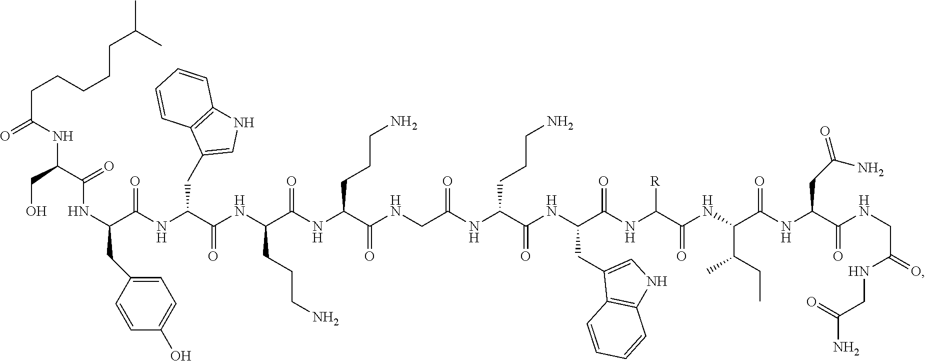

where R represents either:

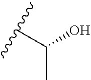 [SMILES: *[C@@H](C)O]

(forming a Thr residue),

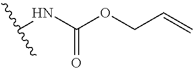 [SMILES: *NC(=O)OCC=C]

(forming a Dap(Alloc) residue), or

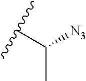 [SMILES: *[C@@H](C)N=[N+]=[N-]]

forming a (2S,3R)-2-amino-3-azidobutanoic acid residue).

Optionally formula (I) excludes the following structure, which it will be appreciated represents all stereochemical isomers of laterocidine:

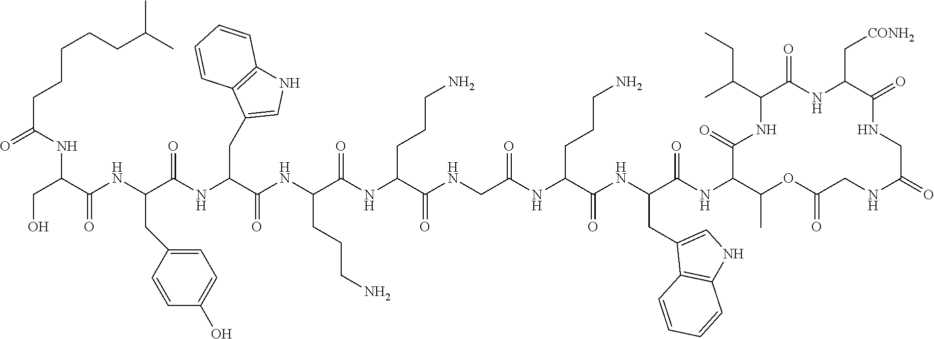 [SMILES: CCC(C)C1NC(=O)C(NC(=O)C(Cc2c[nH]c3ccccc23)NC(=O)C(CCCN)NC(=O)CNC(=O)C(CCCN)NC(=O)C(CCCN)NC(=O)C(Cc2c[nH]c3ccccc23)NC(=O)C(Cc2ccc(O)cc2)NC(=O)C(CO)NC(=O)CCCCCC(C)C)C(C)OC(=O)CNC(=O)CNC(=O)C(CC(N)=O)NC1=O]

In one embodiment formula (I) excludes the enantiomer of one or both of relacidine A and relacidine B. Preferably formula (I) excludes one or both of the following structures, which it will be appreciated represent all stereochemical isomers of relacidine A and relacidine B respectively:

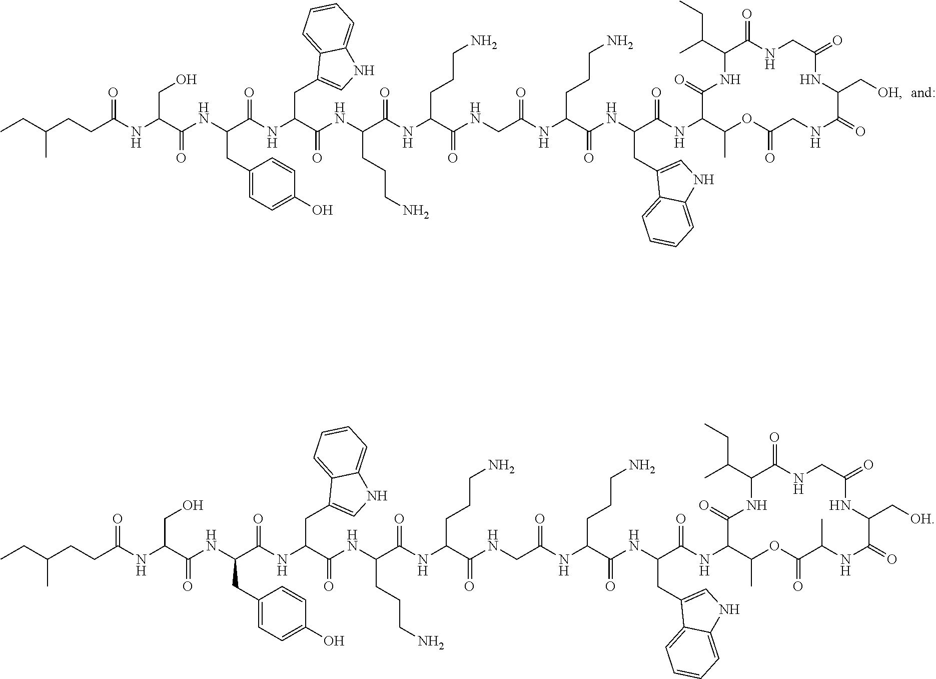

It will be appreciated that the compounds of the invention are based upon natural products. In one embodiment the R¹ and/or R³ groups of the compounds of the invention exclude the group corresponding to laterocidine, brevicidine and/or relacidine. In one embodiment one or all of the R groups of the compounds of the invention exclude the group corresponding to laterocidine, brevicidine and/or relacidine. Such groups are described as the most preferable groups above. For example, R^{2a} of the compound of the invention may not correspond to —CH₂OH, as found in laterocidine.

Further Compounds

According to a seventh aspect, the present invention provides a compound of formula (III) or (IV):

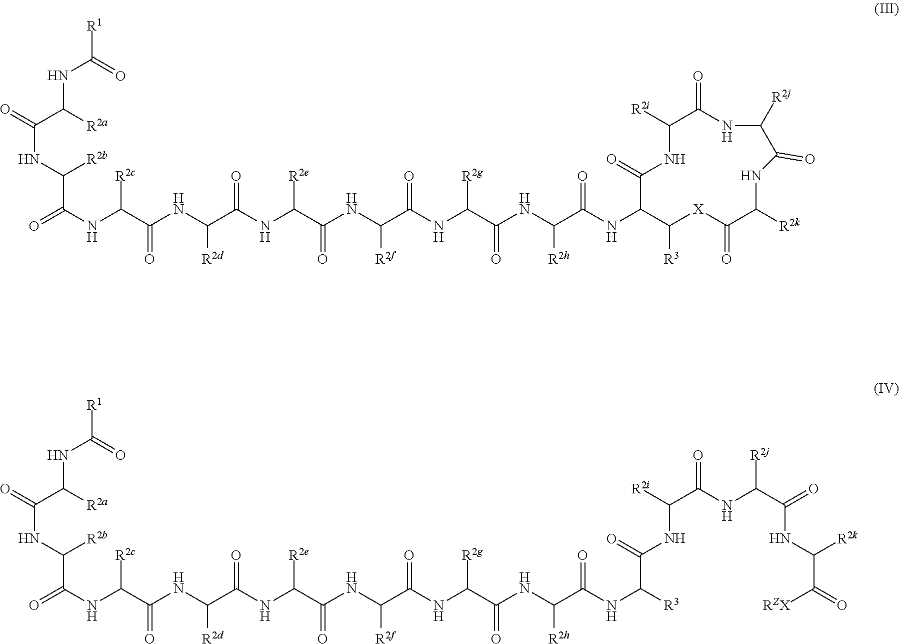

including tautomeric or stereochemically isomeric forms thereof, wherein:
R¹ represents C1-20 alkyl, C2-20 alkenyl, C6-20 aryl, or C4-20 heterocyclyl, each optionally substituted with one or more Y groups,
R^{2a} to R^{2k} each independently represents H, C1-10 alkyl, C2-10 alkenyl, C6-12 aryl, or C4-12 heterocyclyl, each optionally substituted with one or more Y groups,
R³ represents H, C1-10 alkyl, C2-10 alkenyl, C6-12 aryl, or C4-12 heterocyclyl, each optionally substituted with one or more Y groups,
X represents NH, S or O,
each Y independently represents cyano, halogen, —C(O)R^{Z}, —C(O)OR^{Z}, —OC(O)R^{Z}, —C(O)NHR^{Z}, —NHC(O)R^{Z}, —NHC(O)NHR^{Z}, —NHC(NH)NHR^{Z}, —NHC(O)OR^{Z}, —OC(O)NHR^{Z}, —OS(O)₂R^{Z}, —S(O)₂NHR^{Z}, —NHS(O)₂R^{Z}, —SR^{Z}, —NR^{Z}₂ or —OR^{Z}, and
each R^{Z} independently represents H, C1-10 alkyl, C2-10 alkenyl, C6-10 aryl, or C4-1N heterocyclyl;
or an N-oxide thereof or a pharmaceutically acceptable salt thereof or a pharmaceutically acceptable solvate thereof;
   and wherein formula (III) excludes brevicidine:

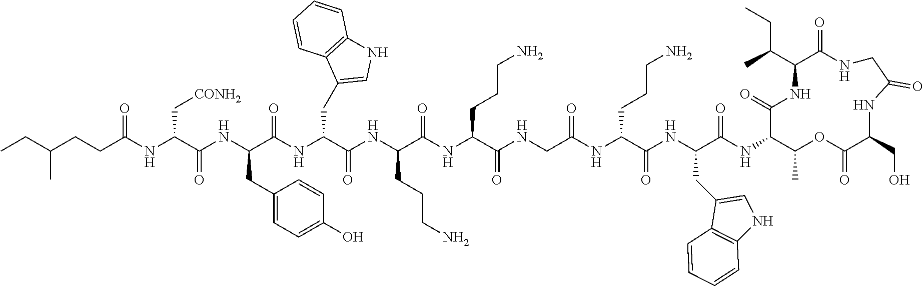 [SMILES: CCC(C)CCC(=O)N[C@H](CC(N)=O)C(=O)N[C@H](Cc1ccc(O)cc1)C(=O)N[C@H](Cc1c[nH]c2ccccc12)C(=O)N[C@H](CCCN)C(=O)N[C@@H](CCCN)C(=O)NCC(=O)N[C@H](CCCN)C(=O)N[C@@H](Cc1c[nH]c2ccccc12)C(=O)N[C@@H]1C(=O)N[C@@H]([C@@H](C)CC)C(=O)NCC(=O)N[C@@H](CO)C(=O)O[C@@H]1C]

Formula (III) may exclude the following formula:

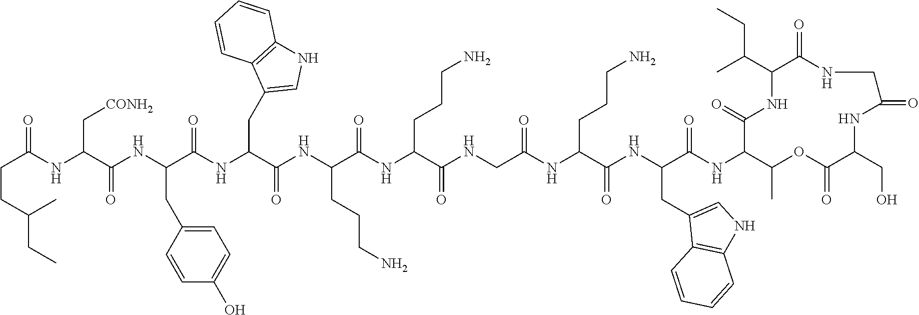 [SMILES: CCC(C)CCC(=O)NC(CC(N)=O)C(=O)NC(Cc1ccc(O)cc1)C(=O)NC(Cc1c[nH]c2ccccc12)C(=O)NC(CCCN)C(=O)NC(CCCN)C(=O)NCC(=O)NC(CCCN)C(=O)NC(Cc1c[nH]c2ccccc12)C(=O)NC1C(=O)NC(C(C)CC)C(=O)NCC(=O)NC(CO)C(=O)OC1C]

Formula (III) may be represented by:

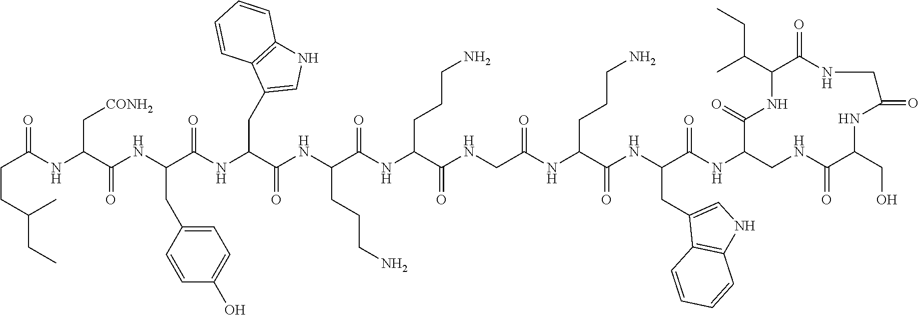 [SMILES: CCC(C)CCC(=O)NC(CC(N)=O)C(=O)NC(Cc1ccc(O)cc1)C(=O)NC(Cc1c[nH]c2ccccc12)C(=O)NC(CCCN)C(=O)NC(CCCN)C(=O)NCC(=O)NC(CCCN)C(=O)NC(Cc1c[nH]c2ccccc12)C(=O)NC1CNC(=O)C(CO)NC(=O)CNC(=O)C(C(C)CC)NC1=O]

Formula (III) may be represented by brevicidamide:

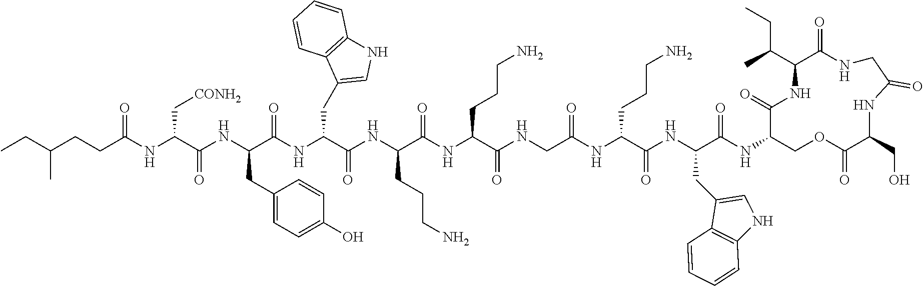 [SMILES: CCC(C)CCC(=O)N[C@H](CC(N)=O)C(=O)N[C@H](Cc1ccc(O)cc1)C(=O)N[C@H](Cc1c[nH]c2ccccc12)C(=O)N[C@H](CCCN)C(=O)N[C@@H](CCCN)C(=O)NCC(=O)N[C@H](CCCN)C(=O)N[C@@H](Cc1c[nH]c2ccccc12)C(=O)N[C@H]1COC(=O)[C@H](CO)NC(=O)CNC(=O)[C@H]([C@@H](C)CC)NC1=O]

According to an eighth aspect, the present invention provides a compound of formula (V):

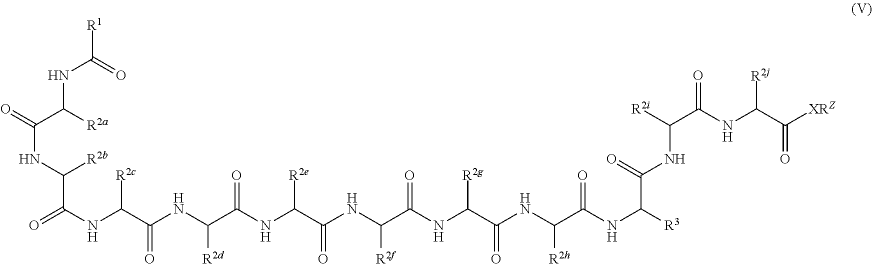

including tautomeric or stereochemically isomeric forms thereof, wherein:
R¹ represents C1-20 alkyl, C2-20 alkenyl, C6-20 aryl, or C4-20 heterocyclyl, each optionally substituted with one or more Y groups,
R^{2a} to R^{2j} each independently represents H, C1-10 alkyl, C2-10 alkenyl, C6-12 aryl, or C4-12 heterocyclyl, each optionally substituted with one or more Y groups,
R³ represents H, C1-10 alkyl, C2-10 alkenyl, C6-12 aryl, or C4-12 heterocyclyl, each optionally substituted with one or more Y groups,
X independently represents NH, S or O,
each Y independently represents cyano, halogen, N₃, —C(O)R^{Z}, —C(O)OR^{Z}, —OC(O)R^{Z}, —C(O)NHR^{Z}, —NHC(O)R^{Z}, —NHC(O)NHR^{Z}, —NHC(NH)NHR^{Z}, —NHC(O)OR^{Z}, —OC(O)NHR^{Z}, —OS(O)₂R^{Z}, —S(O)₂NHR^{Z}, —NHS(O)₂R^{Z}, —SR^{Z}, —NR^{Z}₂ or —OR^{Z}, and
each R^{Z} independently represents H, C1-10 alkyl, C2-10 alkenyl, C6-10 aryl, or C4-10 heterocyclyl,
or an N-oxide thereof or a pharmaceutically acceptable salt thereof or a pharmaceutically acceptable solvate thereof.

Formula (V) may be represented by the following structure:

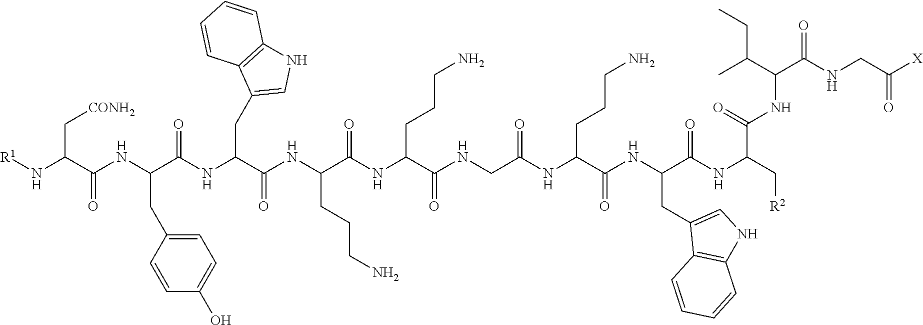

or, preferably:

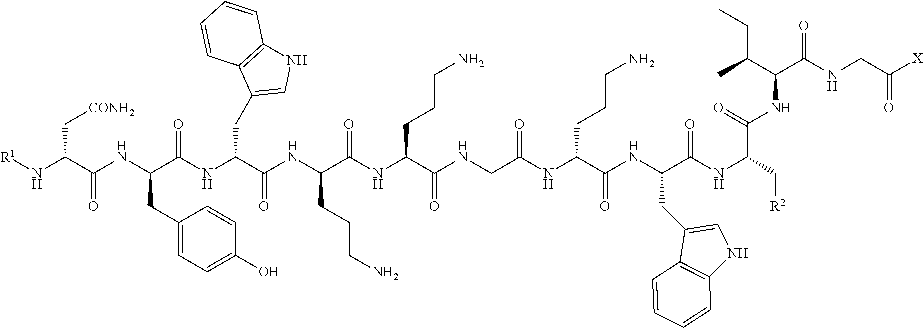

wherein:
R¹ represents C1-C20 alkyl, such as octanoyl or 4-methylhexanoyl,
R² represents

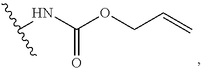 [SMILES: *NC(=O)OCC=C]

NH₂, OH,

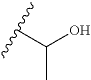 [SMILES: *C(C)O]

or H, and
X represents OH or NH₂.

For the preceding structure, the following combinations of groups are preferable:

| Compound | R¹ | R² | X |
|---|---|---|---|
| 1 | 4-methylhexanoyl | 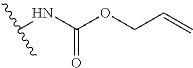 [SMILES: *NC(=O)OCC=C] | OH |
| 2 | octanoyl | 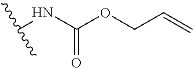 [SMILES: *NC(=O)OCC=C] | OH |
| 3 | 4-methylhexanoyl | H | OH |
| 4 | octanoyl | H | OH |
| 5 | 4-methylhexanoyl | 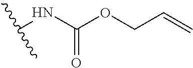 [SMILES: *NC(=O)OCC=C] | NH₂ |
| 6 | octanoyl | 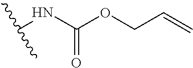 [SMILES: *NC(=O)OCC=C] | NH₂ |
| 7 | 4-methylhexanoyl | NH₂ | NH₂ |
| 8 | 4-methylhexanoyl | OH | NH₂ |
| 9 | 4-methylhexanoyl | 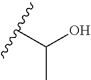 [SMILES: *C(C)O] | NH₂ |

Preferably

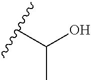 [SMILES: *C(C)O]

represents

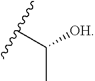 [SMILES: *[C@@H](C)O]

According to a ninth aspect, the present invention provides a compound of formula (VI):

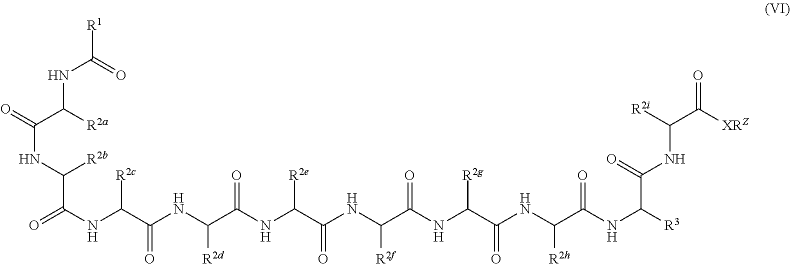

including tautomeric or stereochemically isomeric forms thereof, wherein:
R¹ represents C1-20 alkyl, C2-20 alkenyl, C6-20 aryl, or C4-20 heterocyclyl, each optionally substituted with one or more Y groups,
R^{2a} to R²ⁱ each independently represents H, C1-10 alkyl, C2-10 alkenyl, C6-12 aryl, or C4-12 heterocyclyl, each optionally substituted with one or more Y groups,
R³ represents H, C1-10 alkyl, C2-10 alkenyl, C6-12 aryl, or C4-12 heterocyclyl, each optionally substituted with one or more Y groups,
X independently represents NH, S or O,
each Y independently represents cyano, halogen, N₃, —C(O)R^{Z}, —C(O)OR^{Z}, —OC(O)R^{Z}, —C(O)NHR^{Z}, —NHC(O)R^{Z}, —NHC(O)NHR^{Z}, —NHC(NH)NHR^{Z}, —NHC(O)OR^{Z}, —OC(O)NHR^{Z}, —OS(O)₂R^{Z}, —S(O)₂NHR^{z}, —NHS(O)₂R^{Z}, —NR^{Z}₂, —SR^{Z}, or —OR^{Z}, and
each R^{Z} independently represents H, C1-10 alkyl, C2-10 alkenyl, C6-10 aryl, or C4-10 heterocyclyl,
or an N-oxide thereof or a pharmaceutically acceptable salt thereof or a pharmaceutically acceptable solvate thereof.

Formula (VI) may be represented by the following structure:

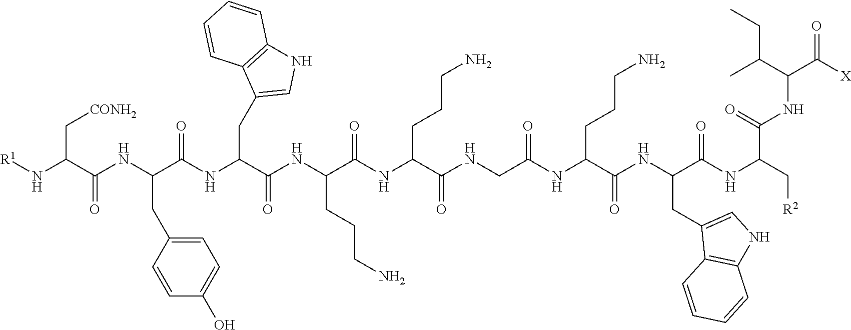

or, preferably:

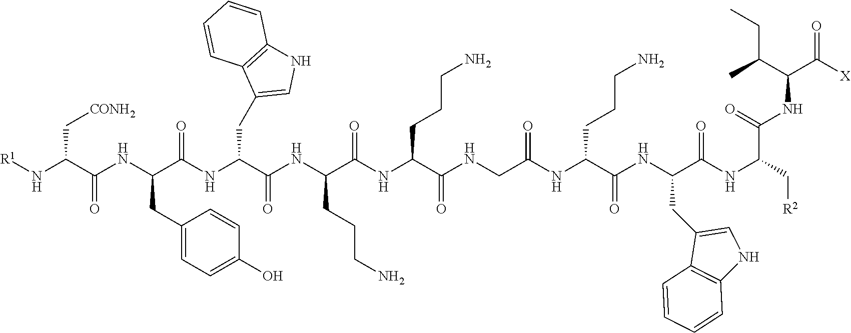

wherein:
R¹ represents C1-C20 alkyl, such as octanoyl or 4-methylhexanoyl,
R² represents

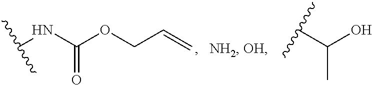

or H, and
X represents OH or NH₂.

According to a tenth aspect, the present invention provides a compound of formula (VII):

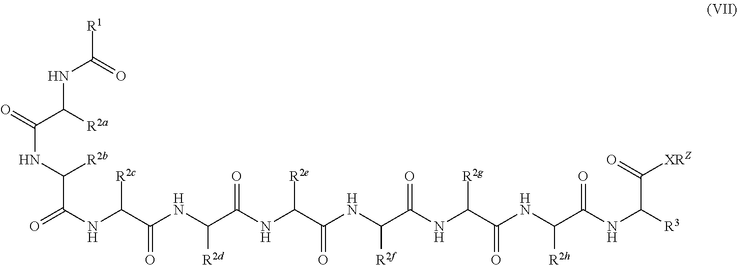

including tautomeric or stereochemically isomeric forms thereof, wherein:
R¹ represents C1-20 alkyl, C2-20 alkenyl, C6-20 aryl, or C4-20 heterocyclyl, each optionally substituted with one or more Y groups,
R^{2a} to R^{2h} each independently represents H, C1-10 alkyl, C2-10 alkenyl, C6-12 aryl, or C4-12 heterocyclyl, each optionally substituted with one or more Y groups,
R³ represents H, C1-10 alkyl, C2-10 alkenyl, C6-12 aryl, or C4-12 heterocyclyl, each optionally substituted with one or more Y groups,
X independently represents NH, S or O,
each Y independently represents cyano, halogen, N₃, —C(O)R^{Z}, —C(O)OR^{Z}, —OC(O)R^{Z}, —C(O)NHR^{Z}, —NHC(O)R^{Z}, —NHC(O)NHR^{Z}, —NHC(NH)NHR^{Z}, —NHC(O)OR^{Z}, —OC(O)NHR^{Z}, —OS(O)₂R^{Z}, —S(O)₂NHR^{z}, —NHS(O)₂R^{Z}, —SR^{Z}, —NR^{Z}₂ or —OR^{Z}, and
each R^{Z} independently represents H, C1-10 alkyl, C2-10 alkenyl, C6-10 aryl, or C4-10 heterocyclyl,
or an N-oxide thereof or a pharmaceutically acceptable salt thereof or a pharmaceutically acceptable solvate thereof.

Formula (VII) may be represented by the following structure:

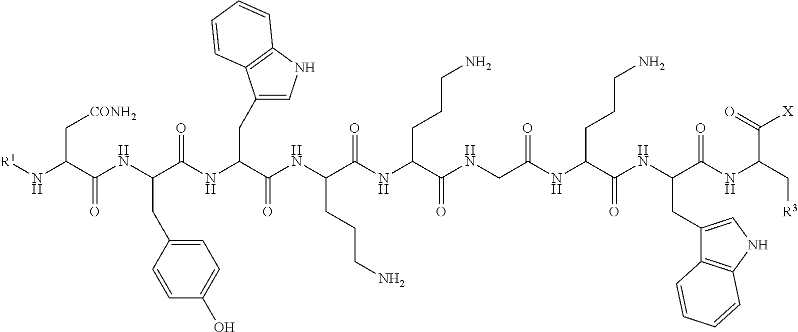

or, preferably:

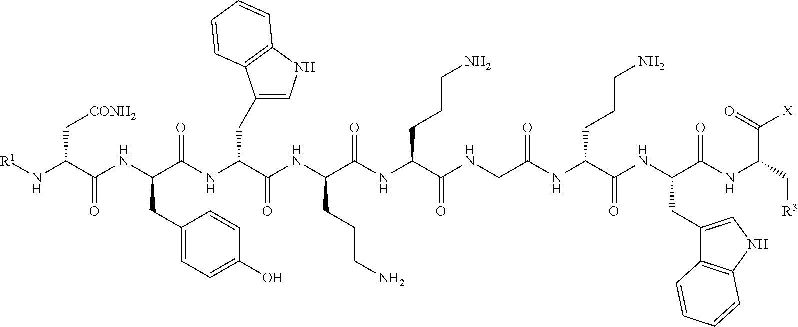

wherein:
R¹ represents C1-C20 alkyl, such as octanoyl or 4-methylhexanoyl,
R² represents

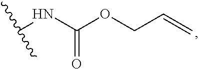 [SMILES: *NC(=O)OCC=C]

NH₂, OH,

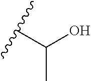 [SMILES: *C(C)O]

or H, and
X represents OH or NH₂.

According to an eleventh aspect, the present invention provides a compound of formula (VIII):

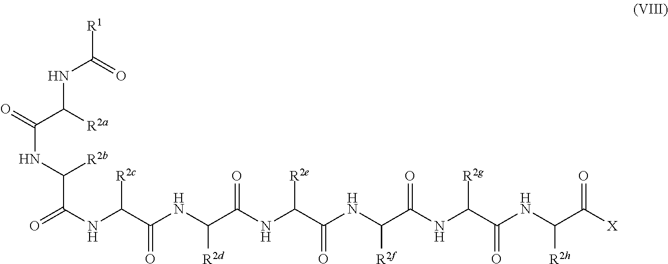

including tautomeric or stereochemically isomeric forms thereof, wherein:
R¹ represents C1-20 alkyl, C2-20 alkenyl, C6-20 aryl, or C4-20 heterocyclyl, each optionally substituted with one or more Y groups,
R^{2a} to R^{2h} each independently represents H, C1-10 alkyl, C2-10 alkenyl, C6-12 aryl, or C4-12 heterocyclyl, each optionally substituted with one or more Y groups,
R³ represents H, C1-10 alkyl, C2-10 alkenyl, C6-12 aryl, or C4-12 heterocyclyl, each optionally substituted with one or more Y groups,
X independently represents NH, S or O,
each Y independently represents cyano, halogen, N₃, —C(O)R^{Z}, —C(O)OR^{Z}, —OC(O)R^{Z}, —C(O)NHR^{Z}, —NHC(O)R^{Z}, —NHC(O)NHR^{Z}, —NHC(NH)NHR^{Z}, —NHC(O)OR^{Z}, —OC(O)NHR^{Z}, —OS(O)₂R^{Z}, —S(O)₂NHR^{z}, —NHS(O)₂R^{Z}, —SR^{Z}, —NR^{Z}₂ or —OR^{Z}, and
each R^{Z} independently represents H, C1-10 alkyl, C2-10 alkenyl, C6-10 aryl, or C4-10 heterocyclyl,
or an N-oxide thereof or a pharmaceutically acceptable salt thereof or a pharmaceutically acceptable solvate thereof.

Formula (VIII) may be represented by the following structure:

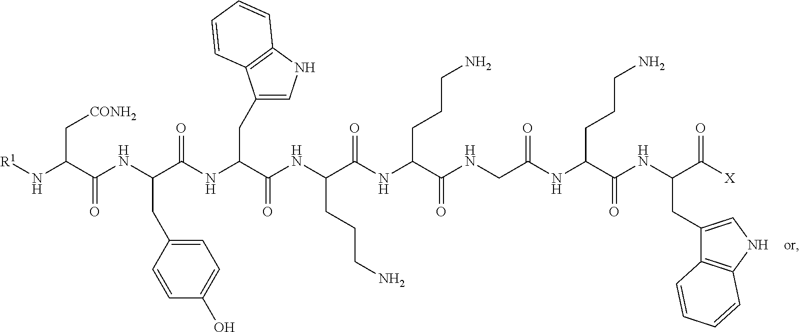

preferably:

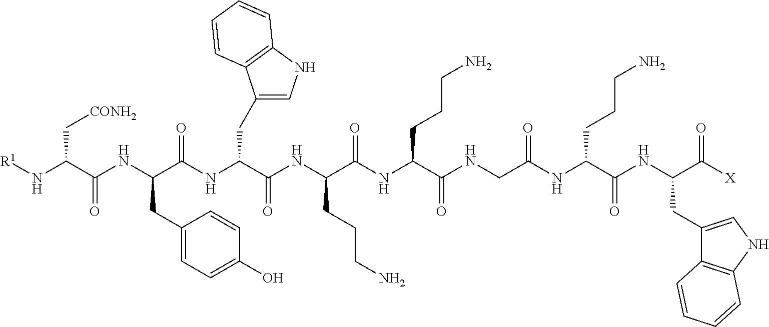

wherein:
R¹ represents C1-C20 alkyl, such as octanoyl or 4-methylhexanoyl, and
X represents OH or NH₂.

According to a twelfth aspect, the invention provides a pharmaceutical composition comprising the compound of the seventh, eighth, ninth, tenth or eleventh aspect, and a pharmaceutically acceptable carrier or diluent.

According to a thirteenth aspect, the invention provides a compound according to the seventh, eighth, ninth, tenth or eleventh aspect, or a pharmaceutical composition according to the twelfth aspect for use in therapy.

According to a fourteenth aspect, the invention provides a compound according to the seventh, eighth, ninth, tenth or eleventh aspect, or a pharmaceutical composition according to the twelfth aspect for use as an antibiotic. In particular, the use may be as an antibiotic against Gram-negative bacteria such as E. coli, K. pneumoniae, A. baumannii, and/or P. aeruginosa. The use may be in the treatment or prevention of bacterial infections, in particular those caused by Gram-negative bacteria such as E. coli, K. pneumoniae, A. baumannii, and/or P. aeruginosa, or strains thereof.

According to a fifteenth aspect, the invention provides a method of making a pharmaceutical composition according to the twelfth aspect, comprising the step of mixing a compound according to the seventh, eighth, ninth, tenth or eleventh aspect with a pharmaceutically acceptable carrier or diluent.

According to a sixteenth aspect, the invention provides a method of treating individuals suffering from bacterial infection, the method comprising administering an effective amount of a compound according to the seventh, eighth, ninth, tenth or eleventh aspect, or a pharmaceutical composition according to the twelfth aspect.

The optional functional group preferences described above in relation to Formulae (I) and/or (II) may apply equally with regard to any of Formulae (III) to (VIII), except where they are contradictory with the following statements relating to preferences for compounds of Formulae (III) to (VIII).

For formulae (III) to (VIII), R¹ may be represented by a 3-methylpentyl group, as found in brevicidine:

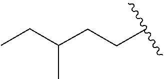 [SMILES: *CCC(C)CC]

For formulae (III) to (VIII), more preferably, R^{2a} is substituted with a Y group represented by —C(O)OH, —C(O)NH₂, —S(O)₂NH₂, —NHS(O)₂H. Most preferably, for formulae (III) to (VIII), R^{2a} is substituted with a Y group represented by —C(O)NH₂.

Most preferably, for formulae (III) to (VIII), R^{2a} represents the following group:

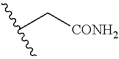 [SMILES: *CC(N)=O]

For Formulae (III), (IV) and/or (V), R^{2j} may represent H, C1-8 alkyl, C2-8 alkenyl, C6-8 aryl, or C4-8 heterocyclyl, each optionally substituted with one or more Y groups. For example, for Formulae (III), (IV) and/or (V), R^{2j} may represent H, C1-8 alkyl or C2-8 alkenyl each optionally substituted with one or more Y groups. Preferably, for Formulae (III), (IV) and/or (V), R^{2j} represent H, C1-8 alkyl or C2-8 alkenyl. For example, for Formulae (III), (IV) and/or (V), R^{2j} may represent H, C1-4 alkyl or C2-4 alkenyl, or H or C1-2 alkyl. Most preferably, for Formulae (III), (IV) and/or (V), R^{2j} represents H.

For formulae (III) and/or (IV), preferably, R^{2k} represents C1-6 alkyl or C2-6 alkenyl, such as C1-4 alkyl or C2-4 alkenyl, preferably C1 or C2 alkyl, each optionally substituted with one or more Y groups. For formulae (III) and/or (IV), preferably, R^{2k} k is substituted with one Y group represented by —NR^{Z}₂ or —OR^{Z}, more preferably —NH₂ or —OH, most preferably —OH. For formulae (III) and/or (IV), preferably, R^{2k} represents C1-6 alkyl or C2-6 alkenyl substituted with one Y group represented by —NR^{Z}₂ or —OR^{Z}, more preferably R^{2k} represents C1 or C2 alkyl substituted with one Y group represented by —NH₂ or —OH. Most preferably, for formulae (III) and/or (IV), R^{2k} represents the following group:

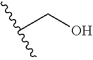 [SMILES: *CO]

For formulae (IV) to (VIII), XR^{Z} preferably represents XH, i.e. OH, NH₂ or SH. More preferably XR^{Z} represents NH₂ or OH. Most preferably XR^{Z} represents NH₂.

The compounds of the present invention are not limited to any particular stereoisomer. However, in one embodiment, the stereochemical configuration of the amino acid backbone of brevicidine and/or the isoleucine group R²ⁱ is used for compounds of Formulae (III) and/or (IV):

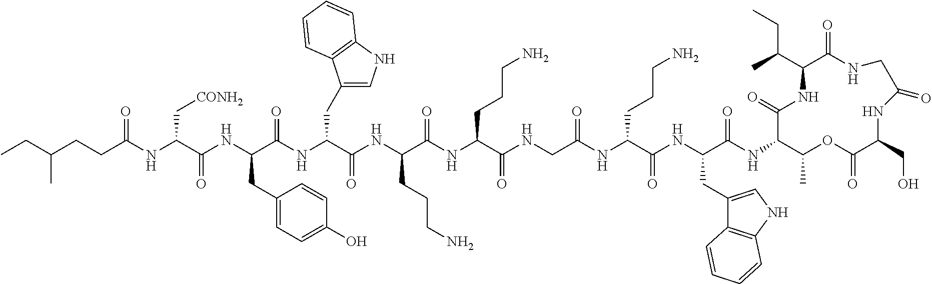 [SMILES: CCC(C)CCC(=O)N[C@H](CC(N)=O)C(=O)N[C@H](Cc1ccc(O)cc1)C(=O)N[C@H](Cc1c[nH]c2ccccc12)C(=O)N[C@H](CCCN)C(=O)N[C@@H](CCCN)C(=O)NCC(=O)N[C@H](CCCN)C(=O)N[C@@H](Cc1c[nH]c2ccccc12)C(=O)N[C@@H]1C(=O)N[C@@H]([C@@H](C)CC)C(=O)NCC(=O)N[C@@H](CO)C(=O)O[C@@H]1C]

In one embodiment the stereochemical configuration of the amino acid backbone and/or the isoleucine group R²ⁱ of the following structure is used for compounds of formula (V) to (VII):

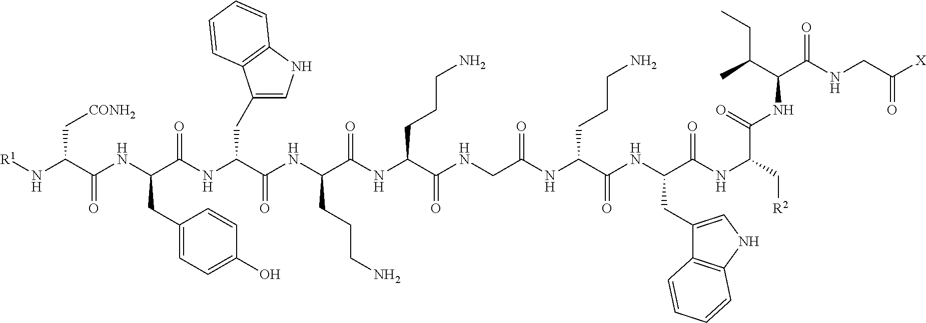

It may be the case that, for Formulae (III) to (VIII) specifically:
R¹ represents C6-12 alkyl,
R^{2a} represents C1-4 alkyl substituted with one Y group represented by —C(O)OH or —C(O)NH₂,
R^{2b} represents C6-10 aryl substituted with one Y group represented by —OH.
R^{2c} represents C4-12 heteroaryl,
R^{2d} represents C1-6 alkyl substituted with one Y group represented by —NH₂,
R^{2e} represents C1-6 alkyl substituted with one Y group represented by —NH₂,
R^{2f} represents H or C1-2 alkyl,
R^{2g} represents C1-6 alkyl substituted with one Y group represented by —NH₂,
R^{2h} represents C4-12 heteroaryl,
R²ⁱ represents branched C3-5 alkyl or branched C3-5 alkenyl,
R^{2j} represents H or C1-2 alkyl,
Any R^{2k} represents C1-4 alkyl substituted with one Y group represented by —OH,
R³ represents H, C1-4 alkyl, C2-4 alkenyl, optionally substituted with one Y group represented by N₃, —C(O)OR^{Z}, —OC(O)R^{Z}, —C(O)NHR^{Z}, —NHC(O)R^{Z}, —NHC(O)NHR^{Z}, —NHC(O)OR^{Z}, OC(O)NHR^{Z}, —NR^{Z}₂ or —OR^{z}, or optionally represented by —NHC(NH)NHR^{Z} or SR^{Z},
Each R^{Z} independently represents H, C1-4 alkyl or C2-4 alkenyl, and
X represents O or NH, preferably NH.

### Pharmaceutical Compositions

In one embodiment the pharmaceutical composition (e.g. formulation) comprises at least one active compound of the invention together with one or more pharmaceutically acceptable carriers, adjuvants, excipients, diluents, fillers, buffers, stabilisers, preservatives, lubricants, or other materials well known to those skilled in the art and optionally other therapeutic or prophylactic agents.

To prepare the pharmaceutical compositions of this invention, an effective amount of a compound of the present invention, as the active ingredient is combined in intimate admixture with a pharmaceutically acceptable carrier, which carrier may take a wide variety of forms depending on the form of preparation desired for administration. The pharmaceutical compositions can be in any form suitable for oral, parenteral, topical, intranasal, ophthalmic, otic, rectal, intra-vaginal, intravenous or transdermal administration.

It may be advantageous to formulate the aforementioned pharmaceutical compositions in dosage unit form for ease of administration and uniformity of dosage. Dosage unit form as used in the specification and claims herein refers to physically discrete units suitable as unitary dosages, each unit containing a predetermined quantity of active ingredient, calculated to produce the desired therapeutic effect, in association with the required pharmaceutical carrier. Examples of such dosage unit forms are tablets (including scored or coated tablets), capsules, pills, powder packets, wafers, injectable solutions or suspensions, teaspoonfuls, tablespoonfuls and the like, and segregated multiples thereof.

The compound of the invention is to be administered in an amount sufficient to exert its antibiotic activity.

The compounds according to the invention may be administered to a human or to an animal, which may be a mammal, e.g. it may be a farm animal or an animal kept as a pet, such as a dog, cat or horse, or a laboratory animal, such as a rat, rabbit, guinea pig or dog.

The present invention further relates to the use of a compound according to the invention in the manufacture of a pharmaceutical composition. The pharmaceutical composition may, for example, be for use an antibiotic, i.e. for inhibiting the growth of bacteria.

### Examples

### 1. Synthetic Challenges

Initial efforts to synthesise compounds such as laterocidine, relacidine and brevicidine were unsuccessful.

However, the present inventors have provided a synthetic route to these compounds and related analogues, allowing humankind to access a broad range of new and useful analogues of laterocidine, relacidine and brevicidine.

Initially approaching the synthesis of brevicidine, the inventors initially investigated a strategy starting from Gly 1 loaded on 2-chlorotrityl chloride (CTC) to generate a linear peptide that would subsequently be cyclized in solution. To do so the required Thr9-Ser12 linkage was envisioned to be installed as a preformed, ester-linked dipeptide.

However, while incorporation of the Thr9-Ser12 unit was achieved, further elongation of the peptide failed due to an O→N acyl shift that occurred upon removal of the Thr9 Fmoc group.

Therefore, synthesis of brevicidine was not successful, or facile. These synthetic problems would also apply to its analogues.

In the synthesis of laterocidine, the successful route to synthesise brevicidine, described in the following sections, was followed and modified as was thought to be necessary. However, this was not successful for laterocidine.

To begin, Fmoc-Asp-OAll was loaded onto Rink amide resin via its free side chain carboxylate. Following allyl ester removal, an allyl ester protected Gly-Gly dipeptide was next coupled. The peptide was then built out to the Trp8 residue (so as to avoid possible O→N acyl migration later on in the synthesis) with Thr9 successfully installed without side chain protection.

Following allyl ester deprotection, formation of the macrolactone was attempted using a variety of conditions, including the modified Yamaguchi esterification. However, frustratingly, all conditions attempted failed to provide the desired product.

Therefore, synthesis of laterocidine (and, by virtue of its related structure, relacidine) was not facile, These synthetic problems would also apply to its analogues.

### 2. Successful Synthesis of Compounds

It should be understood that the following syntheses could be readily adopted and modified by the skilled person in order to synthesise other compounds of the present invention.

Starting materials were either commercially available, from sources such as Sigma-Aldrich®, or synthesised from commercially available materials using methods and techniques well-known to the skilled person. Fmoc-Ser-OAll (S. Mukherjee, W. A. van der Donk. J. Am. Chem. Soc. 2014, 136, 10450-10459), Fmoc-Asp-OAll (J. Ge, L. Li, S. Q. Yao, Chem. Commun. 2011, 47, 10939-10941), TFA NH₂-Gly-OAll (F. Freire, J. D. Fisk, A. J. Peoples, M. Ivancic, I. A. Guzei, S. H. Gellman, J. Am. Chem. Soc. 2008, 130, 7839-7841), TFA NH₂-Gly-Gly-OAll (C. Chun, S. M. Lee, S. Y. Kim, H. K. Yang, S. C. Song, Biomaterials 2009, 30, 2349-2360), and (2S,3R)-2-((((9H-fluoren-9-yl)methoxy)carbonyl)amino)-3-azidobutanoic acid (R. Moreira, G. Barnawi, D. Beriashvili, M. Palmer, S. D. Taylor, Biorg. Med. Chem. 2019, 27, 240-246) were synthesized according to the referenced literature procedures.

Brevicidine and analogues were purified using a Perkin Elmer HPLC system composed of a 200 series binary pump, UV/Vis detector monitoring at 220 nm, vacuum degasser and Rheodyne 7725i injector. Method A: Phenomenex Luna C18 column (21.2×250 mm, 5 μm) with a 2 mL injection loop. The following solvent system, at a flow rate of 10 mL/min, was used: solvent A, 0.1% TFA in water; solvent B, acetonitrile. Gradient elution was as follows: 80:20 (A/B) for 5 min, 80:20 to 45:55 (A/B) over 30 min, 45:55 to 0:95 (A/B) over 3 min, 0:95 (A/B) for 3 min then reversion back to 80:20 (A/B) over 2 min, 80:20 (A/B) for 5 min. Method B: Phenomenex C18 Luna column (4.6×150 mm, 5 μm) with a 200 μL injection loop. The following solvent system, at a flow rate of 2 mL/min, was used: solvent A, 0.1% TFA in water; solvent B, acetonitrile. Gradient elution was as follows: 95:5 (A/B) for 2 min, 95:5 to 5:95 (A/B) over 18 min then reversion back to 95:5 (A/B) over 0.1 min, 95:5 (A/B) for 3.9 min. Laterocidine and analogues were purified using the following methods. Method C: BESTA-Technik system equipped with a ECOM Flash UV detector monitoring at 214 nm and 254 nm with a Dr. Maisch Reprosil Gold 120 C18 column (25×250 mm, 10 μm). The following solvent system, at a flow rate of 12 mL/min, was used: solvent A, 0.1% TFA in water/acetonitrile 95/5; solvent B, 0.1% TFA in water/acetonitrile 5/95. Gradient elution was as follows: 100:0 (A/B) for 5 min, 100:0 to 50:50 (A/B) over 50 min, 50:50 to 0:100 (A/B) for 3 min, then reversion back to 100:0 (A/B) over 1 min, 100:0 (A/B) for 5 min. Method D: Shimadzu Prominence-i LC-2030 system with a Dr. Maisch ReproSil Gold 120 C18 column (4.6×250 mm, 5 μm) at 30° C. and equipped with a UV detector monitoring 214 nm and 254 nm. The following solvent system, at a flow rate of 1 mL/min, was used: solvent A, 0.1% TFA in water/acetonitrile 95/5; solvent B, 0.1% TFA in water/acetonitrile 5/95. Gradient elution was as follows: 100:0 (A/B) for 2 min, 100:0 to 50:50 (A/B) over 45 min, 50:50 (A/B) to 0:100 (A/B) over 1 min, 0:100 (A/B) for 6 min then reversion back to 100:0 (A/B) over 1 min, 100:0 (A/B) for 5 min.

### 2.1. Laterocidine-Related Compounds

### 2.1.1. Laterocidine (2)

Rink Amide MBHA resin (2.0 g, 0.67 mmol g⁻¹) was loaded by overnight coupling via the free sidechain carboxylate of Fmoc-Asp-OAll (2.65 g, 6.70 mmol, 2 eq.) with BOP (2.96 g, 6.70 mmol, 2 eq.) and DiPEA (2.33 mL, 13.4 mmol, 4 eq.) in 150 mL of DMF. After capping with Ac₂O:pyridine (3:2, v/v) for 30 min the resin loading was determined to be 0.37 mmol g⁻¹. The loaded resin (680 mg, 0.25 mmol) was treated with Pd(PPh₃)₄ (75 mg, 0.075 mmol, 0.3 eq.) and PhSiH₃ (0.75 mL, 7.5 mmol, 30 eq.) in DCM (ca. 15 mL) under nitrogen for 1 hour. The resin was subsequently washed with DCM (5×10 mL), followed by a solution of diethyldithiocarbamic acid trihydrate sodium salt (5 mg mL⁻¹ in DMF, 5×10 mL), and DMF (5×10 mL). TFA H₂N-Gly-OAll (115 mg, 0.5 mmol, 2 eq.) was then coupled using BOP (221 mg, 0.5 mmol, 2 eq.) and DiPEA (174 μL, 1.0 mmol, 4 eq.) under nitrogen flow for 1 hour. The next 3 amino acids (Ile10, Thr9, Trp8) were coupled manually (1 h) under nitrogen flow via standard Fmoc solid-phase peptide synthesis (SPPS) (resin:Fmoc-AA:BOP:DiPEA, 1:4:4:8 molar eq.). DMF (5 mL) was used as solvent and Fmoc deprotections (2 min then 10 min) were carried out with 5 mL piperidine:DMF (1:4, v/v). The following Fmoc amino acids were used: Fmoc-Ile-OH, Fmoc-Thr-OH (used without side chain protection), and Fmoc-Trp(Boc)-OH. After coupling of Fmoc-Trp(Boc)-OH esterification of the Thr side chain was achieved by treating the resin-bound peptide with Alloc-Gly-OH (596 mg, 3.75 mmol, 15 eq.), DIC (0.59 mL, 3.75 mmol, 15 eq.) and DMAP (15 mg, 0.13 mmol, 0.5 eq.) in 8 mL DCM:DMF (3:1, v/v) for 18 h under nitrogen. The resin was treated with Pd(PPh₃)₄ (75 mg, 0.075 mmol) and PhSiH₃ (0.75 mL, 7.5 mmol) in DCM (ca. 15 mL) under nitrogen for 2 h before being washed with DCM (5×10 mL), followed by a solution of diethyldithiocarbamic acid trihydrate sodium salt (5 mg mL⁻¹ in DMF, 5×10 mL), and DMF (5×10 mL). The peptide was then cyclized using BOP (442 mg, 1.0 mmol, 4 eq.) and DiPEA (0.35 mL, 2.0 mmol, 8 eq.) for 2 h in 5 mL DMF under nitrogen flow. The remaining linear N-terminal section of the peptide was then synthesized using the standard SPPS protocol mentioned above. The following Fmoc amino acids were used: Fmoc-D-Ser(tBu)-OH, Fmoc-D-Tyr(tBu)-OH, Fmoc-D-Trp(Boc)-OH, Fmoc-L-Orn(Boc)-OH, Fmoc-L-Orn(Boc)-OH, Fmoc-Gly. Following the final Fmoc removal step, isopelargonic acid (79 mg, 0.5 mmol, 2 eq.) was coupled using BOP (221 mg, 0.5 mmol, 2 eq.) and DiPEA (174 μL, 1.0 mmol, 4 eq.) in 5 mL of DMF overnight, under nitrogen flow. Final deprotection was carried out by treating the resin with TFA:TIS:H₂O (95:2.5:2.5, 10 mL) for 90 min. The reaction mixture was filtered through cotton, the filtrate precipitated in MTBE:petroleum ether (1:1), and the resulting precipitate washed once more with MTBE:petroleum ether (1:1). The crude cyclic peptide was lyophilized from tBuOH:H₂O (1:1) and purified with reverse phase HPLC. Pure fractions were pooled and lyophilized to yield laterocidine in >95% purity as a white powder. Comparison of analysis of the synthesised compound with laterocidine isolated from natural sources provided an exact match.

### 2.1.2. Synthesis of the Enantiomer of Laterocidine (Ent-Laterocidine, Ent-2)

Rink Amide MBHA resin (2.0 g, 0.67 mmol g⁻¹) was loaded by overnight coupling via the free sidechain carboxylate of Fmoc-D-Asp-OAll (2.65 g, 6.70 mmol, 2 eq.) with BOP (2.96 g, 6.70 mmol, 2 eq.) and DiPEA (2.33 mL, 13.4 mmol, 4 eq.) in 150 mL of DMF. After capping with AcO2:pyridine (3:2, v/v) for 30 min the resin loading was determined to be 0.37 mmol g⁻¹. The loaded resin (675 mg, 0.25 mmol) was treated with Pd(PPh₃)₄ (75 mg, 0.075 mmol, 0.3 eq.) and PhSiH₃ (0.75 mL, 7.5 mmol, 30 eq.) in DCM (ca. 15 mL) under nitrogen for 1 h. The resin was subsequently washed with DCM (5×10 mL), followed by a solution of diethyldithiocarbamic acid trihydrate sodium salt (5 mg mL⁻¹ in DMF, 5×10 mL), and DMF (5×10 mL). TFA H₂N-Gly-OAll (115 mg, 0.5 mmol, 2 eq.) was then coupled using BOP (221 mg, 0.5 mmol, 2 eq.) and DiPEA (174 μL, 1.0 mmol, 4 eq.) under nitrogen flow for 1 h. The next 3 amino acids (D-Ile10, D-Thr9, D-Trp8) were coupled manually (1 h) under nitrogen flow via standard Fmoc solid-phase peptide synthesis (SPPS) (resin:Fmoc-AA:BOP:DiPEA, 1:4:4:8 molar eq.). DMF (5 mL) was used as solvent and Fmoc deprotections (2 min then 10 min) were carried out with 5 mL piperidine:DMF (1:4, v/v). The following Fmoc amino acids were used: Fmoc-D-Ile-OH, Fmoc-D-Thr-OH (used without side chain protection), and Fmoc-D-Trp(Boc)-OH. After coupling of Fmoc-D-Trp(Boc)-OH esterification of the D-Thr side chain was achieved by treating the resin-bound peptide with Alloc-Gly-OH (596 mg, 3.75 mmol, 15 eq.), DIC (0.59 mL, 3.75 mmol, 15 eq.) and DMAP (15 mg, 0.13 mmol, 0.5 eq.) in 8 mL DCM:DMF (3:1, v/v) for 18 h under nitrogen. The resin was treated with Pd(PPh₃)₄ (75 mg, 0.075 mmol) and PhSiH₃ (0.75 mL, 7.5 mmol) in DCM (ca. 15 mL) under nitrogen for 2 h before being washed with DCM (5×10 mL), followed by a solution of diethyldithiocarbamic acid trihydrate sodium salt (5 mg mL⁻¹ in DMF, 5×10 mL), and DMF (5×10 mL). The peptide was then cyclized using BOP (442 mg, 1.0 mmol, 4 eq.) and DiPEA (0.35 mL, 2.0 mmol, 8 eq.) for 2 h in 5 mL DMF under nitrogen flow. The remaining linear N-terminal section of the peptide was then synthesized using the standard SPPS protocol mentioned above. The following Fmoc amino acids were used: Fmoc-L-Ser(tBu)-OH, Fmoc-L-Tyr(tBu)-OH, Fmoc-L-Trp(Boc)-OH, Fmoc-L-Orn(Boc)-OH, Fmoc-D-Orn(Boc)-OH, Fmoc-Gly-OH. Following the final Fmoc removal step, isopelargonic acid (79 mg, 0.5 mmol, 2 eq.) was coupled using BOP (221 mg, 0.5 mmol, 2 eq.) and DiPEA (174 μL, 1.0 mmol, 4 eq.) in 5 mL of DMF overnight, under nitrogen flow. Final deprotection was carried out by treating the resin with TFA:TIS:H2O (95:2.5:2.5, 10 mL) for 90 min. The reaction mixture was filtered through cotton, the filtrate precipitated in MTBE:petroleum ether (1:1), and the resulting precipitate washed once more with MTBE:petroleum ether (1:1). The crude cyclic peptide was lyophilized from tBuOH:H₂O (1:1) and purified with reverse phase HPLC (See HPLC purification of synthetic peptides). Pure fractions were pooled and lyophilized to yield ent-laterocidine in >95% purity as a white powder. Yield: 30 mg, 7.4% over 29 steps. [M+2H]²⁺ calculated for, C₇₈H₁₁₃NgO₁₈ 802,9329, found (HR-MS) 802,9327.

### 2.1.3. Stereochemically Altered Laterocidine Analogues

Stereochemically altered analogues of laterocidine, each having inverted stereochemistry at a different amino acid residue compared to the naturally occurring stereochemistry, was performed using similar techniques to those to synthesise ent-laterocidine and laterocidamide.

### 2.1.4. Laterocidamide (Dap9-Laterocidine, 6, Solution Phase Cyclization Route)—Formula (I)

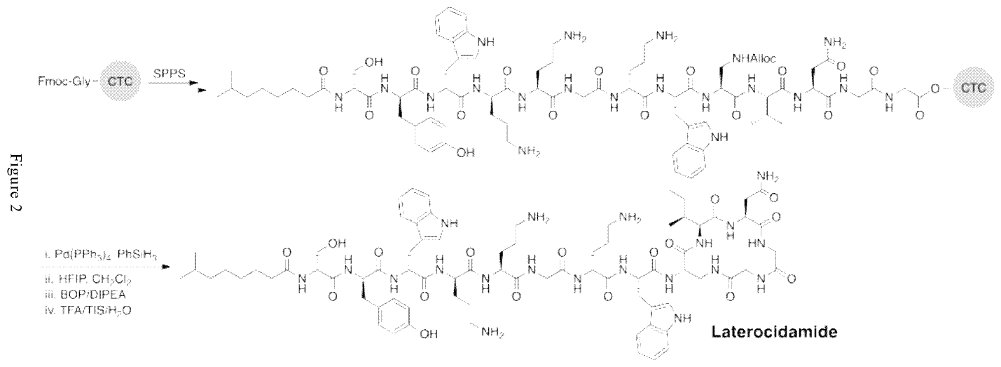

FIG. 2 depicts the solution phase cyclisation synthesis of laterocidamide. 2-Chlorotrityl resin (5.0 g, 1.60 mmol g⁻¹) was loaded with Fmoc-Gly-OH. Resin loading was determined to be 0.67 mmol g⁻¹. The linear peptide was assembled manually on a 0.25 mmol scale under nitrogen flow via standard Fmoc solid-phase peptide synthesis (SPPS) (1 h couplings, resin:Fmoc-AA:BOP:DiPEA, 1:4:4:8 molar eq.). DMF (5 mL) was used as solvent and Fmoc deprotections (2 min then 10 min) were carried out with 5 mL piperidine:DMF (1:4, v/v). The following Fmoc amino acids were used: Fmoc-D-Ser(tBu)-OH, Fmoc-D-Tyr(tBu)-OH, Fmoc-D-Trp(Boc)-OH, Fmoc-D-Orn(Boc)-OH, Fmoc-L-Orn(Boc)-OH, Fmoc-Gly-OH, Fmoc-L-Trp(Boc)-OH, Fmoc-L-Dap(Alloc)-OH, Fmoc-Ile-OH, and Fmoc-Asn(Trt)-OH (where consecutive amino acids are identical, repetitions have been omitted from this list, but the skilled person will understand that the sequence follows the structure of laterocidamide). Following the final Fmoc removal step, isopelargonic acid (79 mg, 0.5 mmol, 2 eq.) was coupled using BOP (221 mg, 0.5 mmol, 2 eq.) and DiPEA (174 μL, 1.0 mmol, 4 eq.) in 5 mL of DMF overnight, under nitrogen flow. The resin was then treated two times with Pd(PPh₃)₄ (75 mg, 0.075 mmol) and PhSiH₃ (0.75 mL, 7.5 mmol) in CH₂Cl₂ (ca. 15 mL) under nitrogen for 3 hours with washing in between with CH₂Cl₂ (5×10 mL), followed by a solution of diethyldithiocarbamic acid trihydrate sodium salt (5 mg mL⁻¹ in DMF, 5×10 mL), and DMF (5×10 mL). The peptide was cleaved off the resin by treating it with HFIP:DCM (1:4, v/v, 20 mL) for 1 hour and rinsed with additional HFIP:DCM and DCM. The combined washings were then evaporated to yield the linear protected peptide with a free C-terminus and Dap amino sidechain. The partially protected peptide was dissolved in DCM (150 mL), treated with BOP (0.22 g, 0.5 mmol) and DiPEA (0.17 mL, 1.0 mmol) and the solution was stirred overnight under nitrogen atmosphere. The reaction mixture was concentrated and directly treated with TFA:TIS:H₂O (95:2.5:2.5, 10 mL) for 90 min. The reaction mixture was subsequently filtered through cotton, the filtrate was precipitated in MTBE:petroleum ether (1:1) and the resulting precipitate washed once more with MTBE:petroleum ether (1:1). The crude cyclic peptide was lyophilized from tBuOH:H₂O (1:1) and purified with reverse phase HPLC. Pure fractions were pooled and lyophilized to yield laterocidamide in >95% purity as a white powder. Yield: 39 mg, 10% over 30 steps.

2.1.5. Laterocidamide (On-Resin Cyclization Route)—Formula (I)

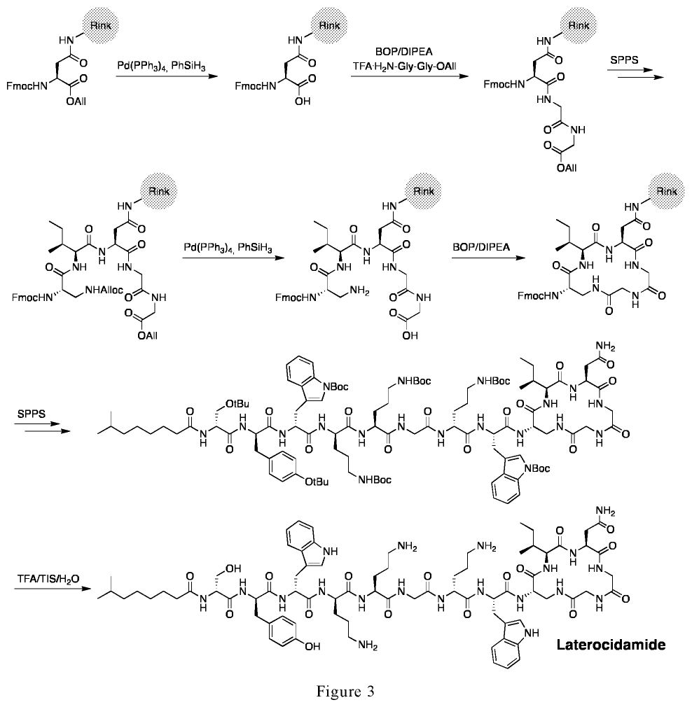

FIG. 3 depicts the on-resin cyclisation synthesis of laterocidamide.

Rink Amide MBHA resin (2.0 g, 0.67 mmol g⁻¹) was loaded by overnight coupling via the free sidechain carboxylate of Fmoc-Asp-OAll (2.65 g, 6.70 mmol, 2 eq.) with BOP (2.96 g, 6.70 mmol, 2 eq.) and DiPEA (2.33 mL, 13.4 mmol, 4 eq.) in 150 mL of DMF. After capping with AcO₂:pyridine (3:2, v/v) for 30 min the resin loading was determined to be 0.37 mmol g⁻¹. The loaded resin (680 mg, 0.25 mmol) was treated with Pd(PPh₃)₄ (75 mg, 0.075 mmol, 0.3 eq.) and PhSiH₃ (0.75 mL, 7.5 mmol, 30 eq.) in DCM (ca. 15 mL) under nitrogen for 1 hour. The resin was subsequently washed with CH₂Cl₂ (5×10 mL), followed by a solution of diethyldithiocarbamic acid trihydrate sodium salt (5 mg mL⁻¹ in DMF, 5×10 mL), and DMF (5×10 mL). TFA H₂N-Gly-Gly-OAll (143 mg, 0.5 mmol, 2 eq.) was then coupled using BOP (221 mg, 0.5 mmol, 2 eq.) and DiPEA (174 μL, 1.0 mmol, 4 eq.) under nitrogen flow for 1 hour. The next two amino acids (Ile and Dap) were coupled manually (1 h) under nitrogen flow via standard Fmoc solid-phase peptide synthesis (SPPS) (resin:Fmoc-AA:BOP:DiPEA, 1:4:4:8 molar eq.). DMF (5 mL) was used as solvent and Fmoc deprotections (2 min then 10 min) were carried out with 5 mL piperidine:DMF (1:4, v/v). The following Fmoc amino acids were used: Fmoc-Ile-OH and Fmoc-L-Dap(Alloc)-OH. The resin was then treated two times with Pd(PPh₃)₄ (75 mg, 0.075 mmol) and PhSiH₃ (0.75 mL, 7.5 mmol) in DCM (ca. 15 mL) under nitrogen for 3 hours with washing in between with DCM (5×10 mL), followed by a solution of diethyldithiocarbamic acid trihydrate sodium salt (5 mg mL⁻¹ in DMF, 5×10 mL), and DMF (5×10 mL). The macrocycle was then closed by treatment with BOP (442 mg, 1.0 mmol, 4 eq.) and DiPEA (0.35 mL, 2.0 mmol, 8 eq.) for 2 h in 5 mL DMF under nitrogen flow. Following cyclization the remaining linear N-terminal section of the peptide was added using a CEM Liberty Blue automated peptide synthesizer with microwave irradiation, on standard settings (resin:Fmoc-AA:DIC:Oxyma, 1:5:5:5 molar eq.). DMF was used as solvent and Fmoc deprotections were carried out with piperidine:DMF (1:4, v/v). The following Fmoc amino acids were used: Fmoc-D-Ser(tBu)-OH, Fmoc-D-Tyr(tBu)-OH, Fmoc-D-Trp(Boc)-OH, Fmoc-D-Orn(Boc)-OH, Fmoc-L-Orn(Boc)-OH, Fmoc-Gly-OH, Fmoc-L-Trp(Boc)-OH. Following the final Fmoc removal step, the resin was removed from the CEM Liberty Blue and washed with DCM and DMF before isopelargonic acid (79 mg, 0.5 mmol, 2 eq.) was coupled manually using BOP (221 mg, 0.5 mmol, 2 eq.) and DiPEA (174 μL, 1.0 mmol, 4 eq.) in 5 mL of DMF overnight, under nitrogen flow. Final deprotection was carried out by treating the resin with TFA:TIS:H₂O (95:2.5:2.5, 10 mL) for 90 min. The reaction mixture was filtered through cotton, the filtrate was precipitated in MTBE:petroleum ether (1:1) and the resulting precipitate washed once more with MTBE:petroleum ether (1:1). The crude cyclic peptide was lyophilized from tBuOH:H₂O (1:1) and purified with reverse phase HPLC. Pure fractions were pooled and lyophilized to yield laterocidamide in >95% purity as a white powder. Yield: 10 mg, 3% over 27 steps.

### 2.1.1. Synthesis of Ser9-Laterocidine (4)

The loaded resin (274 mg, 0.1 mmol) was treated with Pd(PPh₃)₄ (30 mg, 0.03 mmol, 0.3 eq.) and PhSiH₃ (0.30 mL, 3.0 mmol, 30 eq.) in DCM (ca. 7 mL) under nitrogen for 1 h. The resin was subsequently washed with DCM (5×10 mL), followed by a solution of diethyldithiocarbamic acid trihydrate sodium salt (5 mg mL⁻¹ in DMF, 5×10 mL), and DMF (5×10 mL). TFA·H₂N-Gly-OAll (115 mg, 0.5 mmol, 2 eq.) was then coupled using BOP (88 mg, 0.2 mmol, 2 eq.) and DiPEA (87 μL, 0.4 mmol, 4 eq.) under nitrogen flow for 2 h. The next 3 amino acids (Ile10, Ser9, Trp8) were coupled manually (1 h) under nitrogen flow via standard Fmoc solid-phase peptide synthesis (SPPS) (resin:Fmoc-AA:BOP:DiPEA, 1:4:4:8 molar eq.). Dry DMF (3 mL) was used as solvent and Fmoc deprotections (2 min then 10 min) were carried out with 3 mL piperidine:DMF (1:4, v/v). The following Fmoc amino acids were used: Fmoc-Ile-OH, Fmoc-Ser-OH (used without side chain protection), and Fmoc-Trp(Boc)-OH. After coupling of Fmoc-Trp(Boc)-OH esterification of the Ser side chain was achieved by treating the resin-bound peptide with Alloc-Gly-OH (238 mg, 1.5 mmol, 15 eq.), DIC (0.24 mL, 1.5 mmol, 15 eq.) and DMAP (6 mg, 0.05 mmol, 0.5 eq.) in 3 mL DCM:DMF (3:1, v/v) for 18 h under nitrogen. The resin was treated with PhSiH₃ (0.30 mL, 3.0 mmol, 30 eq.) in DCM (ca. 7 mL) under nitrogen for 2 h before being washed with DCM (5×10 mL), followed by a solution of diethyldithiocarbamic acid trihydrate sodium salt (5 mg mL⁻¹ in DMF, 5×10 mL), and DMF (5×10 mL). The peptide was then cyclized using BOP (177 mg, 0.4 mmol, 4 eq.) and DiPEA (0.14 mL, 0.8 mmol, 8 eq.) for 2 h in 3 mL DMF under nitrogen flow. The remaining linear N-terminal section of the peptide was then synthesized using the standard SPPS protocol mentioned above. The following Fmoc amino acids were used: Fmoc-D-Ser(tBu)-OH, Fmoc-D-Tyr(tBu)-OH, Fmoc-D-Trp(Boc)-OH, Fmoc-D-Orn(Boc)-OH, Fmoc-L-Orn(Boc)-OH, Fmoc-Gly. Following the final Fmoc removal step, isopelargonic acid (79 mg, 0.5 mmol, 2 eq.) was coupled using BOP (88 mg, 0.2 mmol, 2 eq.) and DiPEA (87 μL, 0.4 mmol, 4 eq.) in 3 mL of DMF overnight, under nitrogen flow. Final deprotection was carried out by treating the resin with TFA:TIS:H2O (95:2.5:2.5, 5 mL) for 90 min. The reaction mixture was filtered through cotton, the filtrate precipitated in MTBE:petroleum ether (1:1), and the resulting precipitate washed once more with MTBE:petroleum ether (1:1). The crude cyclic peptide was lyophilized from tBuOH:H₂O (1:1) and purified with reverse phase HPLC (See HPLC purification of synthetic peptides). Pure fractions were pooled and lyophilized to yield laterocidine in >95% purity as a white powder. Yield: 4 mg, 2% over 29 steps [M+2H]²⁺ calculated for, C₇₈H₁₁₃N₁₉O₁₈ 795.9250, found (HR-MS) 795,9249.

### 2.1.2. Synthesis of MeDap9-Laterocidine (8)

Rink amide MBHA resin loaded with Fmoc-Asp-OAll (680 mg, 0.25 mmol) was treated with Pd(PPh₃)₄ (75 mg, 0.075 mmol) and PhSiH₃ (0.75 mL, 7.5 mmol) in DCM (ca. 15 mL) under nitrogen for 2 h before being washed with DCM (5×10 mL), followed by a solution of diethyldithiocarbamic acid trihydrate sodium salt (5 mg mL⁻¹ in DMF, 5×10 mL), and DMF (5×10 mL). TFA H₂N-Gly-OAll (115 mg, 0.5 mmol, 2 eq.) was then coupled using BOP (221 mg, 0.5 mmol, 2 eq.) and DiPEA (174 μL, 1.0 mmol, 4 eq.) under nitrogen flow for 1 h. The next 2 amino acids Fmoc-Ile-OH and (2S,3R)-Fmoc-azido-aminobutyric acid were coupled manually (1 h) under nitrogen flow via standard Fmoc solid-phase peptide synthesis (SPPS) (resin:Fmoc-AA:BOP:DiPEA, 1:4:4:8 molar eq.). DMF (5 mL) was used as solvent and Fmoc deprotections (2 min then 10 min) were carried out with 5 mL piperidine:DMF (1:4, v/v). The azide was then reduced using a DTT (2M) and DiPEA (1M) in DMF (ca. 15 mL) for 2H under N₂ followed by washings with DMF (5×10 mL). The Allyl protecting group on the C-terminus was then removed using Pd(PPh₃)₄ (75 mg, 0.075 mmol) and PhSiH₃ (0.75 mL, 7.5 mmol) in DCM (ca. 15 mL) under nitrogen for 2 h before being washed with DCM (5×10 mL), followed by a solution of diethyldithiocarbamic acid trihydrate sodium salt (5 mg mL⁻¹ in DMF, 5×10 mL), and DMF (5×10 mL). The peptide was then cyclized using BOP (442 mg, 1.0 mmol, 4 eq.) and DiPEA (0.35 mL, 2.0 mmol, 8 eq.) for 2 h in 5 mL DMF under nitrogen flow. The remaining linear N-terminal section of the peptide was then synthesized using the standard SPPS protocol mentioned above. The following Fmoc amino acids were used: Fmoc-D-Ser(tBu)-OH, Fmoc-D-Tyr(tBu)-OH, Fmoc-D-Trp(Boc)-OH, Fmoc-D-Orn(Boc)-OH, Fmoc-L-Orn(Boc)-OH, Fmoc-Gly. Fmoc-L-Trp(Boc)-OH. Following the final Fmoc removal step, isopelargonic acid (79 mg, 0.5 mmol, 2 eq.) was coupled using BOP (221 mg, 0.5 mmol, 2 eq.) and DiPEA (174 μL, 1.0 mmol, 4 eq.) in 5 mL of DMF overnight, under nitrogen flow. Final deprotection was carried out by treating the resin with TFA:TIS:H2O (95:2.5:2.5, 10 mL) for 90 min. The reaction mixture was filtered through cotton, the filtrate precipitated in MTBE:petroleum ether (1:1), and the resulting precipitate washed once more with MTBE:petroleum ether (1:1). The crude cyclic peptide was lyophilized from tBuOH:H₂O (1:1) and purified with reverse phase HPLC (See HPLC purification of synthetic peptides). Pure fractions were pooled and lyophilized to yield MeDap9-laterocidine in (9.7 mg, 2.4% over 29 steps, purity >95%) [M+2H]²⁺ calculated for, C₇₈H₁₁₄N₂₀O₁₇ 802.4408, found (HRMS) 802.4412.

### 2.1.3. Laterocidine Lipid Analogues

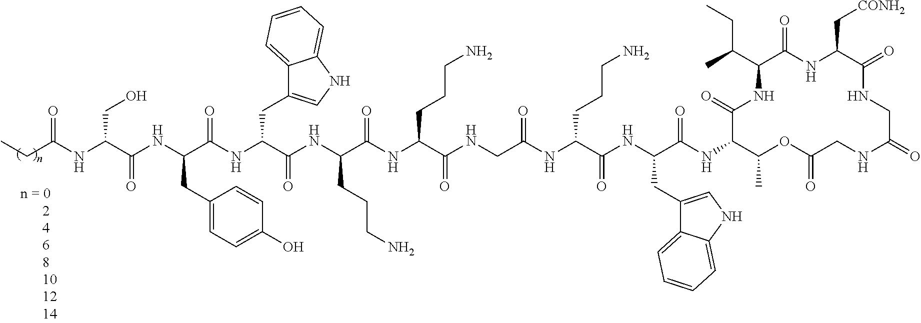

Rink Amide MBHA resin (5.0 g, 0.67 mmol g⁻¹) was loaded by overnight coupling via the free sidechain carboxylate of Fmoc-Asp-OAll (2.65 g, 6.70 mmol, 2 eq.) with BOP (2.96 g, 6.70 mmol, 2 eq.) and DiPEA (2.33 mL, 13.4 mmol, 4 eq.) in 150 mL of DMF. After capping with Ac₂O:pyridine (3:2, v/v) for 30 min the resin loading was determined to be 0.50 mmol g⁻¹. Two batches of the loaded resin (1.0 g, 0.5 mmol) were treated with Pd(PPh₃)₄ (150 mg, 0.15 mmol, 0.3 eq.) and PhSiH₃ (1.5 mL, 15 mmol, 30 eq.) in DCM (ca. 30 mL) under nitrogen for 1 hour. The resin was subsequently washed with DCM (5×20 mL), followed by a solution of diethyldithiocarbamic acid trihydrate sodium salt (5 mg mL⁻¹ in DMF, 5×20 mL), and DMF (5×20 mL). TFA H₂N-Gly-OAll (230 mg, 1.0 mmol, 2 eq.) was then coupled using BOP (442 mg, 1.0 mmol, 2 eq.) and DiPEA (0.35 mL, 2.0 mmol, 4 eq.) under nitrogen flow for 1 hour. The next 3 amino acids (Ile10, Thr9, Trp8) were coupled manually (1 h) under nitrogen flow via standard Fmoc solid-phase peptide synthesis (SPPS) (resin:Fmoc-AA:BOP:DiPEA, 1:4:4:8 molar eq.). DMF (10 mL) was used as solvent and Fmoc deprotections (2 min then 10 min) were carried out with 10 mL piperidine:DMF (1:4, v/v). The following Fmoc amino acids were used: Fmoc-Ile-OH, Fmoc-Thr-OH (used without side chain protection), and Fmoc-Trp(Boc)-OH. After coupling of Fmoc-Trp(Boc)-OH esterification of the Thr side chain was achieved by treating the resin-bound peptide with Alloc-Gly-OH (1.20 g, 7.5 mmol, 15 eq.), DIC (1.2 mL, 7.5 mmol, 15 eq.) and DMAP (30 mg, 0.25 mmol, 0.5 eq.) in 16 mL DCM:DMF (3:1, v/v) for 18 h under nitrogen. The resin was treated with Pd(PPh₃)₄ (150 mg, 0.15 mmol, 0.3 eq.) and PhSiH₃ (1.5 mL, 15 mmol, 30 eq.) in DCM (ca. 30 mL) under nitrogen for 1 hour. The resin was subsequently washed with DCM (5×20 mL), followed by a solution of diethyldithiocarbamic acid trihydrate sodium salt (5 mg mL⁻¹ in DMF, 5×20 mL), and DMF (5×20 mL). The peptide was then cyclized using BOP (442 mg, 1.0 mmol, 4 eq.) and DiPEA (0.35 mL, 2.0 mmol, 8 eq.) for 2 h in 10 mL DMF under nitrogen flow. The remaining linear N-terminal section of the peptide was then synthesized using the standard SPPS protocol mentioned above. The following Fmoc amino acids were used: Fmoc-D-Ser(tBu)-OH, Fmoc-D-Tyr(tBu)-OH, Fmoc-D-Trp(Boc)-OH, Fmoc-D-Orn(Boc)-OH, Fmoc-L-Orn(Boc)-OH, Fmoc-Gly-OH. Following the coupling of the last amino acid the two batches of resin were divided into batches of 0.1 mmol and treated according to the conditions described in the table below. Final deprotections were carried out by treating the resins with TFA:TIS:H₂O (95:2.5:2.5, 5 mL) for 90 min. The reaction mixtures were filtered through cotton, the filtrates precipitated in MTBE:petroleum ether (1:1) and the resulting precipitates washed once more with MTBE:petroleum ether (1:1). The crude cyclic peptides were lyophilized from tBuOH:H₂O (1:1) and purified with reverse phase HPLC (protocol below). Pure fractions were pooled and lyophilized to yield laterocidine analogues listed in the table below in >95% purity as a white powder. Peptides were purified using a BESTA-Technik system with a Dr. Maisch Reprosil Gold 120 C18 column (25×250 mm, 10 m) and equipped with a ECOM Flash UV detector monitoring at 214 nm and 254 nm. The following solvent system, at a flow rate of 12 mL/min, was used: solvent A, 0.1% TFA in water/acetonitrile 95/5; solvent B, 0.1% TFA in water/acetonitrile 5/95. Gradient elution was as follows: 100:0 (A/B) for 5 min, 100:0 to 30:70 (A/B) over 50 min, 30:70 to 0:100 (A/B) for 3 min, then reversion back to 100:0 (A/B) over 1 min, 100:0 (A/B) for 5 min.

| Lipid | | Yield | |
|---|---|---|---|
| length | Conditions | (%) | HRMS data |
| NH₂ | Fmoc deprotection | 2.3 | [M + 2H]²⁺ calculated for C₆₉H₉₇N₁₉O₁₇ |
| | | | 732.8728; found 732.8731 |
| C₂ | Fmoc deprotection; Acetic anhydride | 0.9 | [M + 2H]²⁺ calculated for C₇₁H₉₉N₁₉O₁₈ |
| | (47 μL, 0.5 mmol, 5 eq.), DiPEA (87 | | 753.8781; found 753.8783 |
| | μL, 0.5 mmol, 5 eq.) in DMF (5 mL), | | |
| | 2 h under nitrogen | | |
| C₄ | Fmoc deprotection; Lipid coupling | 2.9 | [M + 2H]²⁺ calculated for C₇₃H₁₀₃N₁₉O₁₈ |
| | (Butyric acid) | | 767.8937; found 767.8944 |
| C₆ | Fmoc deprotection; Lipid coupling | 1.7 | [M + 2H]²⁺ calculated for C₇₅H₁₀₇N₁₉O₁₈ |
| | (Hexanoic acid) | | 781.9094; found 781.9097 |
| C₈ | Fmoc deprotection; Lipid coupling | 1.9 | [M + 2H]²⁺ calculated for C₇₇H₁₁₁N₁₉O₁₈ |
| | (Octanoic acid) | | 795.9250; found 795.9254 |
| C₁₀ | Fmoc deprotection; Lipid coupling | 2.8 | [M + 2H]²⁺ calculated for C₇₉H₁₁₅N₁₉O₁₈ |
| | (Decanoic acid) | | 809.9407; found 809.9410 |
| C₁₂ | Fmoc deprotection; Lipid coupling | 0.4 | [M + 2H]²⁺ calculated for C₈₁H₁₁₉N₁₉O₁₈ |
| | (Lauric acid) | | 823.9563; found 823.9567 |
| C₁₄ | Fmoc deprotection; Lipid coupling | 1.0 | [M + 2H]²⁺ calculated for C₈₃H₁₂₃N₁₉O₁₈ |
| | (Myristic acid) | | 851.9876; found 851.9881 |
| C₁₆ | Fmoc deprotection; Lipid coupling | 1.1 | [M + 2H]²⁺ calculated for C₈₅H₁₂₇N₁₉O₁₈ |
| | (Palmitic acid) | | 837.9720; found 837.9722 |

### 2.1.4. Solid Phase Peptide Synthesis of Linear Laterocidine Analogues—Formula (II)

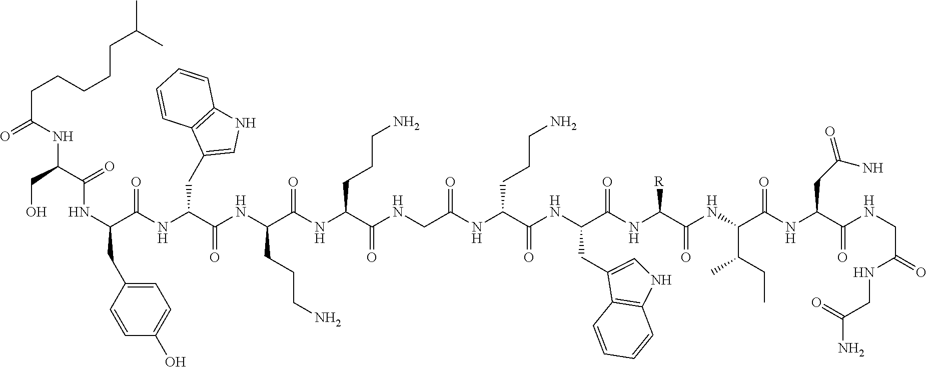

Linear laterocidine analogues were synthesised using automated synthesis on a CEM Liberty Blue Microwave Peptide synthesizer. Solid phase synthesis was carried out on a 0.25 mmol scale using Fmoc chemistry on Rink amide MBHA resin (0.67 mmol g⁻¹). Commercially available protected amino acids were used in 0.2 M solutions in DMF with couplings carried out using DIC as the activator, Oxyma as base and heated to 75° C. for 15 seconds and then to 90° C. for 110 seconds (Fmoc-AA:DIC:Oxyma, 1:5:5:5 molar eq.). For residue 9 (shown as including an R group in the structure above), protected forms of Thr, Dap(Alloc), and (2S,3R)-2-amino-3-azidobutanoic acid were used to prepare analogues. Fmoc residues were initially deprotected using a 20% solution of piperidine (75° C., 16 seconds), followed by a subsequent deprotection (90° C., 50 seconds). On completion, the resin was removed from the peptide synthesizer, washed with DCM (3×3 mL) before a cleavage cocktail of TFA, TIPS and H₂O (10 mL, 95:2.5:2.5) was added and agitated for 90 min. The reaction mixture was filtered through cotton, the filtrate was precipitated in MTBE:petroleum ether (1:1) and the resulting precipitate washed once more with MTBE:petroleum ether (1:1). The crude cyclic peptide was lyophilized from tBuOH:H₂O (1:1) and purified by RP-HPLC. The fractions containing the product were pooled, concentrated under vacuum, frozen and lyophilised. Yields ranged between 5% and 29%.

Further linear analogues of laterocidine were synthesised using the following automated solid-phase synthesis protocol: Peptides were made on Rink amide MBHA resin using a CEM Liberty Blue automated peptide synthesizer with microwave irradiation. Couplings were performed at 0.5 M concentration using 5 eq. of amino acid, 5 eq. of N,N′-diisopropylcarbodiimide (DIC) and 5 eq. of ethyl cyanohydroxyiminoacetate (Oxyma). Fmoc group removal was performed using piperidine:DMF (1:4, v/v). A detailed overview of the automated protocols can be found below. The resin was swollen in 10 ml of DMF for 300 s prior to the first coupling.

Normal Coupling protocol:

| Step | Function | Duration/Temperature |
|---|---|---|
| 1 | Deprotection N-terminus | 15 s at 75° C. then 50 s at 90° C. |
| 2 | Wash (DMF) | RT |
| 3 | Wash (DMF) | RT |
| 4 | Wash (DMF) | RT |
| 5 | Coupling amino acid | 15 s at 75° C. then 110 s at 90° C. |

Final deprotection N-terminus:

| Step | Function | Duration/Temperature |
|---|---|---|
| 1 | Deprotection N-terminus | 15 s at 75° C. then 50 s at 90° C. |
| 2 | Wash (DMF) | RT |
| 3 | Wash (DMF) | RT |
| 4 | Wash (DMF) | RT |

Following the final Fmoc removal step, the resin was removed from the CEM Liberty Blue and washed with DCM and DMF before isopelargonic acid (2 eq.) was coupled manually using benzotriazol−1-yloxytris(dimethylamino)phosphonium hexafluorophosphate (BOP) (2 eq.) and N,N-diisopropylethylamine (DiPEA) (4 eq.) in DMF overnight at RT, under nitrogen flow._Final sidechain deprotection and cleavage from the resin was carried out by treating the resin with TFA:TIPS:H₂O (95:2.5:2.5) for 90 min. The reaction mixture was filtered through cotton, the filtrate was precipitated in MTBE:petroleum ether (1:1) and centrifuged (4500 rpm, 5 min). The pellet was then resuspended in MTBE:petroleum ether (1:1) and centrifuged again (4500 rpm, 5 min). Finally the pellet containing the crude lipopeptide was dissolved in tBuOH:H₂O (1:1) and lyophilized overnight. Peptides were purified using a BESTA-Technik system with a Dr. Maisch Reprosil Gold 120 C18 column (25×250 mm, 10 μm) and equipped with a ECOM Flash UV detector monitoring at 214 nm and 254 nm. The following solvent system, at a flow rate of 12 mL/min, was used: solvent A, 0.1% TFA in water/acetonitrile 95/5; solvent B, 0.1% TFA in water/acetonitrile 5/95. Gradient elution was as follows: 100:0 (A/B) for 5 min, 100:0 to 50:50 (A/B) over 50 min, 50:50 to 0:100 (A/B) for 3 min, then reversion back to 100:0 (A/B) over 1 min, 100:0 (A/B) for 5 min.

### 2.1.5. Linear Laterocidine Alanine Scan

The alanine scan was performed using the automated solid-phase peptide synthesis protocol mentioned above by sequentially substituting every positon in linear laterocidine with either D or L-alanine. Lipidation, final deprotection, resin cleavage and HPLC purification were also performed as described in the general protocol. Linear laterocidine analogues listed in the table below were determined to be >95% purity by HPLC.

| | | Yield | |
|---|---|---|---|
| Compound | Peptide sequence | (%) | HRMS data |
| Ala₁ (D- | FA-aywoOGoWTINGG- | 34 | [M + 2H]²⁺ calculated for C₇₈H₁₁₆N₂₀O₁₇ |
| Ser→D- | CONH₂ (SEQ ID No: 1) | | 803.4487; found 803.4487 |
| Ala) | | | |
| Ala₂ (D- | FA-sawoOGoWTINGG- | 31 | [M + 2H]²⁺ calculated for C₇₂H₁₁₂N₂₀O₁₇ |
| Tyr→D- | CONH₂ (SEQ ID No: 2) | | 765.4330; found 765.4331 |
| Ala) | | | |
| Ala₃ (D- | FA-syaoOGoWTINGG- | 37 | [M + 2H]²⁺ calculated for C₇₀H₁₁₁N₁₉O₁₈ |
| Trp→D- | CONH₂ (SEQ ID No: 3) | | 753.9250; found 753.9254 |
| Ala) | | | |
| Ala₄ (D- | FA-sywaOGoWTINGG- | 27 | [M + 2H]²⁺ calculated for C₇₆H₁₁₁N₁₉O₁₈ |
| Orn→D- | CONH₂ (SEQ ID No: 4) | | 789.9250; found 789.9256 |
| Ala) | | | |
| Ala₅ | FA-sywoAGoWTINGG- | 30 | [M + 2H]²⁺ calculated for C₇₆H₁₁₁N₁₉O₁₈ |
| (Orn→Ala) | CONH₂ (SEQ ID No: 5) | | 789.9250; found 789.9255 |
| Ala₆ | FA-sywoOAoWTINGG- | 11 | [M + 2H]²⁺ calculated for C₇₉H₁₁₈N₂₀O₁₈ |
| (Gly→Ala) | CONH₂ (SEQ ID No: 6) | | 818.4539; found 818.4544 |
| Ala₇ (D- | FA-sywoOGaWTINGG- | 28 | [M + 2H]²⁺ calculated for C₇₆H₁₁₁N₁₉O₁₈ |
| Orn→D- | CONH₂ (SEQ ID No: 7) | | 789.9250; found 789.9256 |
| Ala) | | | |
| Ala₈ | FA-sywoOGoATINGG- | 31 | [M + 2H]²⁺ calculated for C₇₀H₁₁₁N₁₉O₁₈ |
| (Trp→Ala) | CONH₂ (SEQ ID No: 8) | | 753.9250; found 753.9254 |
| Ala₉ | FA-sywoOGoWAINGG- | 43 | [M + 2H]²⁺ calculated for C₇₇H₁₁₄N₂₀O₁₇ |
| (Thr→Ala) | CONH₂ (SEQ ID No: 9) | | 796.4408; found 796.4409 |
| Ala₁₀ | FA-sywoOGoWTANGG- | 34 | [M + 2H]²⁺ calculated for C₇₅H₁₁₀N₂₀O₁₈ |
| (Ile→Ala) | CONH₂ (SEQ ID No: 10) | | 790.4226; found 790.4229 |
| Alan₁₁ | FA-sywoOGoWTIAGG- | 6 | [M + 2H]²⁺ calculated for C₇₇H₁₁₅N₁₉O₁₇ |
| (Asn→Ala) | CONH₂ (SEQ ID No: 11) | | 789.9432; found 789.9429 |
| Ala₁₂ | FA-sywoOGoWTINAG- | 29 | [M + 2H]²⁺ calculated for C₇₉H₁₁₈N₂₀O₁₈ |
| (Gly→Ala) | CONH₂ (SEQ ID No: 12) | | 818.4539; found 818.4546 |
| Ala₁₃ | FA-sywoOGoWTINGA- | 32 | [M + 2H]²⁺ calculated for C₇₉H₁₁₈N₂₀O₁₈ |
| (Gly→Ala) | CONH₂ (SEQ ID No: 13) | | 818.4539; found 818.4541 |

FA = isopelargonic acid, alanines in bold, D-amino acids in lower case

### 2.1.6. Linear Laterocidine Position 9 Analogues

The position 9 screen was performed using the automated solid-phase peptide synthesis, lipidation, final deprotection, resin cleavage and HPLC purification protocol mentioned above. Linear laterocidine analogues listed in the table below were determined to be >95% purity by HPLC.

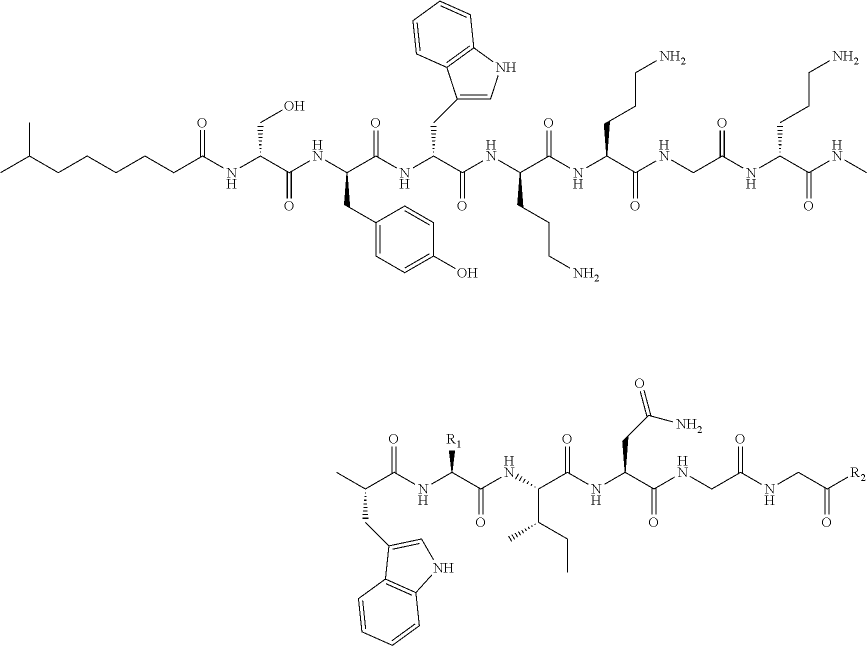

| | R₁ (full | | Yield | |
|---|---|---|---|---|
| Compound | amino acid) | R₂ | (%) | HRMS/LCMS data |
| Linear | Thr | OH | 19 | [M + 2H]²⁺ calculated for |
| Laterocidine | | | | C₇₈H₁₁₅N₁₉O₁₉ 811.9381; found |
| (Thr₉-COOH) | | | | 811.9386 (HRMS) |
| Thr₉-CONH₂ | Thr | NH₂ | 29 | [M + 2H]²⁺ calculated for |
| | | | | C₇₈H₁₁₆N₂₀O₁₈ 811.4461; |
| | | | | found 811.4464 (HRMS) |
| Dap(Alloc)₉- | Dap(Alloc) | OH | 19 | [M + 2H]²⁺ calculated for |
| COOH | | | | C₈₁H₁₁₈N₂₀O₂₀ 846.4489; found |
| | | | | 846.4489 (HRMS) |
| Dap(Alloc)₉- | Dap(Alloc) | NH₂ | 15 | [M + 2H]²⁺ calculated for |
| CONH₂ | | | | C₈₁H₁₁₉N₂₁O₁₉ 845.9569; found |
| | | | | 845.9574 (HRMS) |
| Dap(Alloc)₉- | Dap(Alloc) | OAllyl | 8.0 | [M + 2H]²⁺ calculated for |
| COOAll | | | | C₈₄H₁₂₂N₂₀O₂₀ 866.4645; found |
| | | | | 866.4648 (HRMS) |
| Ser₉-CONH₂ | Ser | NH₂ | 22 | [M + 2H]²⁺ calculated for |
| | | | | C₇₇H₁₁₄N₂₀O₁₈ 804.4383; found |
| | | | | 804.4386 (HRMS) |
| Dap₉-CONH₂ | Dap | NH₂ | 21 | [M + 2H]²⁺ calculated for |
| | | | | C₇₇H₁₁₅N₂₁O₁₇ 803.9463; found |
| | | | | 803.9465 (HRMS) |
| Leu₉-CONH₂ | Leu | NH₂ | 10 | [M + H]⁺ calculated |
| | | | | C₈₀H₁₂₀N₂₀O₁₇ 1633.92; found |
| | | | | 1633.75 (LCMS) |
| Phe₉-CONH₂ | Phe | NH₂ | 22 | [M + H]⁺ calculated for |
| | | | | C₈₃H₁₁₈N₂₀O₁₇ 1667.91; found |
| | | | | 1667.70 (LCMS) |
| Met₉-CONH₂ | Met | NH₂ | 17 | [M + H]⁺ calculated for |
| | | | | C₇₉H₁₁₈N₂₀O₁₇S 1651.88; found |
| | | | | 1651.70 (LCMS) |
| Trp₉-CONH₂ | Trp | NH₂ | 20 | [M + H]⁺ calculated for |
| | | | | C₈₅H₁₁₉N₂₁O₁₇ 1706.92; found |
| | | | | 1706.65 (LCMS) |
| Asn₉-CONH₂ | Asn | NH₂ | 20 | [M + H]⁺ calculated for |
| | | | | C₇₈H₁₁₅N₂₁O₁₈ 1634.88; found |
| | | | | 1634.55 (LCMS) |
| Gln₉-CONH₂ | Gln | NH₂ | 17 | [M + H]⁺ calculated for |
| | | | | C₇₉H₁₁₇N₂₁O₁₈ 1648.90; found |
| | | | | 1648.60 (LCMS) |
| Glu(OAll)₉- | Glu(OAllyl) | NH₂ | 14 | [M + H]⁺ calculated for |
| CONH₂ | | | | C₈₂H₁₂₀N₂₀O₁₉ 1689.91; found |
| | | | | 1689.85 (LCMS) |
| MeDap(N3)₉- CONH₂ | 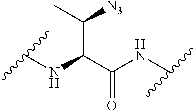 [SMILES: *NC(=O)[C@@H](N*)[C@@H](C)N=[N+]=[N-]] | NH₂ | 13 | [M + 2H]²⁺ calculated for C₇₈H₁₁₅N₂₃O₁₇ 823.9494; found 823.9495 (HRMS) |

### 2.1.7. Laterocidine N/C-Termini Analogues

The truncation study was performed using the automated solid-phase peptide synthesis, lipidation, final deprotection, resin cleavage and HPLC purification protocol mentioned above. Linear laterocidine analogues listed in the table below were determined to be >95% purity by HPLC.

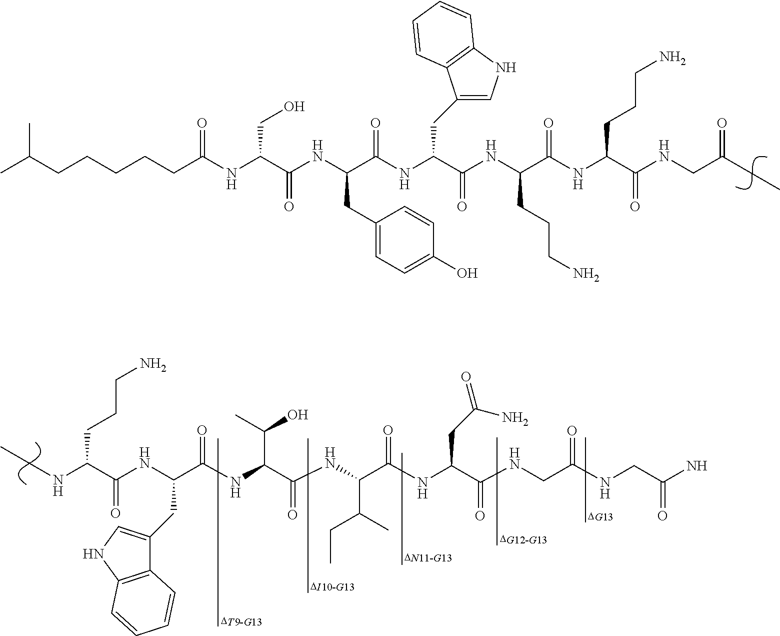

| | | Yield | |
|---|---|---|---|
| Compound | Peptide sequence | (%) | HRMS data |
| ΔG13 | FA-sywoOGoWTING- | 29 | [M + 2H]²⁺ calculated for C₇₆H₁₁₃N₁₉O₁₇ |
| | CONH₂ (SEQ ID No: 14) | | 782.9354; found 782.9358 |
| ΔG12-G13 | FA-sywoOGoWTIN- | 24 | [M + 2H]²⁺ calculated for C₇₄H₁₁₀N₁₈O₁₆ |
| | CONH₂ (SEQ ID No: 15) | | 754.4247; found 754.4248 |
| ΔN11- G13 | FA-sywoOGoWTI- | 20 | [M + 2H]²⁺ calculated for C₇₀H₁₀₄N₁₆O₁₄ |
| | CONH₂ (SEQ ID No: 16) | | 697.4032; found 697.4037 |
| ΔI10- G13 | FA-sywoOGoWT-CONH₂ | 29 | [M + 2H]²⁺ calculated for C₆₄H₉₃N₁₅O₁₃ |
| | (SEQ ID No: 17) | | 640.8612; found 640.8614 |
| ΔT9-G13 | FA-sywoOGoW-CONH₂ | 14 | [M + H]⁺ calculated for C₆₀H₈₆N₁₄O₁₁ |
| | (SEQ ID No: 18) | | 1179.6673; found 1179.6670 |

FA = isopelargonic acid, D-amino acids in lower case

### 2.1.8. Other Laterocidine Linear Analogues

The combinatorial study was performed using the automated solid-phase peptide synthesis, lipidation, final deprotection, resin cleavage and HPLC purification protocol mentioned above. Linear laterocidine analogues listed in the tables below were determined to be >95% purity by HPLC.

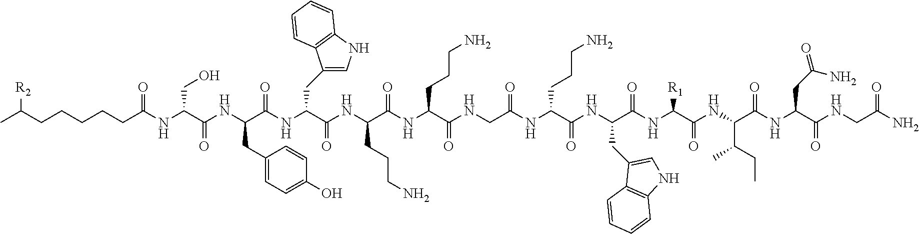

| | | | Yield | |
|---|---|---|---|---|
| Compound | R₁ | R₂ | (%) | LCMS data |
| ΔG13- | Dap(Alloc) | CH₃ | 10 | [M + H]⁺ calculated for C₇₉H₁₁₆N₂₀O₁₈ |
| Dap(Alloc)9- | | | | 1633.88; found 1633.60 |
| isoC9 | | | | |
| ΔG13- | Dap(Alloc) | H | 6 | [M + H]⁺ calculated for C₇₈H₁₁₄N₂₀O₁₈ |
| Dap(Alloc)9-C8 | | | | 1619.87; found 1620.45 |
| ΔG13-Ser9- | Ser | CH₃ | 18 | [M + H]⁺ calculated for C₇₅H₁₁₁N₁₉O₁₇ |
| isoC9 | | | | 1550.85; found 1550.50 |
| ΔG13-Ser9-C8 | Ser | H | 25 | [M + H]⁺ calculated for C₇₄H₁₀₉N₁₉O₁₇ |
| | | | | 1536.83; found 1536.40 |

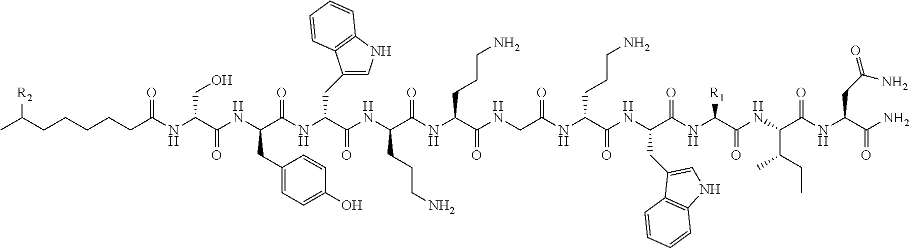

| | | | Yield | |
|---|---|---|---|---|
| Compound | R₁ | R₂ | (%) | LCMS data |
| ΔG12-G13- | Dap(Alloc) | CH₃ | 13 | [M + H]⁺ calculated for C₇₇H₁₁₃N₁₉O₁₇ |
| Dap(Alloc)9- | | | | 1576.86; found 1576.60 |
| isoC9 | | | | |
| ΔG12-G13- | Dap(Alloc) | H | 15 | [M + H]⁺ calculated for C₇₆H₁₁₁N₁₉O₁₇ |
| Dap(Alloc)9-C8 | | | | 1562.85; found 1562.60 |
| ΔG12-G13- | Ser | CH₃ | 28 | [M + H]⁺ calculated for C₇₃H₁₀₈N₁₈O₁₆ |
| Ser9-isoC9 | | | | 1493.83; found 1493.50 |
| ΔG12-G13- | Ser | H | 19 | [M + H]⁺ calculated for C₇₂H₁₀₆N₁₈O₁₆ |
| Ser9-C8 | | | | 1479.81; found 1480.45 |

### 2.1.9. Dap9-Relacidamide A

2-Chlorotrityl resin (2-CT) (5.0 g, 1.60 mmol g⁻¹) was loaded with Fmoc-Gly-OH. Resin loading was determined to be 0.67 mmol g⁻¹. The linear peptide was assembled manually on a 0.1 mmol scale under nitrogen flow via standard Fmoc solid-phase peptide synthesis (SPPS) (1 h couplings, resin:Fmoc-AA:BOP:DiPEA, 1:4:4:8 molar eq.). DMF (3 mL) was used as solvent and Fmoc deprotections (2 min then 10 min) were carried out with 3 mL piperidine:DMF (1:4, v/v). The following Fmoc amino acids were used: Fmoc-D-Ser(tBu)-OH, Fmoc-D-Tyr(tBu)-OH, Fmoc-D-Trp(Boc)-OH, Fmoc-L-Orn(Boc)-OH, Fmoc-L-Orn(Boc)-OH, Fmoc-Gly-OH, Fmoc-Trp(Boc)-OH, Fmoc-Dap(Alloc)-OH, Fmoc-Ile-OH and Fmoc-Ser(tBu)-OH. Following the final Fmoc removal step, 4-methylhexanoic acid (26 mg, 0.2 mmol, 2 eq.) was coupled using BOP (88 mg, 0.2 mmol, 2 eq.) and DiPEA (87 μL, 0.4 mmol, 4 eq.) in 3 mL of DMF overnight, under nitrogen flow. The resin was then treated three times with Pd(PPh₃)₄ (30.mg, 0.03 mmol, 0.3 eq.) and PhSiH₃ (0.30 mL, 3 mmol, 30 eq.) in DCM (ca. 7 mL) under nitrogen for 2 hours with washing in between with DCM (5×10 mL), followed by a solution of diethyldithiocarbamic acid trihydrate sodium salt (5 mg mL⁻¹ in DMF, 5×10 mL), and DMF (5×10 mL). The peptide was cleaved off the resin by treating it with HFIP:DCM (1:4, v/v, 10 mL) for 1 hour and rinsed with additional HFIP:DCM and DCM. The combined washings were then evaporated to yield the linear protected peptide with a free C-terminus and Dap amino sidechain. The partially protected peptide was dissolved in DCM (100 mL), treated with BOP (88 mg, 0.2 mmol, 2 eq.) and DiPEA (87 μL, 0.4 mmol, 4 eq.) and the solution was stirred overnight under nitrogen atmosphere. The reaction mixture was concentrated and directly treated with TFA:TIS:H₂O (95:2.5:2.5, 5 mL) for 90 min. The reaction mixture was subsequently filtered through cotton, the filtrate was precipitated in MTBE:petroleum ether (1:1) and the resulting precipitate washed once more with MTBE:petroleum ether (1:1). The crude cyclic peptide was lyophilized from tBuOH:H₂O (1:1) and purified using reverse phase HPLC (protocol below). Pure fractions were pooled and lyophilized to yield Dap9-laterocidamide in >95% purity as a white powder. Yield 19 mg, 12% yield over 30 steps. [M+H]⁺ calculated for C₇₄H₁₀₇N₁₉O₁₇ 1534.82 found (LCMS) 1534.55. Peptides were purified using a BESTA-Technik system with a Dr. Maisch Reprosil Gold 120 C18 column (25×250 mm, 10 μm) and equipped with a ECOM Flash UV detector monitoring at 214 nm and 254 nm. The following solvent system, at a flow rate of 12 mL/min, was used: solvent A, 0.1% TFA in water/acetonitrile 95/5; solvent B, 0.1% TFA in water/acetonitrile 5/95. Gradient elution was as follows: 100:0 (A/B) for 5 min, 100:0 to 50:50 (A/B) over 50 min, 50:50 to 0:100 (A/B) for 3 min, then reversion back to 100:0 (A/B) over 1 min, 100:0 (A/B) for 5 min.

2.1.10. Relacidine Analogues

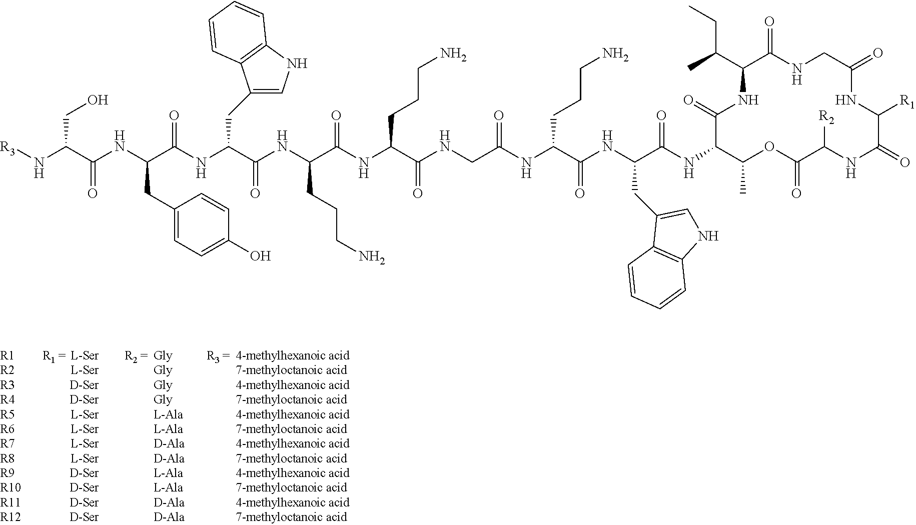

2-Chlorotrityl chloride resin (2-CT) (5 g, 1.55 mmol g⁻¹) was loaded by overnight coupling via the free sidechain hydroxyl of Fmoc-L-Ser-OAll (2.84 g, 7.75 mmol, 1 eq.) or Fmoc-D-Ser-OAll (1.5 g, 7.75 mmol, 1 eq.) with DiPEA (1.4 mL, 7.75 mmol, 1 eq.) in 23 mL of DCM. The suspension was stirred under argon at 45° C. for 5 min. An additional volume of DiPEA (2.1 mL, 11.1 mmol, 1.5 eq.) was added, the suspension was stirred under argon at 45° C., overnight. After capping with MeOH (0.92 mL, 22.7 mmol, 3 eq.) and DiPEA (0.67 mL, 3.7 mmol, 0.5 eq.) for 15 min the resin loading was determined to be 0.37 mmol·g⁻¹ and 0.33 mmol·g⁻¹ for CTC-Fmoc-L-Ser-OAll and CTC-Fmoc-D-Ser-OAll respectively. Resin loaded with Fmoc-L-Ser-OAll (0.68 g, 0.25 mmol) or Fmoc-D-Ser-OAll (0.75 g, 0.25 mmol) were added to a manual SPPS cartridge and bubbled with nitrogen in DMF (5 mL, 30 min) to swell. Fmoc deprotections (1 min then 10 min) were carried out with 5 mL a solution of dry piperidine in DMF (1:5, v/v). The next 4 amino acids (Gly 11, Ile10, Thr9, Trp8) were coupled manually (1 h) under nitrogen flow via standard Fmoc solid-phase peptide synthesis (SPPS) (resin:Fmoc-AA:BOP:DiPEA, 1:4:4:8 molar eq.). The following Fmoc amino acids were used: Fmoc-Gly-OH, Fmoc-Ile-OH, Fmoc-Thr-OH (used without sidechain protection) and Fmoc-Trp(Boc)-OH. After coupling of Fmoc-Trp(Boc)-OH, esterification of the Thr side chain was achieved by treating the resin-bound peptide with Alloc-Gly-OH (0.60 g, 3.75 mmol, 15 eq.), DIC (0.59 mL, 3.75 mmol, 15 eq.), and DMAP (15 mg, 0.13 mmol, 0.5 eq.) in 8 mL DCM:DMF (3:1, v/v) for 18 h under argon, for relacidine A; or Alloc-L-Ala-OH or Alloc-D-Ala-OH (0.65 g, 3.75 mmol, 15 eq.), DIC (0.59 mL, 3.75 mmol, 15 eq.), and DMAP (15 mg, 0.13 mmol, 0.5 eq.) in 8 mL DCM:DMF (3:1, v/v) for 18 h under argon, for relacidine B. The resin was treated with Pd(PPh₃)₄ (75 mg, 0.075 mmol, 0.3 eq.), and PhSiH₃ (0.75 mL, 7.5 mmol, 30 eq.) in DCM (16.5 mL) under argon for 2 h. The resin was subsequently washed with dry DCM (5×5 mL×3 min), diethyldithiocarbamic acid trihydrate sodium salt in dry DMF (5 mg mL⁻¹, 5×5 mL×3 min), and dry DMF (5×5 mL×3 min). Subsequently, BOP (442 mg, 1 mmol, 4 eq.) and dry DiPEA (348 μL, 2 mmol, 8 eq.) were added to cyclize the peptide, the solution was bubbled with nitrogen for 1 h. The remaining N-terminal section of the peptide was then synthesized using the standard SPPS protocol mentioned above. The following Fmoc amino acids were used: Fmoc-D-Orn(Boc)-OH, Fmoc-Gly-OH, Fmoc-L-Orn(Boc)-OH, Fmoc-D-Trp(Boc)-OH, Fmoc-D-Tyr(tBu)-OH, and Fmoc-D-Ser(tBu)-OH. Following the coupling of the last amino acid, the resin was split into two batches of 0.125 mmol. The two batches were reacted in parallel with 4-methylhexanoic acid (34.mg, 0.25 mmol, 2 eq.) or 7-methyloctanoic acid (40 mg, 0.25 mmol, 2 eq.), BOP (221 mg, 0.5 mmol, 4 eq.), and DiPEA (174 μL, 1 mmol, 8 eq.) in dry DMF (3 mL), under nitrogen flow for 2 h. Final deprotections were carried out by treating the resins with TFA:H₂O:TIPS (95:2.5:2.5, 5 mL) for 90 min while shaking. The solution was then filtered through cotton. The filtrates were precipitated in MTBE:PE (1:1), the pellet was washed once more with MTBE:PE (1:1). The crude cyclic peptides were lyophilized from tBuOH:H₂O (1:1), and purified with reverse phase HPLC (protocol below). Pure fractions were pooled and lyophilized to yield all compounds in >95% purity. Peptides were purified on a BESTA-Technik system with a Dr. Maisch Reprosil Gold 120 C18 column (25×250 mm, 10 μm) and equipped with a ECOM Flash UV detector monitoring at 214 nm and 254 nm. The following solvent system, at a flow rate of 12 mL/min, was used: solvent A, 0.1% TFA in water/acetonitrile 95/5; solvent B, 0.1% TFA in water/acetonitrile 5/95. Gradient elution was as follows: 100:0 (A/B) for 5 min, 100:0 to 40:60 (A/B) over 50 min, 40:60 (A/B) to 0:100 (A/B) over 3 min, 0:100 (A/B) for 1 min, then reversion back to 100:0 (A/B) over 1 min.

| Compound | Yield (%) | LCMS data |
|---|---|---|
| R1 | 0.8 | [M + H]⁺ calculated for C₇₅H₁₀₈N₁₈O₁₈ 1549.82; found 1549.50 |
| R2 | 6.8 | [M + H]⁺ calculated for C₇₅H₁₀₈N₁₈O₁₈ 1549.82; found 1549.45 |
| R3 | 3.1 | [M + H]⁺ calculated for C₇₇H₁₁₂N₁₈O₁₈ 1576.85; found 1577.50 |
| R4 | 0.6 | [M + H]⁺ calculated for C₇₇H₁₁₂N₁₈O₁₈ 1576.85; found 1577.70 |
| R5 | 1.4 | [M + H]⁺ calculated for C₇₆H₁₁₀N₁₈O₁₈ 1563.83; found 1563.60 |
| R6 | 9.5 | [M + H]⁺ calculated for C₇₆H₁₁₀N₁₈O₁₈ 1563.83; found 1563.40 |
| R7 | 3.2 | [M + H]⁺ calculated for C₇₆H₁₁₀N₁₈O₁₈ 1563.83; found 1563.55 |
| R8 | 5.7 | [M + H]⁺ calculated for C₇₆H₁₁₀N₁₈O₁₈ 1563.83; found 1563.55 |
| R9 | 3.1 | [M + H]⁺ calculated for C₇₈H₁₁₄N₁₈O₁₈ 1591.86; found 1591.65 |
| R10 | 10.8 | [M + H]⁺ calculated for C₇₈H₁₁₄N₁₈O₁₈ 1591.86; found 1591.60 |
| R11 | 12.4 | [M + H]⁺ calculated for C₇₈H₁₁₄N₁₈O₁₈ 1591.86; found 1591.60 |
| R12 | 5.7 | [M + H]⁺ calculated for C₇₈H₁₁₄N₁₈O₁₈ 1591.86; found 1591.65 |

### 2.2. Brevicidine-Related Compounds

### 2.2.1. Brevicidine

To a flame dried 25 mL round bottom flask was added Fmoc-Ser-OAllyl (110 mg, 0.300 mmol) and dry dichloromethane (10.0 mL). 2-Chlorotrityl chloride resin (CTC) (1.00 g, 0.81 mmol g⁻¹) and DIPEA (210 μL, 1.20 mmol) were added. The suspension was stirred under argon for 48 hours, after which the resin was filtered through a manual SPPS vessel and washed with DCM (4×5 mL). The resin was then capped, and a small portion used to ascertain the loading. Standard Fmoc SPPS protocol was used to extend the peptide to the linear Fmoc-Thr-Ile-Gly-Ser peptide. A portion of this on-resin allyl protected tetrapeptide (78.0 mg. 0.01 mmol) was added to a manual SPPS vessel and bubbled in DCM (3 mL) with argon for 15 minutes. The solvent was discharged and an allyl deprotection solution of tetrakis (triphenylphosphine) palladium (23.0 mg, 20.0 μmol) and phenylsilane (12.0 μL, 0.100 mmol) in DCM and DMF (1:1, 2 mL) was added. The solution was bubbled with argon for 2 hours in darkness, after which the deprotection solution was discharged and the resin was washed with DMF (3×3 mL), 0.5% sodium diethyldithiocarbamate solution in DMF (4×5 m L), DMF (4×5 mL) and DCM (4×5 mL). The resin was dried under argon, then added to a 5 mL flame dried round bottom flask under argon. Dry dichloromethane (3 mL) was added and the suspension stirred for 15 minutes. An aliquot of benzoyl chloride stock (10.0 μL, 11.0 μmol), triethylamine (3.00 μL, 22.0 μmol) and DMAP (1 crystal) were added and the reaction mixture was stirred overnight at 60° C. The resin was then filtered through a manual SPPS vessel and washed with DMF (3×5 mL) and DCM (3×5 mL) before being dried under argon. A small sample was cleaved using a 2% TFA solution in DCM (1 mL). The cleavage cocktail was gently agitated for 1 hour and filtered through a glass wool plug. The filtrate was concentrated with a gentle stream of argon before being analysed by LC-MS ([M+H]⁺ calculated for C₃₀H₃₆N₄O₈ 581.2, found (LC-MS) 581.5). Following this modified Yamaguchi esterification, the brevicidine synthesis was completed by Fmoc SPPS. The crude mixture was and purified by HPLC method A and product-containing fractions were pooled and lyophilized to yield brevicidine as a white fluffy powder. HPLC retention time 23.3 min; [M+3H]³⁺ calculated for C₇₄H₁₀₆N₁₈O₁₇ 507.3, found (LC-MS) 507.8. ¹H NMR (600 MHz, d⁶-DMSO) also confirmed formation of brevicidine. Comparison of analysis of the synthesised compound with brevicidine isolated from natural sources provided an exact match.

### 2.2.2. Synthesis of Ent-Brevicidine (Ent-1)

To a flame dried 25 mL round bottom flask was added Fmoc-D-Ser-OAll (112 mg, 0.305 mmol) and dry dichloromethane (10.0 mL). 2-Chlorotrityl chloride resin (CT) (1.00 g, 0.81 mmol g⁻¹) and DIPEA (210 μL, 1.20 mmol) were added. The suspension was stirred under argon for 48 h at 45° C., after which the resin was filtered through a manual SPPS vessel and washed with DCM (4×5 mL). The resin was then capped and loading level of the first residue onto resin (0.25 mmol g⁻¹) was calculated as per the synthesis of brevicidine.

Standard Fmoc SPPS protocol was used to extend the peptide to the linear Fmoc-D-Thr-D-Ile-Gly-D-Ser peptide on a 0.1 mmol scale (400 mg) similar to the synthesis of brevicidine. Following SPPS of the tetrapeptide, an allyl deprotection solution of tetrakis (triphenylphosphine) palladium (231 mg, 0.200 mmol) and phenylsilane (123 μL, 0.998 mmol) in DCM and DMF (1:1, 2 mL) was added. The solution was bubbled with argon for 2 h in darkness, after which the deprotection solution was discharged and the resin was washed with DMF (3×3 mL), 0.5% sodium diethyldithiocarbamate solution in DMF (4×5 mL), DMF (4×5 mL) and DCM (4×5 mL). The resin was dried under argon, then added to a 5 mL flame dried round bottom flask under argon. Dry dichloromethane (3 mL) was added and the suspension stirred for 15 min. Benzoyl chloride (13.0 μL, 0.112 mmol), triethylamine (3.00 μL, 22.0 μmol) and catalytic DMAP (1 crystal) were added and the reaction mixture was stirred overnight at 60° C. The resin was then filtered through a manual SPPS vessel and washed with DMF (3×5 mL) and DCM (3×5 mL) before being dried under argon. A small sample was cleaved using a 2% TFA solution in DCM (1 mL). The cleavage cocktail was gently agitated for 1 h and filtered through a glass wool plug. The filtrate was concentrated with a gentle stream of argon before being analysed by LC-MS ([M+Na]⁺ calculated for C₃₀H₃₆N₄O₈ 603.2, found (LC-MS) 603.5). Following this modified Yamaguchi esterification, the synthesis of ent-brevicidine was completed using standard Fmoc SPPS protocols as described above after which 4-methylhexanoic acid was coupled to the N-terminus. The peptide was cleaved from resin, precipitated and purified by RP-HPLC (See HPLC purification of synthetic peptides) following the procedure for synthesising brevicidine. Yield: 3 mg, 2% over 28 steps. HPLC retention time 22.9 min (Method A); [M−H]⁻ calculated for C₇₄H₁₀₆N₁₈O₁₇ 1517.7910, found (HR-MS) 1517.7943.

2.2.3. Brevicidamide (Dap9-Brevicidine. S, Solution Phase Cyclization Route)—Formula (III)

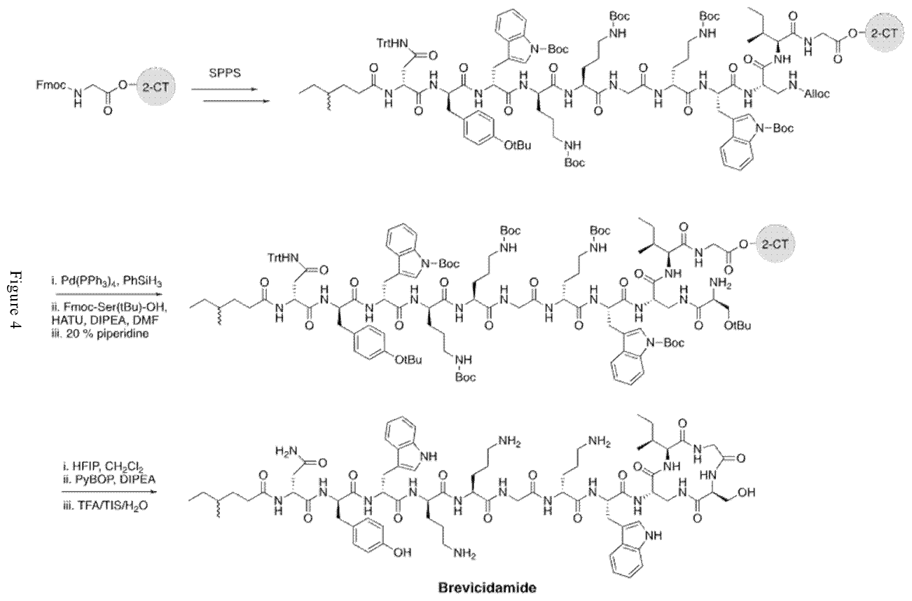

FIG. 4 depicts the solution phase cyclisation synthesis of brevicidamide. Brevicidamide was prepared on a 0.1 mmol scale from 2-CTC resin loaded with Fmoc-Gly. Fmoc SPPS was used to synthesise the linear peptide. The Alloc group was then removed by adding a solution of Pd(PPh₃)₄ (12 mg, 0.01 mmol) and phenyl silane (308 μL, 2.5 mmol) in DCM (6 mL) to the manual SPPS vessel and bubbling for 30 minutes with argon. The vessel was then flushed and the resin washed with DCM (3×5 mL), DMF (3×5 mL), a 0.5% solution of diethyldithiocarbamate in DMF (4×5 mL), DMF (3×5 mL) and DCM (3×5 mL). This was repeated. A coupling solution of Fmoc-Ser(tBu)-OH (230 mg, 0.6 mmol), HATU (230 mg, 0.6 mmol) and DIPEA (220 μL, 1.2 mmol) in DMF (6 mL) was added. The solution was bubbled for 1 hour with argon, before the resin was washed with DMF (3×5 mL) and the Fmoc group removed as per standard Fmoc SPPS protocol. The resin was dried under argon (230 mg) and then cleaved using 20% HFIP in DCM (10 mL) for 1 hour at room temperature. The solution was then filtered through a glass wool plug into a round bottom flask, and the filtrate concentrated under vacuum to yield a white solid (102 mg). A 1:1 solution of DMF and DCM (8.4 mL, peptide concentration: 5 mM) was added and the solution stirred until all solid had dissolved. PyBOP (44 mg, 84 μmol) and DIPEA (29 μL, 168 μmol) were added and the reaction stirred overnight while being monitored by TLC (5% MeOH/DCM with 1% acetic acid). After 24 hours the reaction mixture was concentrated under vacuum. A cleavage cocktail of TFA, TIPS and water (95:2.5:2.5, 10 mL) was added and the clear solution was stirred for 1.5 hours at 38° C. The clear, yellow solution was concentrated under vacuum. Diethyl ether was used to precipitate out the crude peptide which was then centrifuged and washed with more diethyl ether. The suspension was centrifuged and the crude pellet dissolved in a minimal amount of 1:1 acetonitrile and water solution with 0.1% TFA. The crude mixture was and purified by RP-HPLC and the fractions containing the product were pooled, concentrated under vacuum, frozen and lyophilised. Product obtained as a cream solid (19 mg, 30%); C₁₈ RP-HPLC retention time: 16.34 min; (M-2H)²⁻ calculated for C₇₃H₁₀₅N₁₉O₁₆ 750.8921, found (QTOF-ES-MS) 750.9122.

### 2.2.4. Brevicidamide (On-Resin Cyclization Route)—Formula (III)

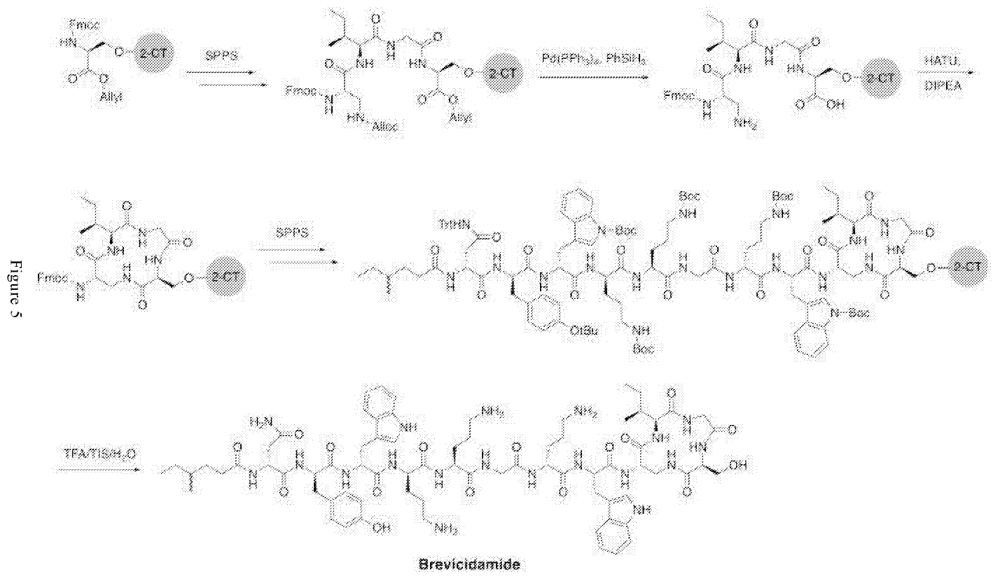

FIG. 5 depicts the on-resin cyclisation synthesis of brevicidamide.

To a flame dried 25 mL round bottom flask was added Fmoc-Ser-OAllyl (110 mg, 0.300 mmol) and dry dichloromethane (20.0 mL). 2-Chlorotrityl chloride resin (CTC) (1.00 g, 0.81 mmol g⁻¹) and DIPEA (210 μL, 1.20 mmol) were added. The suspension was stirred under argon at 45° C. for 24 hours, after which the resin was filtered through a manual SPPS vessel and washed with DCM (3×20 mL). The resin was then capped by adding a solution of methanol, DIPEA and DCM (20 mL, 10:5:85) and bubbled with argon for 1 hour. The solution was discharged and the resin was washed with DCM (3×5 mL) before being dried under a stream of argon. The resin loading was determined to be 0.2 mmol g⁻¹. Standard Fmoc SPPS protocol was used to extend the peptide to the linear Fmoc-Dap(Alloc)-Ile-Gly-Ser peptide. Specifically, resin (0.1 mmol) was added to a manual SPPS vessel and bubbled in DMF (3 mL) to swell. The solvent was discharged and the resin was bubbled in an Fmoc deprotection solution of 20% piperidine in DMF (3×3 mL, 2×1 min then 1×5 min) with argon. The resin was washed with DMF (3×3 mL) and a coupling solution of amino acid (6 equiv), HATU (6 equiv) and DIPEA (12 equiv) in DMF (3 mL) was added. The solution was then bubbled with argon for 1 hour, before the solution was discharged and the resin washed with DMF (3×3 mL). This process was repeated to obtain on-resin linear Fmoc-Dap(Alloc)-Ile-Gly-Ser. The dried resin was swollen by bubbling in DCM (5 mL) for 15 minutes. The solvent was discharged and an allyl deprotection solution of tetrakis (triphenylphosphine) palladium (426 mg, 0.400 mmol) and phenylsilane (246 μL, 2.00 mmol) in DCM and DMF (1:1, 4 mL) was added. The solution was bubbled with argon for 2 hours in darkness, after which the deprotection solution was discharged and the resin was washed with DMF (3×3 mL), 0.5% sodium diethyldithiocarbamate solution in DMF (4×5 mL), DMF (4×5 mL) and DCM (4×5 mL). The resin was swollen in DMF (5 mL) and a coupling solution of HATU (76.0 mg, 0.200 mmol) and DIPEA (70.0 μL, 4.02 mmol). The solution was bubbled with argon for 24 hours after which the solution was discharged and the resin washed with DMF (3×5 mL) then DCM (3×5 mL). Fmoc SPPS was used to extend the peptide fully before a global cleavage was carried out by adding the dried resin to a cleavage cocktail of TFA, TIPS and H₂O (10 mL, 95:2.5:2.5) and heated to 37° C. for 1 hour. The suspension was filtered through a glass wool plug and the filtrate concentrated under vacuum. Diethyl ether was used to precipitate out the crude peptide which was then centrifuged and washed with more diethyl ether. The suspension was centrifuged and the crude pellet dissolved in a minimal amount of 1:1 acetonitrile and water solution with 0.1% TFA. The crude mixture was and purified by RP-HPLC and the fractions containing the product were pooled, concentrated under vacuum, frozen and lyophilised to yield a fluffy, white powder (5 mg, 3% over 26 steps). [M+3H]³⁺ calculated for C₇₃H₁₀₅N₁₉O₁₆ 502.3, found (LC-MS) 502.7.

### 2.2.5. Synthesis of Ser9-Brevicidine (3)

The desired Fmoc-tetrapeptide was synthesized from Fmoc-Ser-OAll as described above, with Fmoc-Ser used in place of Fmoc-Thr. This resin-bound tetrapeptide (0.065 mmol, 0.14 mmol/g) was added to a manual SPPS vessel and bubbled with DMF (5 mL) for 15 min then the solvent was discharged. An allyl deprotection solution of tetrakis (triphenylphosphine) palladium (150 mg, 0.130 mmol) and phenylsilane (80.0 μL, 0.649 mmol) in DCM and DMF (1:1, 2 mL) was added. The solution was bubbled with argon for 2 h in darkness, after which the deprotection solution was discharged and the resin was washed with DMF (3×3 mL), 0.5% sodium diethyldithiocarbamate solution in DMF (4×5 mL), DMF (4×5 mL) and DCM (4×5 mL). The resin was dried under argon, then added to a 5 mL flame dried round bottom flask under argon. Dry dichloromethane (3 mL) was added and the suspension stirred for 15 min. Benzoyl chloride (8.00 μL, 68.9 μmol), triethylamine (20.0 μL, 0.143 mmol) and catalytic DMAP (1 crystal) were added and the reaction mixture was stirred overnight at 60° C. The resin was then filtered through a manual SPPS vessel and washed with DMF (3×5 mL) and DCM (3×5 mL) before being dried under argon. A small sample was cleaved using a 2% TFA solution in DCM (1 mL). The cleavage cocktail was gently agitated for 1 h and filtered through a glass wool plug. The filtrate was concentrated with a gentle stream of argon before being analysed by LC-MS ([M+H]⁺ calculated for C₃₀H₃₆N₄O₈ 567.2, found (LC-MS) 567.5). Following this modified Yamaguchi esterification, the synthesis of Ser9-brevicidine was completed using standard Fmoc SPPS protocols as described above. The peptide was cleaved from resin, precipitated and purified by RP-HPLC (See HPLC purification of synthetic peptides) following the procedure for synthesising brevicidine. Yield: 13 mg, 13% over 28 steps. HPLC retention time 22.1 min (Method A); [M+2H]²⁺ calculated for C₇₃H₁₀₄N₁₈O₇ 753.3986, found (HR-MS) 753.3980.

### 2.2.6. Synthesis of MeDap9-Brevicidine (7)

The desired Fmoc-tetrapeptide was synthesized from Fmoc-Ser-OAll as described above, with Fmoc-MeDap(Alloc)-OH (IUPAC name: (2S,3R)-2-[[(9H-fluoren-9-ylmethoxy)carbonyl]amino]-3-[[(2-propen-1-yloxy)carbonyl]amino]-butanoic acid) used in place of Fmoc-Thr. The synthesis of Fmoc-MeDap(Alloc)-OH was adapted from a previously reported literature precedent (R. Moran Ramallal, R. Liz & V. Gotor, J. Org. Chem., 2010, 75, 19, 6614-6624)—To a flame dried 25 mL round bottom flask was added Fmoc-L-Abu(3R—N₃)—OH (146 mg, 0.398 mmol) and 10% Pd/C (63 mg, 59.2 μmol). The flask was evacuated and a balloon of hydrogen was attached. Methanol (10 mL) was added and the suspension was stirred for 30 min before being filtered through a celite plug. The filtrate was concentrated under vacuum and re-dissolved in dichloromethane (10 mL). The solution was stirred at 0° C. then allyl chloroformate (51 μL, 0.478 mmol) and DIPEA (83 μL, 0.478 mmol) were added. The reaction was stirred at 0° C. for 3 h and concentrated under vacuum. The crude solid was subsequently purified by flash chromatography (1% MeOH in DCM with 1% acetic acid). The fractions containing product were pooled, concentrated and co-evaporated with toluene (3×10 mL) then chloroform (3×10 mL) to yield a white solid (70 mg, 41%); TLC:R_{f} 0.44 (10% MeOH in DCM); [α]_{D}²⁰: +31.9 (0.6, CHCl₃); ¹H NMR (400 MHz, CDCl₃): δ 7.74 (2H, d, J=7.5 Hz, Fmoc), 7.58 (2H, t, J=6.7 Hz, Fmoc), 7.38 (2H, t, J=7.3 Hz, Fmoc), 7.28 (2H, t, J=7.3 Hz, Fmoc), 6.09 (1H, d, J=7.7 Hz, —NH), 5.84 (1H, br, —OCH₂CH═CH₂), 5.32 (1H, d, J=6.7 Hz, —CHCH(CH₃)NH—) 5.23 (1H, d, J=17.1 Hz, —OCH₂CH═CH₂), 5.15 (1H, d, J=9.9 Hz, —OCH₂CH═CH₂), 4.52-4.35 (6H, m, —CHCH(CH₃)NH—, —CHCH(CH₃)NH—, Fmoc-CHCH), 4.19 (1H, t, J=6.9 Hz, Fmoc-CHCH₂), 1.23 (3H, br, —CHCH(CH₃)NH—); [M+H]⁺ calculated for, C₂₃H₂₄N₂O₆ 425.1707, found (LC-MS) 425.4.

This resin-bound tetrapeptide (0.05 mmol, 0.14 mmol/g) was added to a manual SPPS vessel and bubbled with DMF (5 mL) for 15 min then the solvent was discharged. An allyl deprotection solution of tetrakis (triphenylphosphine) palladium (231 mg, 0.200 mmol) and phenylsilane (123 μL, 0.998 mmol) in DCM and DMF (1:1, 6 mL) was added. The solution was bubbled with argon for 2 h in darkness, after which the deprotection solution was discharged and the resin was washed with DMF (3×3 mL), 0.5% sodium diethyldithiocarbamate solution in DMF (6×10 mL), DMF (4×5 mL) and DCM (4×5 mL). A solution of HATU (95.0 mg, 0.250 mmol) and DIPEA (87.0 μL, 0.499 mmol) in DMF (3 mL) and bubbled with argon overnight at 50° C. The coupling solution was discharged, and the resin was washed with DMF (3×5 mL) then DCM (3×5 mL) and dried under a stream of argon. A small sample was cleaved using a 2% TFA solution in DCM (1 mL). The cleavage cocktail was gently agitated for 1 h and filtered through a glass wool plug. The filtrate was concentrated with a gentle stream of argon before being analysed by LC-MS ([M+H]⁺ calculated for C₃₀H₃₆N₄O₈ 580.3, found (LC-MS) 580.6). The synthesis of MeDap9-brevicidine was completed using standard Fmoc SPPS protocols as described above. The peptide was cleaved from resin, precipitated and purified by RP-HPLC (See HPLC purification of synthetic peptides) following the procedure for synthesising brevicidine. Yield: 3 mg, 2% over 28 steps. HPLC retention time 21.2 min (Method A); [M+H]⁺ calculated for C₇₄H₁₀₇N₂₉O₁₆ 1518.8216, found (HR-MS) 1518.4032.

### 2.2.7. Brevicidine Lipid Analogues

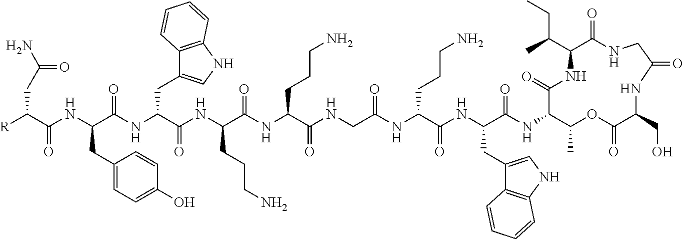

| Analogue | R |
|---|---|
| Brev-NH2 | NH₂ |
| Brev-C2 | C(O)CH₃ |
| Brev-C4 | C(O)(CH₂)₂CH₃ |
| Brev-C6 | C(O)(CH₂)₄CH₃ |
| Brev-C8 | C(O)(CH₂)₆CH₃ |
| Brev-C10 | C(O)(CH₂)₈CH₃ |
| Brev-C12 | C(O)(CH₂)₁₀CH₃ |
| Brev-C14 | C(O)(CH₂)₁₂CH₃ |
| Brev-C16 | C(O)(CH₂)₁₄CH₃ |

To a flame dried 25 mL round bottom flask was added Fmoc-Ser-OAllyl (110 mg, 0.300 mmol) and dry dichloromethane (10.0 mL). 2-Chlorotrityl chloride resin (CTC) (1.00 g, 0.81 mmol g⁻¹) and DIPEA (210 μL, 1.20 mmol) were added. The suspension was stirred under argon for 48 hours, after which the resin was filtered through a manual SPPS vessel and washed with DCM (4×5 mL). The resin was then capped, and a small portion used to ascertain the loading. Standard Fmoc SPPS protocol was used to extend the peptide to the linear Fmoc-Thr-Ile-Gly-Ser peptide. A portion of this on-resin allyl protected tetrapeptide (78.0 mg. 0.01 mmol) was added to a manual SPPS vessel and bubbled in DCM (3 mL) with argon for 15 minutes. The solvent was discharged and an allyl deprotection solution of tetrakis (triphenylphosphine) palladium (23.0 mg, 20.0 μmol) and phenylsilane (12.0 μL, 0.100 mmol) in DCM and DMF (1:1, 2 mL) was added. The solution was bubbled with argon for 2 hours in darkness, after which the deprotection solution was discharged and the resin was washed with DMF (3×3 mL), 0.5% sodium diethyldithiocarbamate solution in DMF (4×5 mL), DMF (4×5 mL) and DCM (4×5 mL). The resin was dried under argon, then added to a 5 mL flame dried round bottom flask under argon. Dry dichloromethane (3 mL) was added and the suspension stirred for 15 minutes. An aliquot of benzoyl chloride stock (10.0 μL, 11.0 μmol), triethylamine (3.00 μL, 22.0 μmol) and DMAP (1 crystal) were added and the reaction mixture was stirred overnight at 60° C. The resin was then filtered through a manual SPPS vessel and washed with DMF (3×5 mL) and DCM (3×5 mL) before being dried under argon. A small sample was cleaved using a 2% TFA solution in DCM (1 mL). The cleavage cocktail was gently agitated for 1 hour and filtered through a glass wool plug. The filtrate was concentrated with a gentle stream of argon before being analysed by LC-MS ([M+H]⁺ calculated for C₃₀H₃₆N₄O₈ 581.2, found (LC-MS) 581.5). Following this modified Yamaguchi esterification, the brevicidine synthesis was completed by Fmoc SPPS. The crude mixture was purified by HPLC.

### 2.2.8. Solid Phase Peptide Synthesis of Linear Brevicidine Analogues—Formula (V)

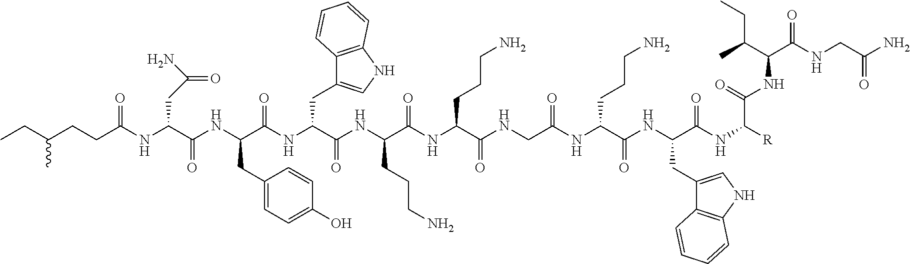

Linear brevicidine analogues were synthesised using automated synthesis on a CEM Liberty Blue Microwave Peptide synthesizer. Solid phase synthesis was carried out on a 0.025 mmol scale using Fmoc chemistry on Rink amide resin (0.45 mmol g⁻¹). Commercially available protected amino acids were used in 0.2 M solutions in DMF with couplings carried out using HATU as the activator and heated to 70° C. for 5 minutes. Fmoc residues were initially deprotected using a 20% solution of piperidine (70° C., 0.5 min), followed by a subsequent deprotection (70° C., 3 min). For residue 9 (shown as including an R group in the structure above), Fmoc-Dap(Alloc)-OH, Fmoc-Ser(tBu)-OH and Fmoc-Thr(tBu)-OH were used to prepare Ala, Dap(Alloc), Dap, Ser and Thr analogues. On completion, the resin was removed from the peptide synthesizer, washed with DCM (3×3 mL) and dried under vacuum for 15 min. A cleavage cocktail of TFA, TIPS and H₂O (10 mL, 95:2.5:2.5) was added and heated to 37° C. for 1 hour. The suspension was filtered through a glass wool plug and the filtrate concentrated under vacuum. Diethyl ether was used to precipitate out the crude peptide which was then centrifuged and washed with more diethyl ether. The suspension was centrifuged and the crude pellet dissolved in a minimal amount of 1:1 acetonitrile and water solution with 0.1% TFA. The crude mixture was and purified by RP-HPLC and the fractions containing the product were pooled, concentrated under vacuum, frozen and lyophilised. The analogues were isolated in 22-42% yield.

Corresponding C-terminal carboxylic acids were synthesised using substantially the same protocol but employing 2-chlorotrityl resin or Wang resin instead of Rink amide resin.

### 2.2.9. Linear Brevicidine Position 9 Analogues

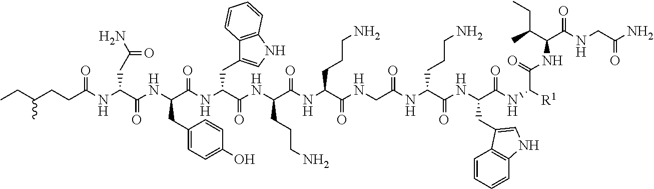

| Analogue | R¹ |
|---|---|
| Und(9-Dap)-NH₂ | CH₂NH₂ |
| Und(9-Ser)-NH₂ | CH₂OH |
| Und(9-Thr)-NH₂ | 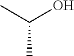 |
| Und(9-Leu)-NH₂ | 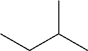 |
| Und(9-Phe)-NH₂ | Bn |
| Und(9-Trp)-NH₂ | 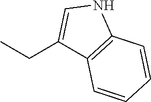 |
| Und(9-Asp)-NH₂ | CH₂COOH |
| Und(9-Gln)-NH₂ | (CH₂)₂CONH₂ |
| Und(9-AllGlu)-NH₂ | (CH₂)₂COO(allyl) |

Linear brevicidine analogues were synthesised using automated synthesis on a CEM Liberty Blue Microwave Peptide synthesizer. Solid phase synthesis was carried out on a 0.025 mmol scale using Fmoc chemistry on Rink amide resin (0.45 mmol g⁻¹). Commercially available protected amino acids were used in 0.2 M solutions in DMF with couplings carried out using HATU as the activator and heated to 70° C. for 5 minutes. Fmoc residues were initially deprotected using a 20% solution of piperidine (70° C., 0.5 min), followed by a subsequent deprotection (70° C., 3 min). On completion, the resin was removed from the peptide synthesizer, washed with DCM (3×3 mL) and dried under vacuum for 15 min. A cleavage cocktail of TFA, TIPS and H₂O (10 mL, 95:2.5:2.5) was added and heated to 37° C. for 1 hour. The suspension was filtered through a glass wool plug and the filtrate concentrated under vacuum. Diethyl ether was used to precipitate out the crude peptide which was then centrifuged and washed with more diethyl ether. The suspension was centrifuged and the crude pellet dissolved in a minimal amount of 1:1 acetonitrile and water solution with 0.1% TFA. The crude mixture was and purified by RP-HPLC and the fractions containing the product were pooled, concentrated under vacuum, frozen and lyophilised.

Corresponding C-terminal carboxylic acids were synthesised using substantially the same protocol but employing 2-chlorotrityl resin or Wang resin instead of Rink amide resin.

Similar methods were also used to synthesise the following compounds:

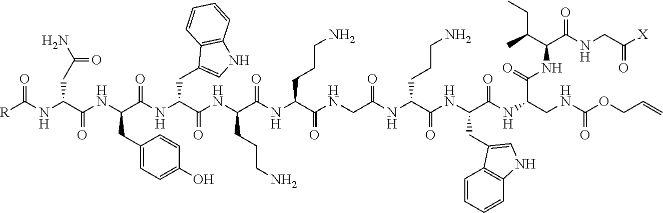

| Analogue | R | X |
|---|---|---|
| Undecacidin | 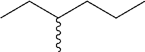 | OH |
| Oct-Undecacidin | 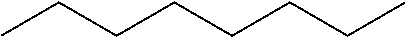 | OH |
| Undecacidamide | 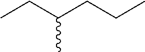 | NH₂ |
| Oct-Undecacidamide | 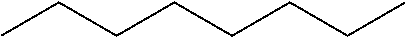 | NH₂ |

### 3. Biological Testing of Compounds

### 3.1. Antibacterial Activity

Minimum inhibitory concentration (MIC) was determined by broth microdilution according to CLSI guidelines. Blood agar plates were inoculated with glycerol stocks of Escherichia coli ATCC 25922, E. coli ATCC 25922 MCR-1, E. coli MCR-1 (clinical isolate from Utrecht Medical Center, NL), E. coli EQAS MCR-2 (clinical isolate from Wageningen University and Research, NL), Kiebsielia pneumoniae ATCC 11228, K. pneumoniae ATCC 13883, K. pneumonia 2048 (clinical isolate from Vrije Universiteit Amsterdam Medical Center, NL), K. pneumonia JS-123 (clinical isolate from from Utrecht Medical Center, NL), Acinetobacter baumannii ATCC 17961, A. baumannil ATCC 17978, A. baumannii 2018-006 (clinical isolate from Rijksinstituut voor Volksgezondheid en Milieu, NL), A. baumannii MDR (clinical isolate from Vrije Universiteit Amsterdam Medical Center, NL), Pseudomonas aeruginosa ATCC 27853, P. aeruginosa PAO1, P. aeruginosa NRZ-03961 (Reference strain from Das Nationale Referenzzentrum für gramnegative Krankenhauserreger, DE), P. aeruginosa M-120 (clinical isolate from Leiden University Medical Center, NL) and Staphylococcus aureus USA300 (clinical isolate from Texas Children's Hospitalm, USA). E. coli 25922 MCR1 was grown on LB agar supplemented with kanamycin. The inoculated agar plates were then incubated for 16 hours at 37° C. Individually grown colonies were subsequently used to inoculate 3 mL aliquots of TSB that were then incubated at 37° C. with shaking at 220 rpm. In parallel, the compounds to be assessed were serially diluted with Mueller-Hinton broth (MHB) in polypropylene 96-well plates (50 μL in each well). Once the OD₆₀₀ of the bacterial suspensions reached 0.5, the bacteria were diluted with MHB (final concentration 2×105 CFU mL⁻¹) and added to the microplates containing the test compounds (50 μL to each well, final volume: 100 μL). The well-plates were sealed with an adhesive membrane and after 16 hours of incubation at 37° C. with shaking at 220 rpm the wells were visually inspected for bacterial growth. MIC values were defined as the lowest concentration of the compound that prevented visible growth of bacteria.

### 3.1.1. In Vitro—Laterocidine, Laterocidamide, Brevicidine and Brevicidamide

The minimum inhibitory concentration (MIC) of a range of bacteria by the compounds laterocidine (natural product), laterocidamide (formula (I)), brevicidine (natural product), brevicidamide (formula (III)) and colistin (known antibiotic medication used as a last-resort treatment for multidrug-resistant Gram-negative infections) was assessed. The results are shown in the table below.

Minimum inhibitory concentration (MIC) of bacteria

| | Gram | MIC value μg/ml | | | | |
|---|---|---|---|---|---|---|
| Strain | Stain | Laterocidine | Laterocidamide | Brevicidine | Brevicidamide | Colistin |
| E. coli ATCC 25922 | − | 1 | 1 | 1-2 | 8-16 | 0.25-0.5 |
| E. coli ATCC 25922 MCR-1 | − | 2 | 1-2 | 2 | 16 | 2-4 |
| E. coli MCR-1 | − | 0.5 | 1 | 1-2 | 8-16 | 2-4 |
| E. coli EQAS MCR-2 | − | 1 | 1 | 2 | 8-16 | 2-4 |
| K. pneumoniae ATCC 11228 | − | 2 | 2 | 1-2 | 16-32 | ≤0.5 |
| K. pneumoniae ATCC 13883 | − | 1 | 2 | 1 | 32 | 1 |
| K. pneumoniae 2048 | − | 2 | 2 | 1-2 | 16-32 | 2 |
| K. pneumoniae JS-123 | − | 0.5 | ≤0.5 | 1 | 8 | ≤0.5 |
| A. baumannii ATCC 17961 | − | 0.5 | 4 | 2-4 | 4 | ≤0.5 |
| A. baumannii ATCC 17978 | − | 0.5 | 4 | 4 | 16 | ≤0.5 |
| A. baumannii 2018-006 | − | 2 | 4 | 4 | 8 | ≤0.5 |
| A. baumannii MDR | − | 1 | 4 | 4 | 8 | ≤0.5 |
| P. aeruginosa ATCC 27853 | − | ≤0.5 | 0.5-1 | ≤0.5 | 8 | 1 |
| P. aeruginosa PAO1 | − | 1 | 1-2 | 2 | 16-32 | ≤0.5 |
| P. aeruginosa NRZ-03961 | − | 1-2 | 2 | 2 | 16-32 | <0.5 |
| P. aeruginosa M-120 | − | 1 | 2-4 | 2-4 | 16 | 1 |
| S. aureus USA300 | + | 64 | >64 | 64 | >64 | >64 |

It can be seen that laterocidamide, a compound of formula (I) is effective against all Gram-negative bacteria tested. Whilst the activity of brevicidamide, a compound of formula (III), is lower than that of laterocidamide, this compound is still active against the Gram-negative bacteria. Brevicidamide is, therefore, still useful. Amide substitution of laterocidine was very well tolerated, providing a two-fold benefit of increased stability and comparable activity.

The compounds showed significantly lower activity against S. aureus, a Gram-positive bacteria. This shows that the compounds can selectively target Gram-negative bacteria.

Laterocidamide, of formula (I), was found to have at least comparable activity to that of the existing “last-resort” antibiotic colistin against most strains tested.

In the table above, “MCR” denotes that one of the MCR family of genes, which confers colistin resistance, is present in the bacterial strain. Thus, E. coli ATCC 25922 MCR-1, E. coli MCR-1 and E. coli EQAS MCR-2 are each colistin resistant. Laterocidamide, of formula (I), was found to have better activity than colistin against these strains. Therefore, laterocidamide may overcome some of the challenges faced due to antibiotic resistance.

It is proposed that the results for laterocidine would be similar to those for other similar peptides with 5-amino acid macrocycles, such as relacidine.

### 3.1.2. In Vitro—Ent-Laterocidine and Ent-Brevicidine

MIC values were determined for the enantiomers of laterocidine and brevicidine against a wide range of bacteria. The results are shown in the table below:

| | ent-Brevicidine | ent- Laterocidine |
|---|---|---|
| | ent-1 | ent-2 |
| E. coli ATCC 25922 | 16 | 8 |
| E. coli ATCC 25922 MCR-1 | 16 | 8-16 |
| E. coli MCR-1 | 16 | 8-16 |
| E. coli EQAS MCR-2 | 16-32 | 8-16 |
| K. pneumoniae ATCC 11228 | 32 | 16 |
| K. pneumoniae ATCC 13883 | 32 | 16 |
| K. pneumonia 2048 | 32 | 16 |
| K. pneumonia JS-123 | 8 | 4 |
| A. baumannii ATCC 17961 | 32 | 4 |
| A. baumannii ATCC 17978 | 16-32 | 4 |
| A. baumannii 2018-006 | 8-16 | 4 |
| A. baumannii MDR | 16 | 4-8 |
| P. aeruginosa ATCC 27853 | 16 | 8 |
| P. aeruginosa PAO1 | 32 | 16 |
| P. aeruginosa NRZ-03961 | 32 | 16 |
| P. aeruginosa M-120 | 16 | 16 |
| S. aureus USA300 | >64 | 64 |

Minimum inhibitory concentrations reported in μg/mL.

The results show that the enantiomers of brevicidine and laterocidine are active against a wide range of bacteria. These compounds are still active against the Gram-negative bacteria. The antibacterial effects of these enantiomers are less than for the corresponding natural products, showing that there is a preference for the steroconfiguration of the natural products. However, the enantiomers of brevicidine and laterocidine do still provide active and useful alternatives to the natural products.

The enantiomers of brevicidine and laterocidine showed significantly lower activity against S. aureus, a Gram-positive bacteria, than against the Gram-negative bacteria. This shows that enantiomers of brevicidine and laterocidine can selectively target Gram-negative bacteria.

### 3.1.3. In Vitro—Stereochemically Altered Laterocidine Analogues

MIC values were determined for stereochemically altered laterocidine analogues.

It was found that inversion of the stereochemistry at an amino acid residue did not significantly alter the antibiotic activity of the compound.

### 3.1.4. In Vitro—Ser9 and MeDap9 Brevicidine and Laterocidine

MIC values were determined for the Ser9 and MeDap9 analogues of laterocidine and brevicidine against a wide range of bacteria. The results are shown in the table below:

| | Ser9- | MeDap9- | Ser9- | MeDap9- |
|---|---|---|---|---|
| | Brevicidine | Brevicidine | Laterocidine | Laterocidine |
| | (3) | (7) | (4) | (8) |
| E. coli ATCC 25922 | 4 | 16 | 0.5 | 1 |
| E. coli ATCC 25922 MCR-1 | 4 | 8 | 0.5 | 1 |
| E. coli MCR-1 | 2 | 8 | 0.5 | 0.5 |
| E. coli EQAS MCR-2 | 4-8 | 8-16 | 0.5-1 | 0.5 |
| K. pneumoniae ATCC 11228 | 2 | 8 | 1-2 | 1-2 |
| K. pneumoniae ATCC 13883 | 2 | 8 | 1-2 | 1-2 |
| K. pneumonia 2048 | 4 | 8-16 | 2 | 2 |
| K. pneumonia JS-123 | 2 | 4-8 | 0.5 | 0.5 |
| A. baumannii ATCC 17961 | 0.5-1 | 8-16 | 4 | 2 |
| A. baumannii ATCC 17978 | 2 | 16 | 2 | 2 |
| A. baumannii 2018-006 | 2 | 16 | 4 | 4 |
| A. baumannii MDR | 1-2 | 16 | 2 | 4 |
| P. aeruginosa ATCC 27853 | 1 | 2-4 | 0.5-1 | 0.5 |
| P. aeruginosa PAO1 | 2-4 | 4 | 2 | 2 |
| P. aeruginosa NRZ-03961 | 4 | 8 | 1-2 | 2 |
| P. aeruginosa M-120 | 2 | 4 | 1 | 1 |
| S. aureus USA300 | >64 | >64 | >64 | 32 |

Minimum inhibitory concentrations reported in μg/mL.

The results show that the Ser9 and MeDap9 analogues of brevicidine and laterocidine are active against a wide range of bacteria. The Ser9 and MeDap9 analogues of brevicidine and laterocidine are, therefore, useful.

The Ser9 and MeDap9 analogues of brevicidine and laterocidine showed significantly lower activity against S. aureus, a Gram-positive bacteria. This shows that Ser9 and MeDap9 analogues of brevicidine and laterocidine can selectively target Gram-negative bacteria.

### 3.1.5. In Vitro—Laterocidine Lipid Analogues

MIC values were determined for the lipid analogues of laterocidine. The results are shown in the table below:

| | E. | K. | A. | P. |
|---|---|---|---|---|
| Lipid | coli | pneumonia | baumannii | aureuginosa |
| length | ATCC 25922 | ATCC 11228 | ATCC 17978 | PAO1 |
| NH₂ | 32 | >64 | >64 | 64 |
| C₂ | 16 | 16 | 64 | 16 |
| C₄ | 8 | 8 | 4 | 8 |
| C₆ | 1-2 | 2 | 1 | 1-2 |
| C₈ | 0.5-1 | 0.5-1 | 0.5-1 | 1 |
| C₁₀ | 1-2 | 1 | 1 | 1-2 |
| C₁₂ | 4 | 2-4 | 2 | 4 |
| C₁₄ | 8 | 8 | 4 | 8 |
| C₁₆ | 64 | 32 | 4-8 | 64 |

Minimum inhibitory concentrations reported in μg/mL.

The results show that all lipid analogues provided activity against at least the bacteria. Good results were obtained by C4-C14 analogues; excellent results were obtained by C6-C10 analogues and C8-C12 analogues, and the best results were obtained by the C8 analogue.

### 3.1.6. In Vitro—Linear Laterocidine Analogues

Linear laterocidine analogues having the structure:

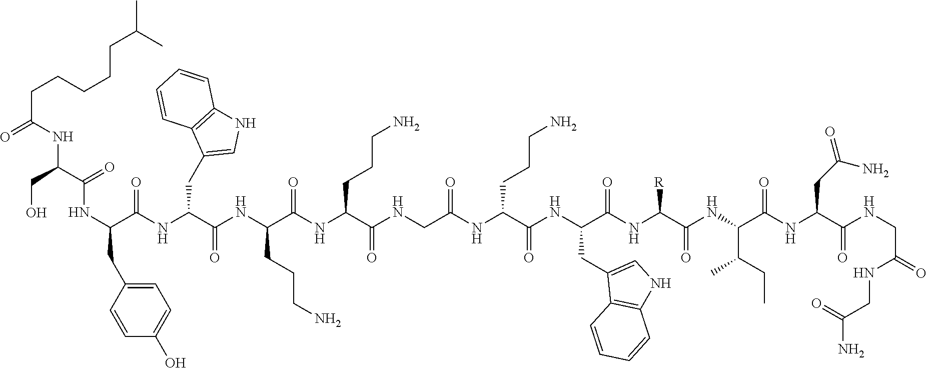

where R represented either:

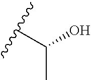 [SMILES: *[C@@H](C)O]

(forming a Thr residue), or

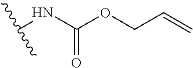 [SMILES: *NC(=O)OCC=C]

(forming a Dap(Alloc) residue), or

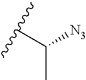 [SMILES: *[C@@H](C)N=[N+]=[N-]]

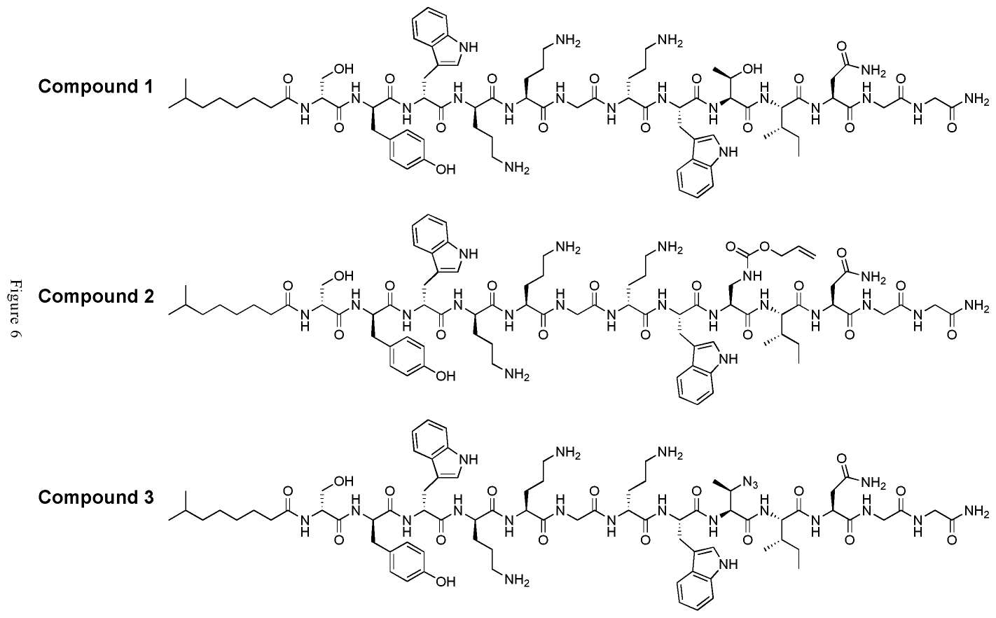

(forming a (2S,3R)-2-amino-3-azidobutanoic acid residue), were tested for inhibition of E. Coli ATCC 25922. FIG. 6 shows the full structure of each compound tested. The results are shown in the table below.

| | E. coli |
|---|---|
| Residue including R | MIC (μg/mL) |
| Thr (linear laterocidine) | 2 |
| Dap(Alloc) | 1-2 |
| (2S,3R)-2-amino-3- | 2-4 |
| azidobutanoic acid | |

Minimum inhibitory concentration of E. coli ATCC 25922 by linear analogues.

These results show that compounds of the present disclosure retain antibacterial activity even when the macrocyclic moiety is opened.

The results in the table above also shows a greater activity against E. coli for analogues having an R-containing residue that is Dap(Alloc), followed by Thr, and then (2S,3R)-2-amino-3-azidobutanoic acid. Whilst the activity of the Thr and (2S,3R)-2-amino-3-azidobutanoic acid-containing analogues is lower than that of the Dap(Alloc) analogue tested in this experiment, the former analogues are still active against the E. coli. Thus, each of the analogues tested in the table above is useful.

### 3.1.7. In Vitro—Alanine-Substituted Linear Laterocidine Analogues

MIC values were determined for the alanine-substituted analogues of laterocidine against E. coli ATCC 25922. The results are shown in the table below:

| Compound | E. coli ATCC 25922 |
|---|---|
| Ala₁ (D-Ser→D-Ala) | 4-8 |
| Ala₂ (D-Tyr→D-Ala) | 8-16 |
| Ala₃ (D-Trp→D-Ala) | 16 |
| Ala₄ (D-Orn→D-Ala) | 32 |
| Ala₅ (Orn→Ala) | 32 |
| Ala₆ (Gly→Ala) | 4-8 |
| Ala₇ (D-Orn→D-Ala) | >64 |
| Ala₈ (Trp→Ala) | >64 |
| Ala₉ (Thr→Ala) | 2 |
| Ala₁₀ (Ile→Ala) | 16 |
| Ala₁₁ (Asn→Ala) | 16 |
| Ala₁₂ (Gly→Ala) | 4 |
| Ala₁₃ (Gly→Ala) | 4 |

^{a}Minimum inhibitory concentrations reported in μg/mL.

It can be seen that there is good tolerance for modification of the structure of linear laterocidine. The replacement of the glycine residues and the serine residue with alanine was particularly well tolerated. Replacement of the tyrosine, tryptophan, isoleucine and asparagine residues with alanine was also well tolerated.

### 3.1.8. In Vitro—Linear Laterocidine Position 9 Analogues

MIC values were determined for the position 9 analogues of linear laterocidine against E. coli ATCC 25922. The results are shown in the table below:

| Compound | E. coli ATCC 25922 |
|---|---|
| Linear Laterocidine (Thr₉-COOH) | 4-8 |
| Thr₉-CONH₂ | 2-4 |
| Dap(Alloc)₉-COOH | 2 |
| Dap(Alloc)₉-CONH₂ | 2 |
| Dap(Alloc)₉-COOAll | 2 |
| Ser₉-CONH₂ | 2 |
| Dap₉-CONH₂ | 4 |
| Leu₉-CONH₂ | 2-4 |
| Phe₉-CONH₂ | 2 |
| Met₉-CONH₂ | 2 |
| Trp₉-CONH₂ | 4 |
| Asn₉-CONH₂ | 4 |
| Gln₉-CONH₂ | 4 |
| Glu(OAll)₉-CONH₂ | 8 |
| MeDap(N3)₉-CONH₂ | 2 |

^{a}Minimum inhibitory concentrations reported in μg/mL.

It can be seen that there is particularly good tolerance for the modification of position 9 of linear laterocidine, especially where the C-terminus of the protein remains amidated.

### 3.1.9. In Vitro—Linear Laterocidine N/C-Termini Analogues

MIC values were determined for the truncated analogues of linear laterocidine against E. coli ATCC 25922. The results are shown in the table below:

| Compound | E. coli ATCC 25922 |
|---|---|
| ΔG13 | 2 |
| ΔG12-G13 | 4 |
| ΔN11- G13 | 4 |
| ΔI10- G13 | 16 |
| ΔT9-G13 | 16 |

^{a}Minimum inhibitory concentrations reported in μg/mL

The results show that the activity of linear laterocidine decreases as the peptide chain is truncated. However, the decrease was small when only one or two amino acids were removed.

### 3.1.10. In Vitro—Other Linear Laterocidine Analogues

MIC values were determined for other analogues of linear laterocidine against E. coli ATCC 25922. The results are shown in the table below:

| Compound | E. coli ATCC 25922 |
|---|---|
| ΔG13-Dap(Alloc)9-isoC9 | 2 |
| ΔG13-Dap(Alloc)9-C8 | 2-4 |
| ΔG13-Ser9-isoC9 | 4 |
| ΔG13-Ser9-C8 | 4 |
| ΔG12-G13-Dap(Alloc)9-isoC9 | 4 |
| ΔG12-G13-Dap(Alloc)9-C8 | 4 |
| ΔG12-G13-Ser9-isoC9 | 4 |
| ΔG12-G13-Ser9-C8 | 4 |

^{a}Minimum inhibitory concentrations reported in μg/mL.

The results show that truncation of the peptide chain (e.g. by one amino acid), modification of the lipid group and modification of amino acid 9 is well tolerated.

### 3.1.11. In Vitro—Relacidine Analogues

MIC values were determined for analogues of relacidine against E. coli ATCC 25922. The results are shown in the table below:

| Compound | E. coli ATCC 25922 |
|---|---|
| R1 | 2 |
| R2 | 2 |
| R3 | 2-4 |
| R4 | 2-4 |
| R5 | 2 |
| R6 | 1 |
| R7 | 1 |
| R8 | 1-2 |
| R9 | 1-2 |
| R10 | 1-2 |
| R11 | 1 |
| R12 | 1-2 |

^{a}Minimum inhibitory concentrations reported in μg/mL.

The results show that modification of the lipid unit as well as amino acids 12 and 13 was well tolerated.

### 3.1.12. Dap9-Relacidamide A

MIC values were determined for relacidamide A against a range of bacteria. The results are shown in the table below:

| Strain | Dap9-Relacidamide A |
|---|---|
| E. coli ATCC 25922 | 2 |
| E. coli ATCC 25922 MCR-1 | 4 |
| E. coli MCR-1 | 2 |
| E. coli EQAS MCR-2 | 2-4 |
| K. pneumoniae ATCC 11228 | 2 |
| K. pneumoniae ATCC 13883 | 2 |
| K. pneumonia 2048 | 2 |
| K. pneumonia JS-123 | 1 |
| A. baumannii ATCC 17961 | 1-2 |
| A. baumannii ATCC 17978 | 4 |
| A. baumannii 2018-006 | 2-4 |
| A. baumannii MDR | 1-2 |
| P. aeruginosa ATCC 27853 | 0.5-1 |
| P. aeruginosa PAO1 | 1-2 |
| P. aeruginosa NRZ-03961 | 2-4 |
| P. aeruginosa M-120 | 1-2 |
| S. aureus USA300 | >64 |

^{a}Minimum inhibitory concentrations reported in μg/mL

The results show that relacidamide A is active against a wide range of bacteria.

Relacidamide A showed significantly lower activity against S. aureus, a Gram-positive bacteria. This shows that relacidamide A can selectively target Gram-negative bacteria.

### 3.1.13. In Vitro—Brevicidine Lipid Analogues

MIC values were determined for the lipid analogues of brevicidine. The results are shown in the table below:

| Analogue | E. coli MIC/μg mL⁻¹ |
|---|---|
| Brev-NH2 | 50 |
| Brev-C2 | 50 |
| Brev-C4 | 25-12.5 |
| Brev-C6 | 25 |
| Brev-C8 | 25 |
| Brev-C10 | 1.56 |
| Brev-C12 | 3.12 |
| Brev-C14 | 50-25 |
| Brev-C16 | >100 |

The results show that all lipid analogues provided activity against at least the bacteria. Reasonable results were obtained by C4-C14 analogues; good results were obtained by C8-C13 analogues, excellent results were obtained by C10-C12 analogues, and the best results were obtained by the C10 analogue.

### 3.1.14. In Vitro—Linear Brevicidine Position 9 Analogues

MIC values were determined for the position 9 analogues of linear brevicidine. The results are shown in the table below:

| Analogue | E. coli MIC/μg mL⁻¹ |
|---|---|
| Und(9-Dap)-NH₂ | 3.1 |
| Und(9-Ser)-NH₂ | 0.78 |
| Und(9-Thr)-NH₂ | 1.56 |
| Und(9-Leu)-NH₂ | 0.78 |
| Und(9-Phe)-NH₂ | <0.20 |
| Und(9-Trp)-NH₂ | 0.78 |
| Und(9-Asp)-NH₂ | 12.5 |
| Und(9-Gln)-NH₂ | 0.78 |
| Und(9-AllGlu)-NH₂ | 0.4 |

The results show that all linear position 9 analogues provided activity against at least the bacteria. Some analogues, such as 9-Phe, 9-AllGlu, 9-Ser, 9-Leu, 9-Trp and 9-Gln provided particularly good results.

### 3.1.15. In Vitro—Linear Brevicidine Analogues

A range of linear analogues having the following general structure:

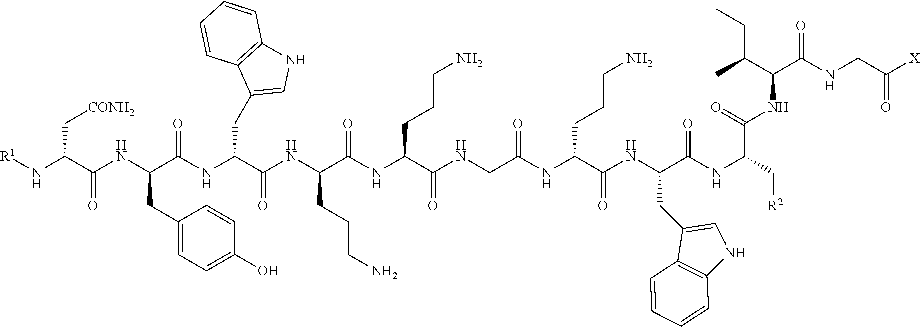

were tested for inhibition of E. Coli NCTC 12241. The results are shown in the table below.

| | AA Residue | | E. coli MIC |
|---|---|---|---|
| R¹ | including R² | X | (μg/mL) |
| MeHex- | Dap(Alloc) | OH | 1.6 |
| Oct- | Dap(Alloc) | OH | 12.5 |
| MeHex- | Ala | OH | 50 |
| Oct- | Ala | OH | 25 |
| MeHex- | Dap(Alloc) | NH₂ | 0.19 |
| Oct | Dap(Alloc) | NH₂ | 0.4 |
| MeHex- | Dap | NH₂ | 3.1 |
| MeHex- | Ser | NH₂ | 0.78 |
| MeHex- | Thr | NH₂ | 1.56 |

Minimum inhibitory concentration (MIC) of E. Coli by linear analogues; “Oct-” represents octanoyl; “MeHex-” represents 4-methylhexanoyl.

It can be seen from the results in the table above that each of the compounds tested was effective against E. coli.

These results show that compounds of the present disclosure retain antibacterial activity even when the macrocyclic moiety is opened.

The results in the table above show that having a C-terminus that is an amide provides greater activity against E. coli than the corresponding carboxylic acid.

Furthermore, results in the table above show that where the amino acid (AA) residue including R² is Dap(Alloc), Dap, Ser or Thr the analogue has greater activity against E. coli compared to where this residue is alanine. Whilst the activity of the analine-containing analogues is lower than that of the other analogues tested in this experiment, the analine-containing analogues are still active against the E. coli. The analine-containing analogues are, therefore, still useful.

The results in the table above also show that there is usually greater activity against E. coli when the R¹ group is MeHex-, as opposed to Oct-.

For comparable compounds Dap(Alloc) group was best, followed by Ser, followed by Thr and then Dap. The results suggest that Ala would be worse than each of the other compounds tested. However, each of the analogues tested was shown to be effective against the E. coli and is, therefore, useful.

### 3.2. Serum Stability Assays

Investigations were performed to determine the stability of brevicidamide and laterocidamide (where X=NH) compared to brevicidine and laterocidine (where X=O) respectively.

10 mg/mL peptide solutions were prepared in in Milli-Q water. Samples were prepared with 42 μL peptide solution and 518 μL human serum and incubated at 37° C. Samples were taken at t=0, 1, 4, 8 and 24 h. To 100 μL of serum, 100 μL of 6% TCA in ACN (containing 0.2 μg/mL D-phenylalanine as internal standard) was added to precipitate the proteins. The samples were vortexed, left for 15 min at room temperature and stored at −20° C. Before analysis the samples were centrifuged for 5 min at 13 000 rpm. The supernatant was analyzed by RP-HPLC using a Shimadzu Prominence-i LC-2030 system with a Dr. Maisch ReproSil Gold 120 C18 column (4.6×250 mm, 5 μm) at 30° C. and equipped with a UV detector monitoring at 220 nm and 254 nm. The following solvent system, at a flow rate of 1 mL/min, was used: solvent A, 0.1% TFA in water/acetonitrile 95/5; solvent B, 0.1% TFA in water/acetonitrile 5/95. Gradient elution was as follows: 100:0 (A/B) for 2 min, 100:0 to 50:50 (A/B) over 45 min, 50:50 (A/B) to 0:100 (A/B) over 1 min, 0:100 (A/B) for 6 min then reversion back to 100:0 (A/B) over 1 min, 100:0 (A/B) for 5 min. The peaks were integrated and normalized to the internal standard.

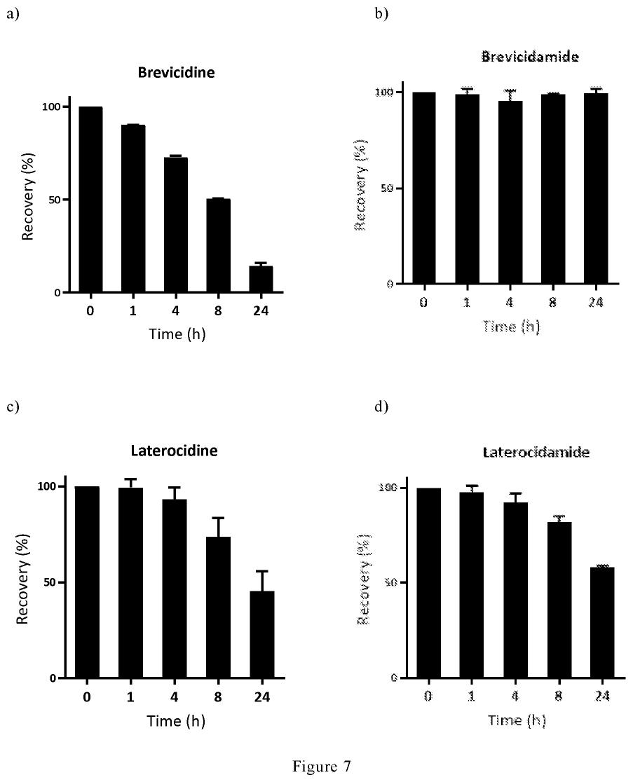

The results of the stability investigations are shown in FIG. 7 of the accompanying drawings. FIG. 7a shows results for brevicidine; FIG. 7b shows results for brevicidamide (Dap9-brevicidine); FIG. 7c shows results for laterocidine; FIG. 7d shows results for laterocidamide (Dap9-laterocidine).

It was found that the ester of brevicidine and laterocidine degrades by hydrolysis. However, replacing the ester for an amide reduces this degradation mechanism in each case, thereby increasing the stability of the analogues. Brevicidamide is particularly stable, showing no significant degradation over the course of the 24-hour study. Laterocidamide was also significantly more stable than laterocidine.

Therefore, analogues with all amides linking the macrocycle (i.e. where X=NH) were found to be more stable than comparative compounds containing an ester in the macrocycle (i.e. where X=O).

### 3.3. Hemolytic Activity

The hemolytic activity of selected analogues against sheep red blood cells was examined. Experiments were performed in triplicate and Triton X-100 used as a positive control. Red blood cells from defibrillated sheep blood obtained from Thermo Fisher were centrifuged (400 g for 15 min at 4° C.) and washed with Phosphate-Buffered Saline (PBS) containing 0.002% Tween20 (buffer) for five times. Then, the red blood cells were normalized to obtain a positive control read-out between 2.5 and 3.0 at 415 nm to stay within the linear range with the maximum sensitivity. A serial dilution of the compounds (200-6.25 μg/mL, 75 μL) was prepared in a 96-well plate. The outer border of the plate was filled with 75 μL buffer. Each plate contained a positive control (0.1% Triton-X final concentration, 75 μL) and a negative control (buffer, 75 μL) in triplicate. The normalized blood cells (75 μL) were added and the plates were incubated at 37° C. for 1 h or 20 h while shaking at 500 rpm. A flat-bottom plate of polystyrene with 100 μL buffer in each well was prepared. After incubation, the plates were centrifuged (800 g for 5 min at room temperature) and 25 μL of the supernatant was transferred to their respective wells in the flat-bottom plate. The values obtained from a read-out at 415 nm were corrected for background (negative control) and transformed to a percentage relative to the positive control.

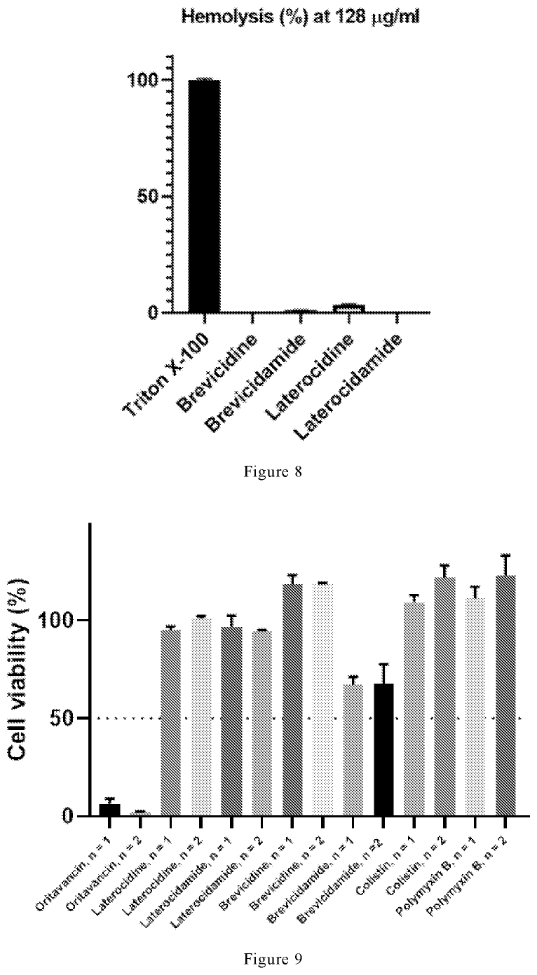

FIG. 8 of the accompanying drawings shows the results of this investigation.

All compounds were found to be non-hemolytic up to the highest concentration tested of 128 μg/mL. No hemolysis was observed for brevicidine or laterocidamide. Very low levels of hemolysis were observed for brevicidamide and laterocidine, far below the levels of the positive control (Triton X-100).

### 3.4. Cytotoxicity

Brevicidine, laterocidine, and their analogues brevicidamide and laterocidamide were tested for cytotoxicity against HepG2 cells using a (3-(4,5-dimethylthiazol-2-yl)-2,5-diphenyltetrazolium bromide (MTT) assay. Oritavancin, colistin and polymyxin B were used as comparative samples. HepG2 cells were seeded at a density of 1.5×10⁴ cells per well in a clear 96-well tissue culture treated plate in a final volume of 100 μL of Dulbecco's Modified Eagle Medium (DMEM), supplemented with Fetal Bovine Serum (1%), Glutamax and Pen/Strep. Cells were incubated for 24 h at 37° C., 7% CO₂ to allow cells to attach to the plates. In addition to a single vehicle control, compounds (diluted from DMSO stock) were added to each well to obtain in a final 128 μg/mL concentration (max. final DMSO concentration 0.5%) for all compounds except for oritavancin which was administered at 50 μM. Incubation was done for 24 h at 37° C., 7% CO₂. After the incubation, MTT was added to each well at a final concentration of 0.40 mg/mL. The plates were then incubated for 2 h at 37° C., 7% CO₂. Medium was carefully removed via suction, and purple formazan crystals were resuspended in 100 μL DMSO. Absorbance was read at 570 nm using a Clariostar plate reader. The data was then analysed with GraphPad Prism software. Technical triplicates for each condition were used, along with biological duplicates.

FIG. 9 of the accompanying drawings shows the results of this investigation.

The cell viability in the presence of the compounds of the present invention was found to be good. All laterocidine- and brevicidine-related compounds showing significantly higher cell viability (i.e. lower cell toxicity) than the licensed medication oritavancin. Brevicidine, laterocidine and laterocidamide were all found to be completely non-toxic under the test conditions.

3.5. In Vivo Tolerability and Efficacy Studies Ethical Issues: Animal experiments were performed under UK Home Office Licenses P89653310 (tolerability and PK) and PA67EOBAA (thigh efficacy), with local ethical committee clearance.

Animal Strain: Mice used in these studies were supplied by Charles River (Margate UK) and were specific pathogen free. The strain of mice used was ICR (also known as CD1 Mice) which is a well characterized outbred murine strain. Mice (male) were 11-15 g on receipt and were allowed to acclimatise for at least 7 days.

Animal Housing: Mice were housed in sterilised individual ventilated cages exposing the mice at all times to HEPA filtered sterile air. Mice had free access to food and water and had aspen chip bedding (changed at least once weekly). The room temperature was 22° C.+/−1° C., with a relative humidity of 60% and maximum background noise of 56 dB. Mice were exposed to 12 h light/dark cycles.

Test compounds: Compound 6 (i.e. laterocidamide) was dissolved in water for injection in which it formed a clear colourless solution. Polymyxin B was dissolved in saline for injection to produce a clear colourless solution.

Tolerability study: The tolerability of compound 6 was assessed in the same mouse strain used for the efficacy studies. Compound 6 was administered via subcutaneous administration route at 3 8-h intervals indicating good tolerability up to 40 mg/kg. The mice used in the tolerability study were naïve and were not immunosuppressed or infected.

Efficacy study: The in vivo efficacy of compound 6 was assessed in a mouse thigh abscess model where both thighs of each mouse were infected with E. coli ATCC 25922 Immunosuppression: Mice were rendered neutropenic with subcutaneous injections of cyclophosphamide at 150 mg/kg 4 days before infection and 100 mg/kg 1 day before infection. The immunosuppression regime leads to neutropenia starting 24 h post administration of the first injection, which continues throughout the study.

Infection: The bacterial strain used was E. coli ATCC 25922. An aliquot of a previously prepared frozen stock of the strain was thawed and diluted in sterile PBS to the desired inoculum just prior to infection. Mice were infected with 0.05 mL of the bacterial strain suspensions by intramuscular (IM) injection under temporary inhaled anaesthesia (2.5% isofluorane for 3-4 min) into both thighs. The inoculum was 6×10⁶ cfu/mL, 3×10⁵ cfu/thigh.

Analgesia: At the time of thigh infection, buprenorphine analgesia was administered at 0.03 mg/kg subcutaneously using a 0.015 mg/mL solution delivered at 2 mL/kg. The same dose was administered again 9 and 17 h post-infection.

Treatment: Compound 6 was administered SC every 8 h starting 1 h post-infection at does of 10, 20, and 40 mg/kg. Additional control groups comprising an infected pre-treatment group, which was euthanised 1 h after infection, a vehicle (WFI) treated group and a group that received comparator Polymyxin B SC every 8 h dosed at 20 mg/kg were included.

Endpoints: 1 h and 20.5 (planned 25) h post-infection, the clinical condition of all animals was assessed prior to humane euthanasia using pentobarbitone overdose, and the thighs were removed and weighed. Thigh samples were homogenized in 3 mL ice cold sterile PBS; the homogenates were quantitatively cultured onto CLED agar and incubated at 37° C. for 18-24 h before colonies were counted.

Data analysis: The data from the culture burdens were analysed using appropriate non-parametric statistical models (Kruskal-Wallis using Conover-Inman to make all pairwise comparisons between groups) with StatsDirect software v. 3.3.5, and compared to vehicle control. For all calculations the thighs from each animal were treated as two separate data points.

It was found that compound 6 (laterocidamide) was well tolerated over the 24 hour period (total daily dose 120 mg/kg).

The efficacy study assessed compound 6 (laterocidamide) for its capacity to reduce thigh infection in neutropenic mice infected with E. coli ATCC 25922. To gain an indication of dose-response, compound 6 was administered subcutaneously q8h at 10, 20, and 40 mg/kg and compared with groups treated with vehicle or polymyxin B as a clinical reference antibiotic administered subcutaneously q8h at 20 mg/kg.

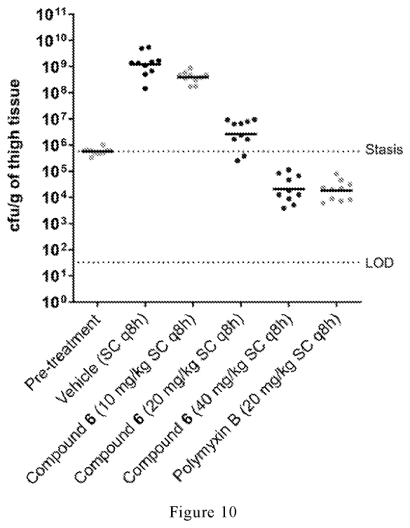

FIG. 10 of the accompanying drawings shows the results of the in vivo efficacy study in a scattergram of mouse thigh burdens (cfu/g) following infection with E. coli ATCC 25922 and treatment with test articles, as indicated on the x-axis. The geometric mean burden of each treatment is indicated by the horizontal bar. LOD=limit of detection.

A clear dose response was observed in the mice treated with compound 6 with the highest 40 mg/kg dose tested resulting in an approximate 5-log reduction in bacterial load relative to the untreated group, an antibacterial effect comparable to that observed for polymyxin B administered at 20 mg/kg. Notably, these in vivo activities reflect the results of the in vitro activity assays wherein compound 6 and polymyxin B were found to have MIC values against E. coli ATCC 25922 of 1.0 and 0.5 g/mL respectively.

### 3.6. Conclusions

The data shows that the compounds of the present invention have good activity against antibiotic resistant strains of bacteria. It has been shown that the compounds retain antibiotic activity even with a good degree of modification.

Detailed studies have been performed into a compound of the present invention (laterocidamide/Compound 6). These studies show that compound 6:
retains a similar activity to commercial “last-resort” antibiotics such as polymyxin B, both in vitro and in vivo;
provides activity against polymyxin B-resistant strains of bacteria;
is stable in serum;
is stable against hemolysis; and
has low cytotoxicity.

The extensive data provided in relation to other compounds of the claimed invention show that it is plausible that one or more of these benefits can be observed for the other compounds of the present invention.

The present disclosure also provides the subject-matter of the following clauses:

1. A compound of formula (I) or formula (II):

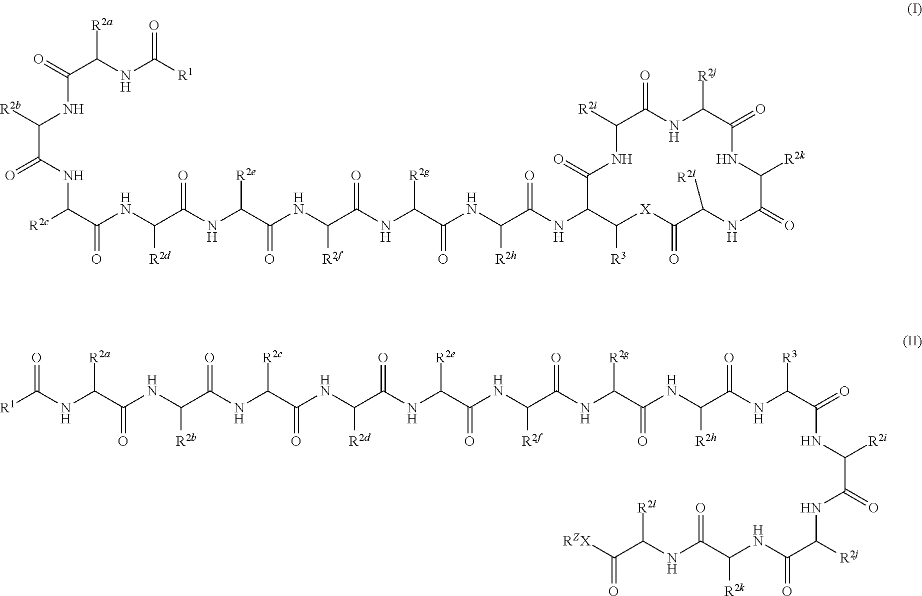

including tautomeric or stereochemically isomeric forms thereof, wherein:
R¹ represents C1-20 alkyl, C2-20 alkenyl, C6-20 aryl, or C4-20 heterocyclyl, each optionally substituted with one or more Y groups,
R^{2a} to R^{2l} each independently represents H, C1-10 alkyl, C2-10 alkenyl, C6-12 aryl, or C4-12 heterocyclyl, each optionally substituted with one or more Y groups,
R³ represents H, C1-10 alkyl, C2-10 alkenyl, C6-12 aryl, or C4-12 heterocyclyl, each optionally substituted with one or more Y groups,
X represents NH, S or O,
each Y independently represents cyano, halogen, N₃, —C(O)R^{Z}, —C(O)OR^{Z}, —OC(O)R^{Z}, —C(O)NHR^{Z}, —NHC(O)R^{Z}, —NHC(O)NHR^{Z}, —NHC(O)OR^{Z}, —OC(O)NHR^{Z}, —OS(O)₂R^{Z}, —S(O)₂NHR^{z}, —NHS(O)₂R^{Z}, —NR^{Z}₂ or —OR^{z}, and
each R^{Z} independently represents H, C1-10 alkyl, C2-10 alkenyl, C6-10 aryl, or C4-10 heterocyclyl;
or an N-oxide thereof or a pharmaceutically acceptable salt thereof or a pharmaceutically acceptable solvate thereof;
   and wherein formula (I) excludes laterocidine:

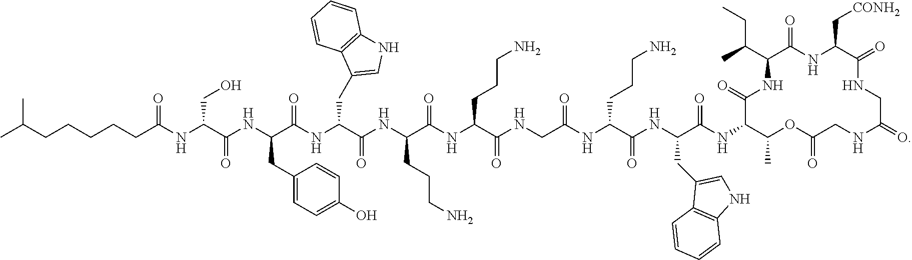 [SMILES: CC[C@H](C)[C@@H]1NC(=O)[C@@H](NC(=O)[C@H](Cc2c[nH]c3ccccc23)NC(=O)[C@@H](CCCN)NC(=O)CNC(=O)[C@H](CCCN)NC(=O)[C@@H](CCCN)NC(=O)[C@@H](Cc2c[nH]c3ccccc23)NC(=O)[C@@H](Cc2ccc(O)cc2)NC(=O)[C@@H](CO)NC(=O)CCCCCC(C)C)[C@@H](C)OC(=O)CNC(=O)CNC(=O)[C@H](CC(N)=O)NC1=O]

2. The compound of clause 1, wherein R¹ represents C6-12 alkyl, C6-12 alkenyl, or C6-12 aryl, each optionally substituted with one or more Y groups.

3. The compound of clause 2, wherein R¹ represents C6-12 alkyl, C6-12 alkenyl, or C6-12 aryl.

4. The compound of any preceding clause, wherein from two to four of the groups R^{2a} to R^{2l} represents C1-10 alkyl, C2-10 alkenyl, C6-12 aryl, or C4-12 heterocyclyl, each substituted with one Y group represented by NH₂.

5. The compound of clause 4, wherein from two to four of the groups R^{2a} to R^{2l} represents C1-10 alkyl or C2-10 alkenyl, each substituted with one Y group represented by NH₂.

6. The compound of any preceding clause, wherein from one to three of the groups R^{2a} to R^{2l} represent a C6-10 heteroaryl group that contains from one to three nitrogen atoms.

7. The compound of any preceding clause, wherein R³ represents H, C1-8 alkyl, C2-8 alkenyl, C6-8 aryl, or C4-8 heterocyclyl, each optionally substituted by one Y group represented by N₃, —C(O)OR^{Z}, C(O)NHR^{Z}, —NHC(O)OR^{Z}, —NR^{Z}₂, or —OR^{Z}.

8. The compound of any preceding clause, wherein X represents NH.

9. A pharmaceutical composition comprising the compound of any one of clauses 1 to 8 and a pharmaceutically acceptable carrier or diluent.

10. A compound according to any one of clauses 1 to 8 or a pharmaceutical composition according to clause 9 for use in therapy.

11. A compound according to any one of clauses 1 to 8 or a pharmaceutical composition according to clause 9 for use as an antibiotic.

12. The compound for use or pharmaceutical composition for use of clause 11, wherein the compound or pharmaceutical composition is for use in treatment or prevention of bacterial infections of Gram-negative bacteria.

13. The compound for use or pharmaceutical composition for use of clause 12, wherein the Gram-negative bacteria is selected from the list consisting of E. coli, K. pneumoniae. A. baumannii, and/or P. aeruginosa.

14. A method of making a pharmaceutical composition according to clause 9, the method comprising the step of mixing a compound according to any one of clauses 1 to 8 with a pharmaceutically acceptable carrier or diluent.

15. A method of treating individuals suffering from bacterial infection, the method comprising administering an effective amount of a compound according to any one of clauses 1 to 8 or a pharmaceutical composition according to clause 9.

16. A compound of formula (III) or (IV):

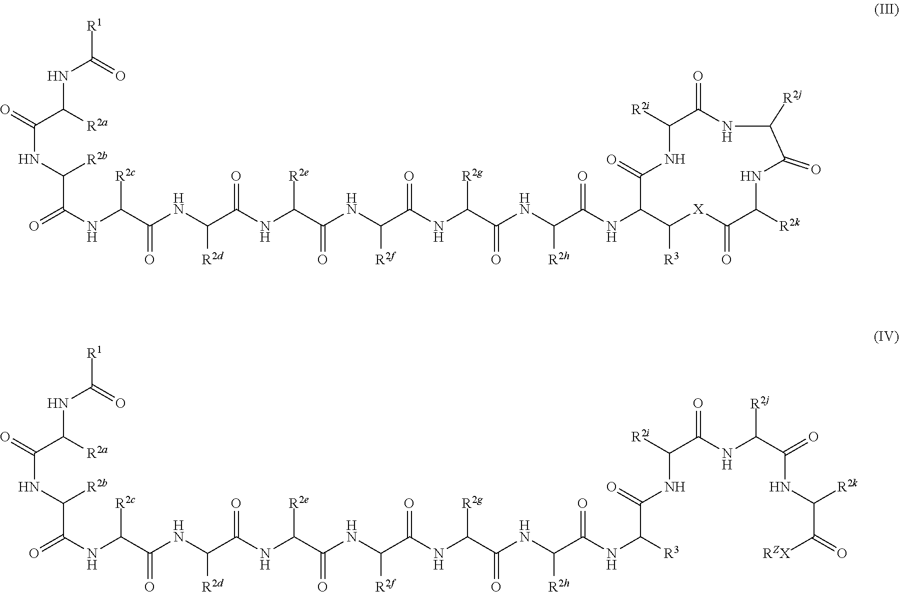

including tautomeric or stereochemically isomeric forms thereof, wherein:
R¹ represents C1-20 alkyl, C2-20 alkenyl, C6-20 aryl, or C4-20 heterocyclyl, each optionally substituted with one or more Y groups,
R^{2a} to R^{2k} each independently represents H, C1-10 alkyl, C2-10 alkenyl, C6-12 aryl, or C4-12 heterocyclyl, each optionally substituted with one or more Y groups,
R³ represents H, C1-10 alkyl, C2-10 alkenyl, C6-12 aryl, or C4-12 heterocyclyl, each optionally substituted with one or more Y groups,
X represents NH, S or O,
each Y independently represents cyano, halogen, —C(O)R^{Z}, —C(O)OR^{Z}, —OC(O)R^{Z}, —C(O)NHR^{Z}, —NHC(O)R^{Z}, —NHC(O)NHR^{Z}, —NHC(O)OR^{Z}, —OC(O)NHR^{Z}, —OS(O)₂R^{Z}, —S(O)₂NHR^{z}, —NHS(O)₂R^{Z}, —NR^{Z}₂ or —OR^{z}, and
each R^{Z} independently represents H, C1-10 alkyl, C2-10 alkenyl, C6-10 aryl, or C4-10 heterocyclyl;
or an N-oxide thereof or a pharmaceutically acceptable salt thereof or a pharmaceutically acceptable solvate thereof;
   and wherein formula (III) excludes brevicidine:

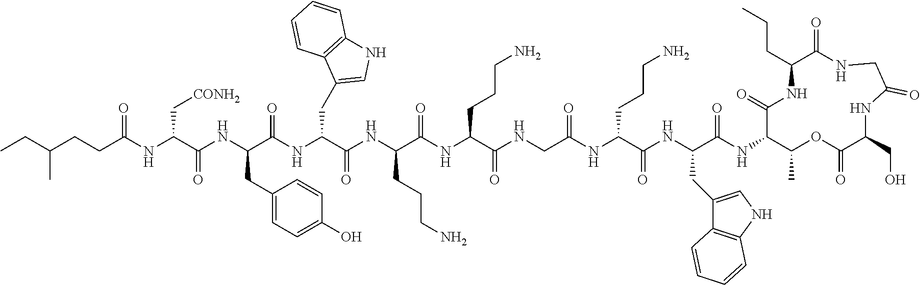 [SMILES: CCC[C@@H]1NC(=O)[C@@H](NC(=O)[C@H](Cc2c[nH]c3ccccc23)NC(=O)[C@@H](CCCN)NC(=O)CNC(=O)[C@H](CCCN)NC(=O)[C@@H](CCCN)NC(=O)[C@@H](Cc2c[nH]c3ccccc23)NC(=O)[C@@H](Cc2ccc(O)cc2)NC(=O)[C@@H](CC(N)=O)NC(=O)CCC(C)CC)[C@@H](C)OC(=O)[C@H](CO)NC(=O)CNC1=O]

17 A compound of formula (V):

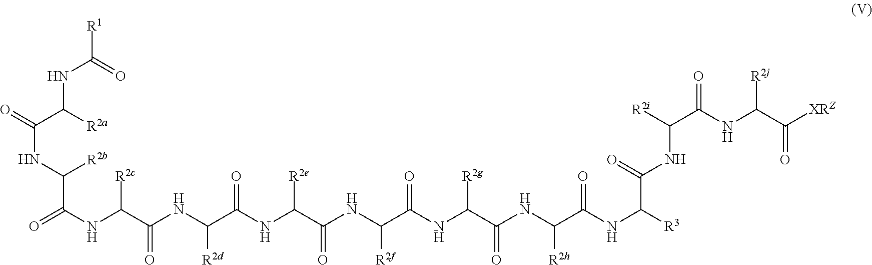

including tautomeric or stereochemically isomeric forms thereof, wherein:
R¹ represents C1-20 alkyl, C2-20 alkenyl, C6-20 aryl, or C4-20 heterocyclyl, each optionally substituted with one or more Y groups,
R^{2a} to R^{2j} each independently represents H, C1-10 alkyl, C2-10 alkenyl, C6-12 aryl, or C4-12 heterocyclyl, each optionally substituted with one or more Y groups,
R³ represents H, C1-10 alkyl, C2-10 alkenyl, C6-12 aryl, or C4-12 heterocyclyl, each optionally substituted with one or more Y groups,
X independently represents NH, S or O,
each Y independently represents cyano, halogen, N₃, —C(O)R^{Z}, —C(O)OR^{Z}, —OC(O)R^{Z}, —C(O)NHR^{Z}, —NHC(O)R^{Z}, —NHC(O)NHR^{Z}, —NHC(O)OR^{Z}, —OC(O)NHR^{Z}, —OS(O)₂R^{Z}, —S(O)₂NHR^{z}, —NHS(O)₂R^{Z}, —NR^{Z}₂ or —OR^{z}, and
each R^{Z} independently represents H, C1-10 alkyl, C2-10 alkenyl, C6-10 aryl, or C4-10 heterocyclyl,
or an N-oxide thereof or a pharmaceutically acceptable salt thereof or a pharmaceutically acceptable solvate thereof.

18. A pharmaceutical composition comprising the compound of clause 16 or clause 17, and a pharmaceutically acceptable carrier or diluent.

19. A compound according to clause 16 or clause 17, or a pharmaceutical composition according to clause 18 for use in therapy.

20. A compound according to the clause 16 or clause 17, or a pharmaceutical composition according to clause 18 for use as an antibiotic.

21. A method of making a pharmaceutical composition according to clause 18, comprising the step of mixing a compound according clause 16 or clause 17 with a pharmaceutically acceptable carrier or diluent.

22. A method of treating individuals suffering from bacterial infection, the method comprising administering an effective amount of a compound clause 16 or clause 17, or a pharmaceutical composition according to clause 18.

1. A compound of formula (I) or formula (II): 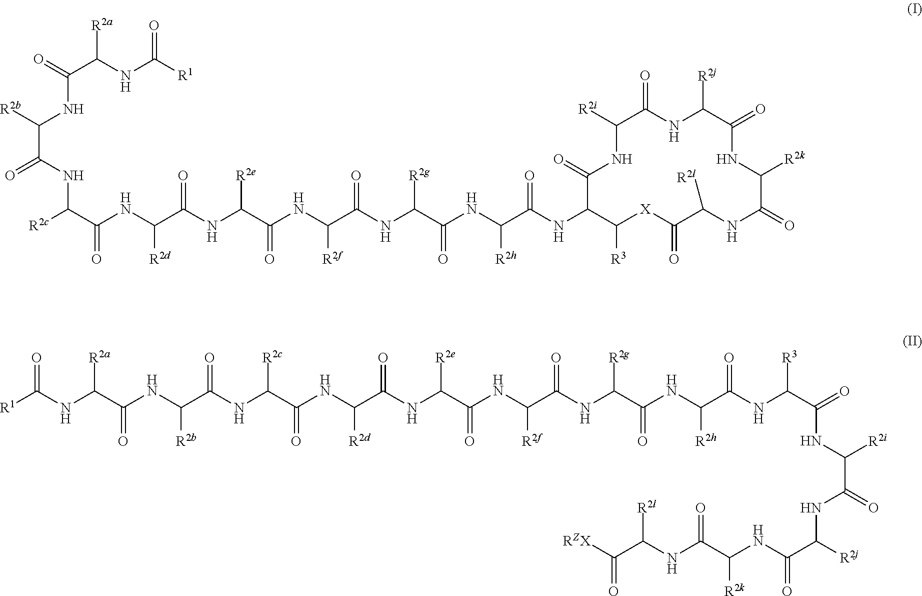
including tautomeric or stereochemically isomeric forms thereof, wherein:
R¹ represents C1-20 alkyl, C2-20 alkenyl, C6-20 aryl, or C4-20 heterocyclyl, each optionally substituted with one or more Y groups,
R^{2a} to R^{2l} each independently represents H, C1-10 alkyl, C2-10 alkenyl, C6-12 aryl, or C4-12 heterocyclyl, each optionally substituted with one or more Y groups,
R³ represents H, C1-10 alkyl, C2-10 alkenyl, C6-12 aryl, or C4-12 heterocyclyl, each optionally substituted with one or more Y groups,
X represents NH, S or O,
each Y independently represents cyano, halogen, N₃, —C(O)R^{Z}, —C(O)OR^{Z}, —OC(O)R^{Z}, —C(O)NHR^{Z}, —NHC(O)R^{Z}, —NHC(O)NHR^{Z}, —NHC(NH)NHR^{Z}, —NHC(O)OR^{Z}, —OC(O)NHR^{Z}, —OS(O)₂R^{Z}, —S(O)₂NHR^{Z}, —NHS(O)₂R^{Z}, —SR^{Z}, —NR^{Z}₂ or —OR^{Z}, and
each R^{Z} independently represents H, C1-10 alkyl, C2-10 alkenyl, C6-10 aryl, or C4-10 heterocyclyl;
or an N-oxide thereof or a pharmaceutically acceptable salt thereof or a pharmaceutically acceptable solvate thereof;
and wherein formula (I) excludes laterocidine: 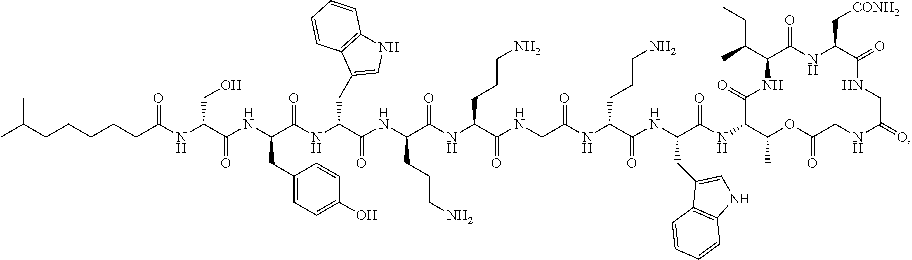 [SMILES: CC[C@H](C)[C@@H]1NC(=O)[C@@H](NC(=O)[C@H](Cc2c[nH]c3ccccc23)NC(=O)[C@@H](CCCN)NC(=O)CNC(=O)[C@H](CCCN)NC(=O)[C@@H](CCCN)NC(=O)[C@@H](Cc2c[nH]c3ccccc23)NC(=O)[C@@H](Cc2ccc(O)cc2)NC(=O)[C@@H](CO)NC(=O)CCCCCC(C)C)[C@@H](C)OC(=O)CNC(=O)CNC(=O)[C@H](CC(N)=O)NC1=O]
wherein formula (I) excludes relacidine A: 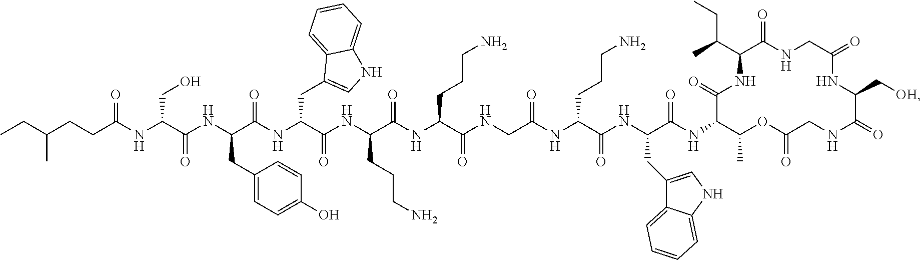 [SMILES: CCC(C)CCC(=O)N[C@H](CO)C(=O)N[C@H](Cc1ccc(O)cc1)C(=O)N[C@H](Cc1c[nH]c2ccccc12)C(=O)N[C@H](CCCN)C(=O)N[C@@H](CCCN)C(=O)NCC(=O)N[C@H](CCCN)C(=O)N[C@@H](Cc1c[nH]c2ccccc12)C(=O)N[C@@H]1C(=O)N[C@@H]([C@@H](C)CC)C(=O)NCC(=O)N[C@@H](CO)C(=O)NCC(=O)O[C@@H]1C]
and wherein formula (I) excludes relacidine B:

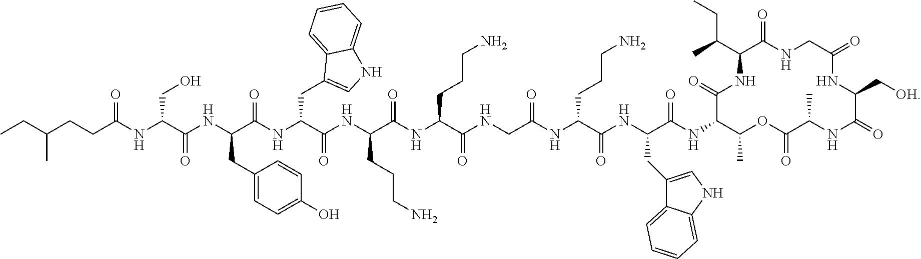 [SMILES: CCC(C)CCC(=O)N[C@H](CO)C(=O)N[C@H](Cc1ccc(O)cc1)C(=O)N[C@H](Cc1c[nH]c2ccccc12)C(=O)N[C@H](CCCN)C(=O)N[C@@H](CCCN)C(=O)NCC(=O)N[C@H](CCCN)C(=O)N[C@@H](Cc1c[nH]c2ccccc12)C(=O)N[C@@H]1C(=O)N[C@@H]([C@@H](C)CC)C(=O)NCC(=O)N[C@@H](CO)C(=O)N[C@@H](C)C(=O)O[C@@H]1C]

2. The compound of claim 1, wherein R¹ represents C6-12 alkyl, C6-12 alkenyl, or C6-12 aryl, each optionally substituted with one or more Y groups.

3. The compound of claim 2, wherein R¹ represents C6-12 alkyl, C6-12 alkenyl, or C6-12 aryl.

4. The compound of claim 1, wherein:
R^{2g} represents C1-10 alkyl, C2-10 alkenyl, C6-12 aryl, or C4-12 heterocyclyl, substituted with from one to three Y groups represented by NR^{Z}₂, and
R^{2h} represents C4-10 alkyl, C4-10 alkenyl, C6-12 aryl, or C4-12 heterocyclyl, optionally substituted with one or more Y groups.

5. The compound of claim 4, wherein R^{2d} and R^{2e} each independently represent C1-10 alkyl, C2-10 alkenyl, C6-12 aryl, or C4-12 heterocyclyl, substituted with from one to three Y groups represented by NR^{Z}₂.

6. The compound of claim 5, wherein:
R^{2b} represents C4-10 alkyl, C4-10 alkenyl, C6-12 aryl, or C4-12 heterocyclyl, each optionally substituted with one or more Y groups,
R^{2c} represents C4-10 alkyl, C4-10 alkenyl, C6-12 aryl, or C4-12 heterocyclyl, optionally substituted with one or more Y groups,
R²ⁱ represents C3, C4 or C5 alkyl or C3, C4 or C5 alkenyl, and
R^{2j} represents C1-8 alkyl, C2-8 alkenyl, C6-8 aryl, or C4-8 heterocyclyl, each substituted with one or more Y group.

7. The compound of claim 1, wherein from two to four of the groups R^{2a} to R^{2l} represents C1-10 alkyl, C2-10 alkenyl, C6-12 aryl, or C4-12 heterocyclyl, each substituted with one Y group represented by NH₂.

8. The compound of claim 7, wherein from two to four of the groups R^{2a} to R^{2l} represents C1-10 alkyl or C2-10 alkenyl, each substituted with one Y group represented by NH₂.

9. The compound of claim 1, wherein from one to three of the groups R^{2a} to R^{2l} represent a C6-10 heteroaryl group that contains from one to three nitrogen atoms.

10. The compound of claim 1, wherein R³ represents H, C1-8 alkyl, C2-8 alkenyl, C6-8 aryl, or C4-8 heterocyclyl, each optionally substituted by one Y group represented by N₃, —C(O)OR^{Z}, C(O)NHR^{Z}, —NHC(NH)NHR^{Z}, —NHC(O)OR^{Z}, —SR^{Z}, —NR^{Z}₂, or —OR^{Z}.

11. The compound of claim 1, wherein X represents NH.

12. A pharmaceutical composition comprising the compound of claim 1 and a pharmaceutically acceptable carrier or diluent.

13-16. (canceled)

17. A method of making a pharmaceutical composition comprising the step of mixing a compound according to claim 1 with a pharmaceutically acceptable carrier or diluent.

18. A method of treating individuals suffering from bacterial infection, the method comprising administering to the individual an effective amount of a compound according to claim 1.

19. The method of claim 18, wherein the compound treats or prevents bacterial infections of Gram-negative bacteria in the individual.

20. The method of claim 19, wherein the Gram-negative bacteria is selected from the list consisting of E. coli, K. pneumoniae, A. baumannii, and/or P. aeruginosa.

21. A method of treating individuals suffering from bacterial infection, the method comprising administering to the individual an effective amount of a pharmaceutical composition according to claim 12.

22. The method of claim 21, wherein the compound treats or prevents bacterial infections of Gram-negative bacteria in the individual.

23. The method of claim 20, wherein the Gram-negative bacteria is selected from the list consisting of E. coli, K. pneumoniae, A. baumannii, and/or P. aeruginosa.


SEQUENCE LISTING (18 sequences)

SEQ ID NO: 1 - protein, 13 aa, Artificial Sequence
  Synthetic Analogue
  MISC_FEATURE (4)..(5): Xaa is Ornithine
  MISC_FEATURE (7)..(7): Xaa is Ornithine
AYWXXGXWTINGG

SEQ ID NO: 2 - protein, 13 aa, Artificial Sequence
  Synthetic Analogue
  MISC_FEATURE (4)..(5): Xaa is Ornithine
  MISC_FEATURE (7)..(7): Xaa is Ornithine
SAWXXGXWTINGG

SEQ ID NO: 3 - protein, 13 aa, Artificial Sequence
  Synthetic Analogue
  MISC_FEATURE (4)..(5): Xaa is Ornithine
  MISC_FEATURE (7)..(7): Xaa is Ornithine
SYAXXGXWTINGG

SEQ ID NO: 4 - protein, 13 aa, Artificial Sequence
  Synthetic Analogue
  MISC_FEATURE (5)..(5): Xaa is Ornithine
  MISC_FEATURE (7)..(7): Xaa is Ornithine
SYWAXGXWTINGG

SEQ ID NO: 5 - protein, 13 aa, Artificial Sequence
  Synthetic Analogue
  MISC_FEATURE (4)..(4): Xaa is Ornithine
  MISC_FEATURE (7)..(7): Xaa is Ornithine
SYWXAGXWTINGG

SEQ ID NO: 6 - protein, 13 aa, Artificial Sequence
  Synthetic Analogue
  MISC_FEATURE (4)..(5): Xaa is Ornithine
  MISC_FEATURE (7)..(7): Xaa is Ornithine
SYWXXAXWTINGG

SEQ ID NO: 7 - protein, 13 aa, Artificial Sequence
  Synthetic Analogue
  MISC_FEATURE (4)..(5): Xaa is Ornithine
SYWXXGAWTINGG

SEQ ID NO: 8 - protein, 13 aa, Artificial Sequence
  Synthetic Analogue
  MISC_FEATURE (4)..(5): Xaa is Ornithine
  MISC_FEATURE (7)..(7): Xaa is Ornithine
SYWXXGXATINGG

SEQ ID NO: 9 - protein, 13 aa, Artificial Sequence
  Synthetic Analogue
  MISC_FEATURE (4)..(5): Xaa is Ornithine
  MISC_FEATURE (7)..(7): Xaa is Ornithine
SYWXXGXWAINGG

SEQ ID NO: 10 - protein, 13 aa, Artificial Sequence
  Synthetic Analogue
  MISC_FEATURE (4)..(5): Xaa is Ornithine
  MISC_FEATURE (7)..(7): Xaa is Ornithine
SYWXXGXWTANGG

SEQ ID NO: 11 - protein, 13 aa, Artificial Sequence
  Synthetic Analogue
  MISC_FEATURE (4)..(5): Xaa is Ornithine
  MISC_FEATURE (7)..(7): Xaa is Ornithine
SYWXXGXWTIAGG

SEQ ID NO: 12 - protein, 13 aa, Artificial Sequence
  Synthetic Analogue
  MISC_FEATURE (4)..(5): Xaa is Ornithine
  MISC_FEATURE (7)..(7): Xaa is Ornithine
SYWXXGXWTINAG

SEQ ID NO: 13 - protein, 13 aa, Artificial Sequence
  Synthetic Analogue
  MISC_FEATURE (4)..(5): Xaa is Ornithine
  MISC_FEATURE (7)..(7): Xaa is Ornithine
SYWXXGXWTINGA

SEQ ID NO: 14 - protein, 12 aa, Artificial Sequence
  Synthetic Analogue
  MISC_FEATURE (4)..(5): Xaa is Ornithine
  MISC_FEATURE (7)..(7): Xaa is Ornithine
SYWXXGXWTING

SEQ ID NO: 15 - protein, 11 aa, Artificial Sequence
  Synthetic Analogue
  MISC_FEATURE (4)..(5): Xaa is Ornithine
  MISC_FEATURE (7)..(7): Xaa is Ornithine
SYWXXGXWTIN

SEQ ID NO: 16 - protein, 10 aa, Artificial Sequence
  Synthetic Analogue
  MISC_FEATURE (4)..(5): Xaa is Ornithine
  MISC_FEATURE (7)..(7): Xaa is Ornithine
SYWXXGXWTI

SEQ ID NO: 17 - protein, 9 aa, Artificial Sequence
  Synthetic Analogue
  MISC_FEATURE (4)..(5): Xaa is Ornithine
  MISC_FEATURE (7)..(7): Xaa is Ornithine
SYWXXGXWT

SEQ ID NO: 18 - protein, 8 aa, Artificial Sequence
  Synthetic Analogue
  MISC_FEATURE (4)..(5): Xaa is Ornithine
  MISC_FEATURE (7)..(7): Xaa is Ornithine
SYWXXGXW